SEQUENCE LISTING (169 sequences)

SEQ ID NO: 1 - protein, 262 aa, Artificial Sequence
  Synthetic sequence human LAT, isoform 1 with leader sequence
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 2 - protein, 3 aa, Artificial Sequence
  Synthetic sequence SH2 domain binding motif
  VARIANT 2: Xaa = any amino acid
YXN

SEQ ID NO: 3 - protein, 4 aa, Artificial Sequence
  Synthetic sequence LAT1 tyrosine-containing fragment sequence
YLVV

SEQ ID NO: 4 - protein, 4 aa, Artificial Sequence
  Synthetic sequence LAT1 tyrosine-containing sequence
YVNV

SEQ ID NO: 5 - protein, 4 aa, Artificial Sequence
  Synthetic sequence LAT1 tyrosine-containing sequence
YENL

SEQ ID NO: 6 - protein, 5 aa, Artificial Sequence
  Synthetic sequence LAT1 tyrosine-containing sequence
SYENE

SEQ ID NO: 7 - protein, 93 aa, Artificial Sequence
  Synthetic sequence Grb2 SH2 domain sequence
WFFGKIPRAKAEEMLSKQRHDGAFLIRESESAPGDFSLSVKFGNDVQHFKVLRDGAGKYFLWVVKFNSLNELVDYHRSTSVSRNQQIFLRDIE

SEQ ID NO: 8 - protein, 60 aa, Artificial Sequence
  Synthetic sequence Variant Grb2 SH2 domain sequence
WFFGKIPRAKAEEMLSKQRHDGAFLIRESESVPGDFALSVKFGNDVQHFLVLRDGAGKYF

SEQ ID NO: 9 - protein, 92 aa, Artificial Sequence
  Synthetic sequence GRAP2 SH2 domain sequence
WFHEGLSRHQAENLLMGKEVGFFIIRASQSSPGDFSISVRHEDDVQHFKVMRDNKGNYFLWTEKFPSLNKLVDYYRTNSISRQKQIFLRDRT

SEQ ID NO: 10 - protein, 98 aa, Artificial Sequence
  Synthetic sequence Src SH2 domain sequence
WYFGKITRRESERLLLNAENPRGTFLVRESETTKGAYCLSVSDFDNAKGLNVKHYKIRKLDSGGFYITSRTQFNSLQQLVAYYSKHADGLCHRLTTVC

SEQ ID NO: 11 - protein, 98 aa, Artificial Sequence
  Synthetic sequence Variant Src SH2 domain sequence
WYFGKITRRESERLLLNAENPRGTFLVRESETTKGAYALSVSDFDNAKGLNVKHYKIRKLDSGGFYITSRTQFNSLQQLVAYYSKHADGLCHRLTTVC

SEQ ID NO: 12 - protein, 98 aa, Artificial Sequence
  Synthetic sequence Fyn SH2 domain sequence
WYFGKLGRKDAERQLLSFGNPRGTFLIRESETTKGAYSLSIRDWDDMKGDHVKHYKIRKLDNGGYYITTRAQFETLQQLVQHYSERAAGLCCRLVVPC

SEQ ID NO: 13 - protein, 98 aa, Artificial Sequence
  Synthetic sequence Variant Fyn SH2 domain sequence
WYFGKLGRKDAERQLLSFGNPRGTFLIRESETTKGAYSLSIRDWDDMKGDHVKHYKIRKLDNGGYYITTRAQFETLQQLVQHYSERAAGLSSRLVVPS

SEQ ID NO: 14 - protein, 98 aa, Artificial Sequence
  Synthetic sequence Fyn SH2 variant consensus sequence
  VARIANT 8: Xaa = R or P
  VARIANT 9: Xaa = K, I, E, Q, T, R, S, D, or L
  VARIANT 11: Xaa = A
  VARIANT 28: Xaa = R
  VARIANT 30: Xaa = S or R
  VARIANT 31: Xaa = E, P, D, V, A, K, G, or Q
  VARIANT 32: Xaa = T, R, S, N, or Y
  VARIANT 33: Xaa = T, F, S, I, V, E, R, P, A, Y, N, or H
  VARIANT 36: Xaa = A, E, D, S, V, G, N, E, or L
  VARIANT 38: Xaa = S, V, A L, or I
  VARIANT 39: Xaa = L
  VARIANT 40: Xaa = S
  VARIANT 53: Xaa = K, V, I, T, or N
  VARIANT 54: Xaa = H or P
  VARIANT 56: Xaa = K, R, L, I, S, or V
  VARIANT 91,92,98: Xaa = any amino acid, preferably each independently C or S
WYFGKLGXXDXERQLLSFGNPRGTFLIXEXXXXKGXYXXXIRDWDDMKGDHVXXYXIRKLDNGGYYITTRAQFETLQQLVQHYSERAAGLXXRLVVPX

SEQ ID NO: 15 - protein, 95 aa, Artificial Sequence
  Synthetic sequence Grap SH2 domain sequence
WYSGRISRQLAEEILMKRNHLGAFLIRESESSPGEFSVSVNYGDQVQHFKVLREASGKYFLWEEKFNSLNELVDFYRTTTIAKKRQIFLRDEEPL

SEQ ID NO: 16 - protein, 97 aa, Artificial Sequence
  Synthetic sequence INPP5D SH2 domain sequence
WNHGNITRSKAEELLSRTGKDGSFLVRASESISRAYALCVLYRNCVYTYRILPNEDDKFTVQASEGVSMRFFTKLDQLIEFYKKENMGLVTHLQYPV

SEQ ID NO: 17 - protein, 100 aa, Artificial Sequence
  Synthetic sequence ITK SH2 domain sequence
WYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPV

SEQ ID NO: 18 - protein, 98 aa, Artificial Sequence
  Synthetic sequence Lck SH2 domain sequence
WFFKNLSRKDAERQLLAPGNTHGSFLIRESESTAGSFSLSVRDFDQNQGEVVKHYKIRNLDNGGFYISPRITFPGLHELVRHYTNASDGLCTRLSRPC

SEQ ID NO: 19 - protein, 109 aa, Artificial Sequence
  Synthetic sequence LCP2 aka SLP-76 SH2 domain sequence
WYVSYITRPEAEAALRKINQDGTFLVRDSSKKTTTNPYVLMVLYKDKVYNIQIRYQKESQVYLLGTGLRGKEDFLSVSDIIDYFRKMPLLLIDGKNRGSRYQCTLTHAA

SEQ ID NO: 20 - protein, 95 aa, Artificial Sequence
  Synthetic sequence Cytoplasmic protein NCK1 SH2 domain sequence
WYYGKVTRHQAEMALNERGHEGDFLIRDSESSPNDFSVSLKAQGKNKHFKVQLKETVYCIGQRKFSTMEELVEHYKKAPIFTSEQGEKLYLVKHL

SEQ ID NO: 21 - protein, 96 aa, Artificial Sequence
  Synthetic sequence Cytoplasmic protein NCK2 SH2 domain sequence
WYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 22 - protein, 96 aa, Artificial Sequence
  Synthetic sequence PIK3R1 SH2 domain 1 sequence
WYWGDISREEVNEKLRDTADGTFLVRDASTKMHGDYTLTLRKGGNNKLIKIFHRDGKYGFSDPLTFSSVVELINHYRNESLAQYNPKLDVKLLYPV

SEQ ID NO: 23 - protein, 95 aa, Artificial Sequence
  Synthetic sequence PIK3R1 SH2 domain 2 sequence
WNVGSSNRNKAENLLRGKRDGTFLVRESSKQGCYACSVVVDGEVKHCVINKTATGYGFAEPYNLYSSLKELVLHYQHTSLVQHNDSLNVTLAYPV

SEQ ID NO: 24 - protein, 96 aa, Artificial Sequence
  Synthetic sequence PIK3R2 SH2 domain 1 sequence
WYWGDISREEVNEKLRDTPDGTFLVRDASSKIQGEYTLTLRKGGNNKLIKVFHRDGHYGFSEPLTFCSVVDLINHYRHESLAQYNAKLDTRLLYPV

SEQ ID NO: 25 - protein, 95 aa, Artificial Sequence
  Synthetic sequence PIK3R2 SH2 domain 2 sequence
WYVGKINRTQAEEMLSGKRDGTFLIRESSQRGCYACSVVVDGDTKHCVIYRTATGFGFAEPYNLYGSLKELVLHYQHASLVQHNDALTVTLAHPV

SEQ ID NO: 26 - protein, 108 aa, Artificial Sequence
  Synthetic sequence PCLG1 SH2 domain 1 sequence
WFHGKLGAGRDGRHIAERLLTEYCIETGAPDGSFLVRESETFVGDYTLSFWRNGKVQHCRIHSRQDAGTPKFFLTDNLVFDSLYDLITHYQQVPLRCNEFEMRLSEPV

SEQ ID NO: 27 - protein, 89 aa, Artificial Sequence
  Synthetic sequence PCLG1 SH2 domain 2 sequence
WYHASLTRAQAEHMLMRVPRDGAFLVRKRNEPNSYAISFRAEGKIKHCRVQQEGQTVMLGNSEFDSLVDLISYYEKHPLYRKMKLRYPI

SEQ ID NO: 28 - protein, 104 aa, Artificial Sequence
  Synthetic sequence PCLG2 SH2 domain 1 sequence
WFHKKVEKRTSAEKLLQEYCMETGGKDGTFLVRESETFPNDYTLSFWRSGRVQHCRIRSTMEGGTLKYYLTDNLTFSSIYALIQHYRETHLRCAEFELRLTDPV

SEQ ID NO: 29 - protein, 90 aa, Artificial Sequence
  Synthetic sequence PCLG2 SH2 domain 2 sequence
WYYDSLSRGEAEDMLMRIPRDGAFLIRKREGSDSYAITFRARGKVKHCRINRDGRHFVLGTSAYFESLVELVSYYEKHSLYRKMRLRYPV

SEQ ID NO: 30 - protein, 97 aa, Artificial Sequence
  Synthetic sequence PTPN6 SH2 domain 1 sequence
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 31 - protein, 104 aa, Artificial Sequence
  Synthetic sequence PTPN6 SH2 domain 2 sequence
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 32 - protein, 99 aa, Artificial Sequence
  Synthetic sequence SH2D1A SH2 domain sequence
VYHGKISRETGEKLLLATGLDGSYLLRDSESVPGVYCLCVLYHGYIYTYRVSQTETGSWSAETAPGVHKRYFRKIKNLISAFQKPDQGIVIPLQYPVEK

SEQ ID NO: 33 - protein, 95 aa, Artificial Sequence
  Synthetic sequence SHB SH2 domain sequence
WYHGAISRGDAENLLRLCKECSYLVRNSQTSKHDYSLSLRSNQGFMHMKLAKTKEKYVLGQNSPPFDSVPEVIHYYTTRKLPIKGAEHLSLLYPV

SEQ ID NO: 34 - protein, 93 aa, Artificial Sequence
  Synthetic sequence Syk SH2 domain 1 sequence
FFFGNITREEAEDYLVQGGMSDGLYLLRQSRNYLGGFALSVAHGRKAHHYTIERELNGTYAIAGGRTHASPADLCHYHSQESDGLVCLLKKPF

SEQ ID NO: 35 - protein, 92 aa, Artificial Sequence
  Synthetic sequence Syk SH2 domain 2 sequence
WFHGKISREESEQIVLIGSKTNGKFLIRARDNNGSYALCLLHEGKVLHYRIDKDKTGKLSIPEGKKFDTLWQLVEHYSYKADGLLRVLTVPC

SEQ ID NO: 36 - protein, 99 aa, Artificial Sequence
  Synthetic sequence TEC SH2 domain sequence
WYCRNMNRSKAEQLLRSEDKEGGFMVRDSSQPGLYTVSLYTKFGGEGSSGFRHYHIKETTTSPKKYYLAEKHAFGSIPEIIEYHKHNAAGLVTRLRYPV

SEQ ID NO: 37 - protein, 95 aa, Artificial Sequence
  Synthetic sequence VAV1 SH2 domain sequence
WYAGPMERAGAESILANRSDGTFLVRQRVKDAAEFAISIKYNVEVKHIKIMTAEGLYRITEKKAFRGLTELVEFYQQNSLKDCFKSLDTTLQFPF

SEQ ID NO: 38 - protein, 97 aa, Artificial Sequence
  Synthetic sequence TXK SH2 domain sequence
WYHRNITRNQAEHLLRQESKEGAFIVRDSRHLGSYTISVFMGARRSTEAAIKHYQIKKNDSGQWYVAERHAFQSIPELIWYHQHNAAGLMTRLRYPV

SEQ ID NO: 39 - protein, 93 aa, Artificial Sequence
  Synthetic sequence ZAP70 SH2 domain 1 sequence
FFYGSISRAEAEEHLKLAGMADGLFLLRQCLRSLGGYVLSLVHDVRFHHFPIERQLNGTYAIAGGKAHCGPAELCEFYSRDPDGLPCNLRKPC

SEQ ID NO: 40 - protein, 92 aa, Artificial Sequence
  Synthetic sequence ZAP70 SH2 domain 2 sequence
WYHSSLTREEAERKLYSGAQTDGKFLLRPRKEQGTYALSLIYGKTVYHYLISQDKAGKYCIPEGTKFDTLWQLVEYLKLKADGLIYCLKEAC

SEQ ID NO: 41 - protein, 97 aa, Artificial Sequence
  Synthetic sequence BLK SH2 domain sequence
WFFRSQGRKEAERQLLAPINKAGSFLIRESETNKGAFSLSVKDVTTQGELIKHYKIRCLDEGGYYISPRITFPSLQALVQHYSKKGDGLCQRLTLPC

SEQ ID NO: 42 - protein, 108 aa, Artificial Sequence
  Synthetic sequence BLNK SH2 domain sequence
WYAGACDRKSAEEALHRSNKDGSFLIRKSSGHDSKQPYTLVVFFNKRVYNIPVRFIEATKQYALGRKKNGEEYFGSVAEIIRNHQHSPLVLIDSQNNTKDSTRLKYAV

SEQ ID NO: 43 - protein, 97 aa, Artificial Sequence
  Synthetic sequence BMX SH2 domain sequence
WFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPV

SEQ ID NO: 44 - protein, 97 aa, Artificial Sequence
  Synthetic sequence BTK SH2 domain sequence
WYSKHMTRSQAEQLLKQEGKEGGFIVRDSSKAGKYTVSVFAKSTGDPQGVIRHYVVCSTPQSQYYLAEKHLFSTIPELINYHQHNSAGLISRLKYPV

SEQ ID NO: 45 - protein, 92 aa, Artificial Sequence
  Synthetic sequence HSH2D SH2 domain sequence
WFHGAISREDAENLLESQPLGSFLIRVSHSHVGYTLSYKAQSSCCHFMVKLLDDGTFMIPGEKVAHTSLDALVTFHQQKPIEPRRELLTQPC

SEQ ID NO: 46 - protein, 98 aa, Artificial Sequence
  Synthetic sequence Lyn SH2 domain sequence
WFFKDITRKDAERQLLAPGNSAGAFLIRESETLKGSFSLSVRDFDPVHGDVIKHYKIRSLDNGGYYISPRITFPCISDMIKHYQKQADGLCRRLEKAC

SEQ ID NO: 47 - protein, 97 aa, Artificial Sequence
  Synthetic sequence PTPN11 SH2 domain 1 sequence
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPL

SEQ ID NO: 48 - protein, 105 aa, Artificial Sequence
  Synthetic sequence PTPN11 SH2 domain 2 sequence
WFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 49 - protein, 99 aa, Artificial Sequence
  Synthetic sequence SH2B2 SH2 domain sequence
WFHGTLSRVKAAQLVLAGGPRNHGLFVIRQSETRPGEYVLTFNFQGKAKHLRLSLNGHGQCHVQHLWFQSVLDMLRHFHTHPIPLESGGSADITLRSYV

SEQ ID NO: 50 - protein, 97 aa, Artificial Sequence
  Synthetic sequence SH2D1B SH2 domain sequence
YYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPI

SEQ ID NO: 51 - protein, 92 aa, Artificial Sequence
  Synthetic sequence SH2D2A SH2 domain sequence
WFHGFITRREAERLLEPKPQGCYLVRFSESAVTFVLTYRSRTCCRHFLLAQLRDGRHVVLGEDSAHARLQDLLLHYTAHPLSPYGETLTEPL

SEQ ID NO: 52 - protein, 100 aa, Artificial Sequence
  Synthetic sequence SH2D3C SH2 domain sequence
WYHGRIPREVSETLVQRNGDFLIRDSLTSLGDYVLTCRWRNQALHFKINKVVVKAGESYTHIQYLFEQESFDHVPALVRYHVGSRKAVSEQSGAIIYCPV

SEQ ID NO: 53 - protein, 94 aa, Artificial Sequence
  Synthetic sequence SH2D4A SH2 domain sequence
WFHGILTLKKANELLLSTGMPGSFLIRVSERIKGYALSYLSEDGCKHFLIDASADAYSFLGVDQLQHATLADLVEYHKEEPITSLGKELLLYPC

SEQ ID NO: 54 - protein, 96 aa, Artificial Sequence
  Synthetic sequence SOCS1 SH2 domain sequence
FYWGPLSVHGAHERLRAEPVGTFLVRDSRQRNCFFALSVKMASGPTSIRVHFQAGRFHLDGSRESFDCLFELLEHYVAAPRRMLGAPLRQRRVRPL

SEQ ID NO: 55 - protein, 98 aa, Artificial Sequence
  Synthetic sequence STAT1 SH2 domain sequence
WNDGCIMGFISKERERALLKDQQPGTFLLRFSESSREGAITFTWVERSQNGGEPDFHAVEPYTKKELSAVTFPDIIRNYKVMAAENIPENPLKYLYPN

SEQ ID NO: 56 - protein, 96 aa, Artificial Sequence
  Synthetic sequence STAT2 SH2 domain sequence
WNDGRIMGFVSRSQERRLLKKTMSGTFLLRFSESSEGGITCSWVEHQDDDKVLIYSVQPYTKEVLQSLPLTEIIRHYQLLTEENIPENPLRFLYPR

SEQ ID NO: 57 - protein, 91 aa, Artificial Sequence
  Synthetic sequence STAT3 SH2 domain sequence
WNEGYIMGFISKERERAILSTKPPGTFLLRFSESSKEGGVTFTWVEKDISGKTQIQSVEPYTKQQLNNMSFAEIIMGYKIMDATNILVSPL

SEQ ID NO: 58 - protein, 96 aa, Artificial Sequence
  Synthetic sequence STAT4 SH2 domain sequence
WIDGYVMGFVSKEKERLLLKDKMPGTFLLRFSESHLGGITFTWVDHSESGEVRFHSVEPYNKGRLSALPFADILRDYKVIMAENIPENPLKYLYPD

SEQ ID NO: 59 - protein, 98 aa, Artificial Sequence
  Synthetic sequence STAT5A SH2 domain sequence
WNDGAILGFVNKQQAHDLLINKPDGTFLLRFSDSEIGGITIAWKFDSPERNLWNLKPFTTRDFSIRSLADRLGDLSYLIYVFPDRPKDEVFSKYYTPV

SEQ ID NO: 60 - protein, 98 aa, Artificial Sequence
  Synthetic sequence STAT5B SH2 domain sequence
WNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPV

SEQ ID NO: 61 - protein, 116 aa, Artificial Sequence
  Synthetic sequence STAT6 SH2 domain sequence
WFDGVLDLTKRCLRSYWSDRLIIGFISKQYVTSLLLNEPDGTFLLRFSDSEIGGITIAHVIRGQDGSPQIENIQPFSAKDLSIRSLGDRIRDLAQLKNLYPKKPKDEAFRSHYKPE

SEQ ID NO: 62 - protein, 98 aa, Artificial Sequence
  Synthetic sequence YES1 SH2 domain sequence
WYFGKMGRKDAERLLLNPGNQRGIFLVRESETTKGAYSLSIRDWDEIRGDNVKHYKIRKLDNGGYYITTRAQFDTLQKLVKHYTEHADGLCHKLTTVC

SEQ ID NO: 63 - protein, 18 aa, Artificial Sequence
  Synthetic sequence linker 218 amino acid sequence
GSTSGSGKPGSGEGSTKG

SEQ ID NO: 64 - protein, 10 aa, Artificial Sequence
  Synthetic sequence linker amino acid sequence
GSGKPGSGEG

SEQ ID NO: 65 - protein, 8 aa, Artificial Sequence
  Synthetic sequence linker amino acid sequence
GKPGSGEG

SEQ ID NO: 66 - protein, 8 aa, Artificial Sequence
  Synthetic sequence linker amino acid sequence
SGKPGSGE

SEQ ID NO: 67 - protein, 6 aa, Artificial Sequence
  Synthetic sequence linker amino acid sequence
  VARIANT (3)...(5): Xaa - Gly or Ser
  VARIANT 6: Xaa = Gly or negitively charged amino acid
BPXXXX

SEQ ID NO: 68 - protein, 110 aa, Artificial Sequence
  Synthetic sequence linker amino acid sequence
  VARIANT (2)...(10): Any one or all of amino acids 2-10 can either be present or absent.
  VARIANT (11)...(110): Any one or all of amino acids 11-110 can either be present or absent.
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 69 - protein, 15 aa, Artificial Sequence
  Synthetic sequence llinker amino acid sequence
GGGGSGGGGSGGGGS

SEQ ID NO: 70 - protein, 18 aa, Artificial Sequence
  Synthetic sequence linker amino acid sequence
GSTSGGGSGGGSGGGGSS

SEQ ID NO: 71 - protein, 12 aa, Artificial Sequence
  Synthetic sequence IgG4 hinge amino acid sequence
  VARIANT 10: Xaa = P or S
ESKYGPPCPXCP

SEQ ID NO: 72 - protein, 28 aa, Artificial Sequence
  Synthetic sequence CD28 transmembrane amino acid sequence
MFWVLVVVGGVLACYSLLVTVAFIIFWV

SEQ ID NO: 73 - protein, 42 aa, Artificial Sequence
  Synthetic sequence 4-1BB intracellular amino acid sequence
KRGRKKLLYIFKQPFMRPVQTTQEEDGCSCRFPEEEEGGCEL

SEQ ID NO: 74 - protein, 112 aa, Artificial Sequence
  Synthetic sequence CD3z intracellular amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 41 aa, Artificial Sequence
  Synthetic sequence CD28 intracellular amino acid sequence
RSKRSRLLHSDYMNMTPRRPGPTRKHYQPYAPPRDFAAYRS

SEQ ID NO: 76 - protein, 41 aa, Artificial Sequence
  Synthetic sequence CD28 intracellular amino acid sequence (LL-GG)
RSKRSRGGHSDYMNMTPRRPGPTRKHYQPYAPPRDFAAYRS

SEQ ID NO: 77 - protein, 42 aa, Artificial Sequence
  Synthetic sequence OX40 intracellular amino acid sequence
ALYLLRRDQRLPPDAHKPPGGGSFRTPIQEEQADAHSTLAKI

SEQ ID NO: 78 - protein, 38 aa, Artificial Sequence
  Synthetic sequence ICOS intracellular amino acid sequence
CWLTKKKYSSSVHDPNGEYMFMRAVNTAKKSRLTDVTL

SEQ ID NO: 79 - protein, 22 aa, Artificial Sequence
  Synthetic sequence Porcine teschovirus-1 2A (P2A) peptide
GSGATNFSLLKQAGDVEENPGP

SEQ ID NO: 80 - protein, 24 aa, Artificial Sequence
  Synthetic sequence Thoseaasigna virus 2A (T2A) peptide
LEGGGEGRGSLLTCGDVEENPGPR

SEQ ID NO: 81 - protein, 20 aa, Artificial Sequence
  Synthetic sequence Equine rhinitis A virus (ERAV) 2A (E2A) peptide
QCTNYALLKLAGDVESNPGP

SEQ ID NO: 82 - protein, 25 aa, Artificial Sequence
  Synthetic sequence Foot-and-Mouth disease virus 2A (F2A) peptide
GSGVKQTLNFDLLKLAGDVESNPGP

SEQ ID NO: 83 - protein, 335 aa, Artificial Sequence
  Synthetic sequence tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 84 - protein, 248 aa, Artificial Sequence
  Synthetic sequence anti-ROR1 scFv
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 85 - protein, 5 aa, Artificial Sequence
  Synthetic sequence R11 ANTIBODY CDRH1
DYPIS

SEQ ID NO: 86 - protein, 16 aa, Artificial Sequence
  Synthetic sequence R11 ANTIBODY CDRH2
FINSGGSTWYASWVKG

SEQ ID NO: 87 - protein, 11 aa, Artificial Sequence
  Synthetic sequence R11 ANTIBODY CDRH3
GYSTYYGDFNI

SEQ ID NO: 88 - protein, 11 aa, Artificial Sequence
  Synthetic sequence R11 ANTIBODY CDRL1
QASQSIDSNLA

SEQ ID NO: 89 - protein, 7 aa, Artificial Sequence
  Synthetic sequence R11 ANTIBODY CDRL2
RASNLAS

SEQ ID NO: 90 - protein, 12 aa, Artificial Sequence
  Synthetic sequence R11 ANTIBODY CDRL3
LGGVGNVSYRTS

SEQ ID NO: 91 - protein, 116 aa, Artificial Sequence
  Synthetic sequence R11 ANTIBODY VH
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISS

SEQ ID NO: 92 - protein, 110 aa, Artificial Sequence
  Synthetic sequence R11 ANTIBODY VL
ELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 93 - protein, 241 aa, Artificial Sequence
  Synthetic sequence R11 SCFV
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 94 - protein, 5 aa, Artificial Sequence
  Synthetic sequence Y31 ANTIBODY CDRH1
SHWMS

SEQ ID NO: 95 - protein, 16 aa, Artificial Sequence
  Synthetic sequence Y31 ANTIBODY CDRH2
IIAASGSTYYANWAKG

SEQ ID NO: 96 - protein, 12 aa, Artificial Sequence
  Synthetic sequence Y31 ANTIBODY CDRH3
DYGDYRLVTFNI

SEQ ID NO: 97 - protein, 11 aa, Artificial Sequence
  Synthetic sequence Y31 ANTIBODY CDRL1
QASQSIGSYLA

SEQ ID NO: 98 - protein, 7 aa, Artificial Sequence
  Synthetic sequence Y31 ANTIBODY CDRL2
YASNLAS

SEQ ID NO: 99 - protein, 9 aa, Artificial Sequence
  Synthetic sequence Y31 ANTIBODY CDRL3
LGSLNSDNV

SEQ ID NO: 100 - protein, 5 aa, Artificial Sequence
  Synthetic sequence 2A2 ANTIBODY CDRH1
DYEMH

SEQ ID NO: 101 - protein, 17 aa, Artificial Sequence
  Synthetic sequence 2A2 ANTIBODY CDRH2
AIDPETGGTAYNQKFKG

SEQ ID NO: 102 - protein, 9 aa, Artificial Sequence
  Synthetic sequence 2A2 ANTIBODY CDRH3
YYDYDSFTY

SEQ ID NO: 103 - protein, 11 aa, Artificial Sequence
  Synthetic sequence 2A2 ANTIBODY CDRL1
KASQNVDAAVA

SEQ ID NO: 104 - protein, 7 aa, Artificial Sequence
  Synthetic sequence 2A2 ANTIBODY CDRL2
SASNRYT

SEQ ID NO: 105 - protein, 9 aa, Artificial Sequence
  Synthetic sequence 2A2 ANTIBODY CDRL3
QQYDIYPYT

SEQ ID NO: 106 - protein, 118 aa, Artificial Sequence
  Synthetic sequence 2A2 ANTIBODY VH
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 107 - protein, 107 aa, Artificial Sequence
  Synthetic sequence 2A2 ANTIBODY VL
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 108 - protein, 237 aa, Artificial Sequence
  Synthetic sequence 2A2 SCFV
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAGGGSGGGSGGGSDIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 109 - protein, 5 aa, Artificial Sequence
  Synthetic sequence R12 ANTIBODY CDRH1
AYYMS

SEQ ID NO: 110 - protein, 17 aa, Artificial Sequence
  Synthetic sequence R12 ANTIBODY CDRH2
TIYPSSGKTYYATWVNG

SEQ ID NO: 111 - protein, 12 aa, Artificial Sequence
  Synthetic sequence R12 ANTIBODY CDRH3
DSYADDGALFNI

SEQ ID NO: 112 - protein, 12 aa, Artificial Sequence
  Synthetic sequence R12 ANTIBODY CDRL1
TLSSAHKTDTID

SEQ ID NO: 113 - protein, 7 aa, Artificial Sequence
  Synthetic sequence R12 ANTIBODY CDRL2
GSYTKRP

SEQ ID NO: 114 - protein, 9 aa, Artificial Sequence
  Synthetic sequence R12 ANTIBODY CDRL3
GADYIGGYV

SEQ ID NO: 115 - protein, 121 aa, Artificial Sequence
  Synthetic sequence R12 ANTIBODY VH
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 116 - protein, 112 aa, Artificial Sequence
  Synthetic sequence R12 ANTIBODY VL
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 117 - protein, 6 aa, Artificial Sequence
  Synthetic sequence ANTI-ROR1 SCFV CDRH1
DTIDWY

SEQ ID NO: 118 - protein, 16 aa, Artificial Sequence
  Synthetic sequence ANTI-ROR1 SCFV CDRH2
VQSDGSYTKRPGVPDR

SEQ ID NO: 119 - protein, 8 aa, Artificial Sequence
  Synthetic sequence ANTI-ROR1 SCFV CDRH3
YIGGYVFG

SEQ ID NO: 120 - protein, 11 aa, Artificial Sequence
  Synthetic sequence ANTI-ROR1 SCFV CDRL1
ASGFDFSAYYM

SEQ ID NO: 121 - protein, 7 aa, Artificial Sequence
  Synthetic sequence ANTI-ROR1 SCFV CDRL2
TIYPSSG

SEQ ID NO: 122 - protein, 9 aa, Artificial Sequence
  Synthetic sequence ANTI-ROR1 SCFV CDRL3
ADRATYFCA

SEQ ID NO: 123 - protein, 5 aa, Artificial Sequence
  Synthetic sequence ANTI-ROR1 SCFV CDRH1
SNLAW

SEQ ID NO: 124 - protein, 16 aa, Artificial Sequence
  Synthetic sequence ANTI-ROR1 SCFV CDRH2
RASNLASGVPSRFSGS

SEQ ID NO: 125 - protein, 8 aa, Artificial Sequence
  Synthetic sequence ANTI-ROR1 SCFV CDRH3
NVSYRTSF

SEQ ID NO: 126 - protein, 11 aa, Artificial Sequence
  Synthetic sequence ANTI-ROR1 SCFV CDRL1
SGSDINDYPIS

SEQ ID NO: 127 - protein, 7 aa, Artificial Sequence
  Synthetic sequence ANTI-ROR1 SCFV CDRL2
INSGGST

SEQ ID NO: 128 - protein, 8 aa, Artificial Sequence
  Synthetic sequence ANTI-ROR1 SCFV CDRL3
YFCARGYS

SEQ ID NO: 129 - protein, 107 aa, Artificial Sequence
  Synthetic sequence ANTI-CD19 FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 130 - protein, 120 aa, Artificial Sequence
  Synthetic sequence ANTI-CD19 FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 131 - protein, 245 aa, Artificial Sequence
  Synthetic sequence ANTI-CD19 FMC63 SCFV
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 132 - DNA, 279 bp, Artificial Sequence
  Synthetic sequence GRB2 SH2 domain
tggttcttcggcaagatccccagagccaaggccgaggaaatgctgagcaagcagagacacgacggcgcctttctgatccgggaatctgaatctgcccctggcgacttcagcctgagcgtgaagttcggcaacgacgtgcagcacttcaaggtgctgagagatggcgccggaaagtacttcctgtgggtcgtgaagtttaacagcctgaacgagctggtggactaccacagatccaccagcgtgtcccggaaccagcagatcttcctgagagacatcgaa

SEQ ID NO: 133 - DNA, 279 bp, Artificial Sequence
  Synthetic sequence GRB2 variant SH2 domain
tggttcttcggcaagatccccagagccaaggccgaggaaatgctgagcaagcagagacacgacggcgcctttctgatccgggaatctgaatctgtccctggcgacttcgccctgagcgtgaagttcggcaacgacgtgcagcacttcctagtgctgagagatggcgccggaaagtacttcctgtgggtcgtgaagtttaacagcctgaacgagctggtggactaccacagatccaccagcgtgtcccggaaccagcagatcttcctgagagacatcgaa

SEQ ID NO: 134 - DNA, 276 bp, Artificial Sequence
  Synthetic sequence GRAP2 SH2 domain
tggtttcacgagggactgagcagacaccaggccgagaatctgctgatgggcaaagaagtgggcttcttcatcatccgggccagccaaagcagccctggcgatttttctatcagcgtgcggcacgaggacgacgtgcagcacttcaaagtgatgcgggacaacaagggcaactacttcctgtggaccgagaagttccccagcctgaacaagctggtggactactaccggaccaacagcatcagccggcagaagcagatcttcctgcgggatagaaca

SEQ ID NO: 135 - DNA, 30 bp, Artificial Sequence
  Synthetic sequence Gly-Ser linker
ggaggtggaggttcaggtggtggaggctct

SEQ ID NO: 136 - DNA, 36 bp, Artificial Sequence
  Synthetic sequence IgG4 hinge
gagagcaagtacggaccgccctgccccccttgccct

SEQ ID NO: 137 - DNA, 84 bp, Artificial Sequence
  Synthetic sequence CD28 TM
atgttctgggtgctggtggtggtcggaggcgtgctggcctgctactcgctgctggtcaccgtggccttcatcatcttttgggtg

SEQ ID NO: 138 - DNA, 129 bp, Artificial Sequence
  Synthetic sequence 4-1BB signaling
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 139 - DNA, 336 bp, Artificial Sequence
  Synthetic sequence CD3z
cgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 140 - DNA, 54 bp, Artificial Sequence
  Synthetic sequence linker 218
ggaagcacatctggctctggcaagcctggatctggcgagggctctaccaaaggc

SEQ ID NO: 141 - DNA, 66 bp, Artificial Sequence
  Synthetic sequence Porcine teschovirus-1 2A (P2A) peptide
ggttccggagccacgaacttctctctgttaaagcaagcaggagacgtggaagaaaaccccggtccc

SEQ ID NO: 142 - DNA, 66 bp, Artificial Sequence
  Synthetic sequence Porcine teschovirus-1 2A (P2A) peptide
ggaagcggagctactaacttcagcctgctgaagcaggctggagacgtggaggagaaccctggacct

SEQ ID NO: 143 - DNA, 63 bp, Artificial Sequence
  Synthetic sequence Thoseaasigna virus 2A (T2A) peptide
ggaagcggagagggcagaggaagtctgctaacatgcggtgacgtcgaggagaatcctggacct

SEQ ID NO: 144 - DNA, 69 bp, Artificial Sequence
  Synthetic sequence Equine rhinitis A virus (ERAV) 2A (E2A) peptide
ggaagcggacagtgtactaattatgctctcttgaaattggctggagatgttgagagcaaccctggacct

SEQ ID NO: 145 - DNA, 75 bp, Artificial Sequence
  Synthetic sequence Foot-and-Mouth disease virus 2A (F2A) peptide
ggaagcggagtgaaacagactttgaattttgaccttctcaagttggcgggagacgtggagtccaaccctggacct

SEQ ID NO: 146 - DNA, 1008 bp, Artificial Sequence
  Synthetic sequence tEGFR
cggaaagtgtgcaacggcatcggcatcggagagttcaaggacagcctgtccatcaacgccaccaacatcaagcactttaagaactgcaccagcatcagcggcgacctgcacattctgcctgtggcctttagaggcgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 147 - DNA, 744 bp, Artificial Sequence
  Synthetic sequence ANTI-ROR1 R12 SCFV
caggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggc

SEQ ID NO: 148 - DNA, 735 bp, Artificial Sequence
  Synthetic sequence ANTI-CD19 FMC63 SCFV
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcctagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatcaggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 149 - DNA, 123 bp, Artificial Sequence
  Synthetic sequence CD28 signaling
cgcagcaagcggagcagaggcggccacagcgactacatgaacatgacccctagacggcctggccccaccagaaagcactaccagccctacgcccctccccgggactttgccgcctacagaagc

SEQ ID NO: 150 - DNA, 1713 bp, Artificial Sequence
  Synthetic sequence A238 R12_GRAP2_SH2 construct
caggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggtttcacgagggactgagcagacaccaggccgagaatctgctgatgggcaaagaagtgggcttcttcatcatccgggccagccaaagcagccctggcgatttttctatcagcgtgcggcacgaggacgacgtgcagcacttcaaagtgatgcgggacaacaagggcaactacttcctgtggaccgagaagttccccagcctgaacaagctggtggactactaccggaccaacagcatcagccggcagaagcagatcttcctgcgggatagaaca

SEQ ID NO: 151 - DNA, 2919 bp, Artificial Sequence
  Synthetic sequence A238 R12_GRAP2_SH2 construct GM-CSF leader EGFRt
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggtttcacgagggactgagcagacaccaggccgagaatctgctgatgggcaaagaagtgggcttcttcatcatccgggccagccaaagcagccctggcgatttttctatcagcgtgcggcacgaggacgacgtgcagcacttcaaagtgatgcgggacaacaagggcaactacttcctgtggaccgagaagttccccagcctgaacaagctggtggactactaccggaccaacagcatcagccggcagaagcagatcttcctgcgggatagaacaggcggcggagaaggcagaggatctctgctgacatgtggcgacgtggaagagaaccccggacctagaatgctgctgctggtcacaagtctgctgctgtgcgagctgccccatcctgcctttctgctgatccccagaaaagtgtgcaacggcatcggcatcggagagttcaaggacagcctgagcatcaatgccaccaacatcaagcactttaagaactgcaccagcatctccggcgacctgcacattctgcctgtggcctttagaggcgacagcttcacccacacacctccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 152 - DNA, 1715 bp, Artificial Sequence
  Synthetic sequence A239 R12_GRB2_SH2 construct
aggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggttcttcggcaagatccccagagccaaggccgaggaaatgctgagcaagcagagacacgacggcgcctttctgatccgggaatctgaatctgcccctggcgacttcagcctgagcgtgaagttcggcaacgacgtgcagcacttcaaggtgctgagagatggcgccggaaagtacttcctgtgggtcgtgaagtttaacagcctgaacgagctggtggactaccacagatccaccagcgtgtcccggaaccagcagatcttcctgagagacatcgaa

SEQ ID NO: 153 - DNA, 2922 bp, Artificial Sequence
  Synthetic sequence A239 R12 GRB2_SH2 construct (GM-CSF leader ′ EGFRt
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggttcttcggcaagatccccagagccaaggccgaggaaatgctgagcaagcagagacacgacggcgcctttctgatccgggaatctgaatctgcccctggcgacttcagcctgagcgtgaagttcggcaacgacgtgcagcacttcaaggtgctgagagatggcgccggaaagtacttcctgtgggtcgtgaagtttaacagcctgaacgagctggtggactaccacagatccaccagcgtgtcccggaaccagcagatcttcctgagagacatcgaaggcggcggagaaggcagaggctctctgcttacatgtggcgacgtggaagagaaccccggacctagaatgctgctgctggtcacatctctgctgctgtgcgagctgccccatcctgcctttctgctgatccctcggaaagtgtgcaacggcatcggcatcggagagttcaaggacagcctgtccatcaacgccaccaacatcaagcactttaagaactgcaccagcatcagcggcgacctgcacattctgcctgtggcctttagaggcgacagcttcacccacacacctccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 154 - DNA, 1659 bp, Artificial Sequence
  Synthetic sequence A240 R12 GRAP2 _SH2 construct
caggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatggtttcacgagggactgagcagacaccaggccgagaatctgctgatgggcaaagaagtgggcttcttcatcatccgggccagccaaagcagccctggcgatttttctatcagcgtgcggcacgaggacgacgtgcagcacttcaaagtgatgcgggacaacaagggcaactacttcctgtggaccgagaagttccccagcctgaacaagctggtggactactaccggaccaacagcatcagccggcagaagcagatcttcctgcgggatagaaca

SEQ ID NO: 155 - DNA, 2865 bp, Artificial Sequence
  Synthetic sequence A240 R12 GRAP2_ SH2 construct (GM-CSF leader ′ EGFRt
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatggtttcacgagggactgagcagacaccaggccgagaatctgctgatgggcaaagaagtgggcttcttcatcatccgggccagccaaagcagccctggcgatttttctatcagcgtgcggcacgaggacgacgtgcagcacttcaaagtgatgcgggacaacaagggcaactacttcctgtggaccgagaagttccccagcctgaacaagctggtggactactaccggaccaacagcatcagccggcagaagcagatcttcctgcgggatagaacaggcggcggagaaggcagaggatctctgctgacatgtggcgacgtggaagagaaccccggacctagaatgctgctgctggtcacaagtctgctgctgtgcgagctgccccatcctgcctttctgctgatccccagaaaagtgtgcaacggcatcggcatcggagagttcaaggacagcctgagcatcaatgccaccaacatcaagcactttaagaactgcaccagcatctccggcgacctgcacattctgcctgtggcctttagaggcgacagcttcacccacacacctccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 156 - DNA, 1662 bp, Artificial Sequence
  Synthetic sequence A241 R12 GRB2__SH2 construct
caggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatggttcttcggcaagatccccagagccaaggccgaggaaatgctgagcaagcagagacacgacggcgcctttctgatccgggaatctgaatctgcccctggcgacttcagcctgagcgtgaagttcggcaacgacgtgcagcacttcaaggtgctgagagatggcgccggaaagtacttcctgtgggtcgtgaagtttaacagcctgaacgagctggtggactaccacagatccaccagcgtgtcccggaaccagcagatcttcctgagagacatcgaa

SEQ ID NO: 157 - DNA, 2868 bp, Artificial Sequence
  Synthetic sequence A241 R12 GRB2__SH2 construct (GM-CSF leader ′ EGFRt
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatggttcttcggcaagatccccagagccaaggccgaggaaatgctgagcaagcagagacacgacggcgcctttctgatccgggaatctgaatctgcccctggcgacttcagcctgagcgtgaagttcggcaacgacgtgcagcacttcaaggtgctgagagatggcgccggaaagtacttcctgtgggtcgtgaagtttaacagcctgaacgagctggtggactaccacagatccaccagcgtgtcccggaaccagcagatcttcctgagagacatcgaaggcggcggagaaggcagaggctctctgcttacatgtggcgacgtggaagagaaccccggacctagaatgctgctgctggtcacatctctgctgctgtgcgagctgccccatcctgcctttctgctgatccctcggaaagtgtgcaacggcatcggcatcggagagttcaaggacagcctgtccatcaacgccaccaacatcaagcactttaagaactgcaccagcatcagcggcgacctgcacattctgcctgtggcctttagaggcgacagcttcacccacacacctccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 158 - DNA, 1650 bp, Artificial Sequence
  Synthetic sequence A281 FMC63 GRB2__SH2 construct
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggttcttcggcaagatccccagagccaaggccgaggaaatgctgagcaagcagagacacgacggcgcctttctgatccgggaatctgaatctgcccctggcgacttcagcctgagcgtgaagttcggcaacgacgtgcagcacttcaaggtgctgagagatggcgccggaaagtacttcctgtgggtcgtgaagtttaacagcctgaacgagctggtggactaccacagatccaccagcgtgtcccggaaccagcagatcttcctgagagacatcgaa

SEQ ID NO: 159 - DNA, 2856 bp, Artificial Sequence
  Synthetic sequence A281 FMC63 GRB2__SH2 construct (GM-CSF leader ′ EGFRt
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggttcttcggcaagatccccagagccaaggccgaggaaatgctgagcaagcagagacacgacggcgcctttctgatccgggaatctgaatctgcccctggcgacttcagcctgagcgtgaagttcggcaacgacgtgcagcacttcaaggtgctgagagatggcgccggaaagtacttcctgtgggtcgtgaagtttaacagcctgaacgagctggtggactaccacagatccaccagcgtgtcccggaaccagcagatcttcctgagagacatcgaaggcggcggagaaggcagaggctctctgcttacatgtggcgacgtggaagagaaccccggacctagaatgctgctgctggtcacatctctgctgctgtgcgagctgccccatcctgcctttctgctgatccctcggaaagtgtgcaacggcatcggcatcggagagttcaaggacagcctgtccatcaacgccaccaacatcaagcactttaagaactgcaccagcatcagcggcgacctgcacattctgcctgtggcctttagaggcgacagcttcacccacacacctccactggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 160 - DNA, 1715 bp, Artificial Sequence
  Synthetic sequence A317 R12 GRB2_super_SH2 construct
aggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctactcgctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggttcttcggcaagatccccagagccaaggccgaggaaatgctgagcaagcagagacacgacggcgcctttctgatccgggaatctgaatctgtccctggcgacttcgccctgagcgtgaagttcggcaacgacgtgcagcacttcctagtgctgagagatggcgccggaaagtacttcctgtgggtcgtgaagtttaacagcctgaacgagctggtggactaccacagatccaccagcgtgtcccggaaccagcagatcttcctgagagacatcgaa

SEQ ID NO: 161 - DNA, 2922 bp, Artificial Sequence
  Synthetic sequence A317 R12 GRB2_super_SH2 construct construct (GM-CSF leader ′ EGFRt
atgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcaattggagccacccgcagttcgaaaaaggaggtggaggttcaggtggtggaggctctgagagcaagtacggaccgccctgccccccttgccctatgttctgggtgctggtggtggtcggaggcgtgctggcctgctactcgctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggaggaaagcccagacggaagaaccctcaagagggcctgtataacgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggaagcacatctggctctggcaagcctggatctggcgagggctctaccaaaggctggttcttcggcaagatccccagagccaaggccgaggaaatgctgagcaagcagagacacgacggcgcctttctgatccgggaatctgaatctgtccctggcgacttcgccctgagcgtgaagttcggcaacgacgtgcagcacttcctagtgctgagagatggcgccggaaagtacttcctgtgggtcgtgaagtttaacagcctgaacgagctggtggactaccacagatccaccagcgtgtcccggaaccagcagatcttcctgagagacatcgaaggcggcggagaaggcagaggctctctgcttacatgtggcgacgtggaagagaaccccggacctagaatgctgctgctggtcacatctctgctgctgtgcgagctgccccatcctgcctttctgctgatccctcggaaagtgtgcaacggcatcggcatcggagagttcaaggacagcctgtccatcaacgccaccaacatcaagcactttaagaactgcaccagcatcagcggcgacctgcacattctgcctgtggcctttagaggcgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 162 - protein, 4 aa, Artificial Sequence
  Synthetic sequence ITAM motif
  VARIANT (2)...(3): Xaa = any amino acid
  VARIANT 4: Xaa = L or I
YXXX

SEQ ID NO: 163 - protein, 16 aa, Artificial Sequence
  Synthetic sequence ITAM motif
  VARIANT (2)...(3): Xaa = any amino acid
  VARIANT 4: Xaa = L or I
  VARIANT (5)...(12): Xaa = any amino acid
  VARIANT (11)...(12): Any one or all of amino acids 11-12 may be present or absent.
  VARIANT (14)...(15): Xaa = any amino acid
  VARIANT (16)...(16): Xaa = L or I
YXXXXXXXXXXXYXXX

SEQ ID NO: 164 - DNA, 544 bp, Artificial Sequence
  Synthetic sequence EF1 promoter
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 165 - DNA, 66 bp, Artificial Sequence
  Synthetic sequence exemplary GM-CSF leader coding sequence
atgctgctgctggtgacaagcctgctgctgtgcgagctgccccaccccgcctttctgctgatcccc

SEQ ID NO: 166 - protein, 8 aa, Artificial Sequence
  Synthetic sequence EBV RAK peptide
RAKFKQLL

SEQ ID NO: 167 - protein, 9 aa, Artificial Sequence
  Synthetic sequence Meganuclease
LAGLIDADG

SEQ ID NO: 168 - protein, 6 aa, Artificial Sequence
  Synthetic sequence Meganuclease
GIYYIG

SEQ ID NO: 169 - protein, 5 aa, Artificial Sequence
  Synthetic sequence Meganuclease
  VARIANT 3: Xaa = D or E
  VARIANT 4: Xaa = any amino acid
PDXXK

(19) United States
(12) Patent Application Publication
(10) Pub. No.: US 2022/0401537 A1
(43) Pub. Date: Dec. 22, 2022

(54) CHIMERIC RECEPTOR PROTEINS AND USES THEREOF

(71) Applicant: FRED HUTCHINSON CANCER RESEARCH CENTER, Seattle, WA (US)

(72) Inventors: Alexander SALTER, Seattle, WA (US); Stanley R. RIDDELL, Sammamish, WA (US); Anusha RAJAN, Woodinville, WA (US)

(21) Appl. No.: 17/760,747

(22) PCT Filed: Sep. 15, 2020
(86) PCT No.: PCT/US2020/050916

§ 371 Date: Mar. 15, 2022

Related U.S. Application Data
(60) Provisional application No. 62/901,186, filed on Sep. 16, 2019.

(51) Int. Cl. A61K 39/00; C07K 14/71; C07K 14/725; C07K 14/705; C07K 14/73; C07K 16/28; A61P 35/00; C12N 15/62; C12N 15/86; C12N 5/0783

(52) U.S. Cl. CPC A61K 39/001112; C07K 14/71; C07K 14/7051; C07K 14/70596; C07K 14/705; C07K 14/70521; C07K 14/70517; C07K 14/70514; C07K 14/70578; C07K 16/2803; A61K 39/001102; A61P 35/00; C12N 15/625; C12N 15/86; C12N 5/0636; C07K 2319/00; C07K 2317/622; C07K 2317/569; A61K 39/00; A61K 38/00

(57) ABSTRACT

The present disclosure provides fusion proteins with improved signaling properties. Disclosed embodiments include fusion proteins that comprise an extracellular component comprising a target-binding domain, a transmembrane domain, and an intracellular component comprising a SH2 domain or a functional portion or variant thereof, and have improved signaling in response to antigen-binding, including of solid-tumor antigens with low levels of expression. Recombinant host cells expressing the fusion proteins, and polynucleotides encoding the fusion proteins, are also provided, as are compositions and methods comprising the same.

### STATEMENT OF GOVERNMENT INTEREST

This invention was made with government support under CA136551 awarded by the National Institutes of Health. The government has certain rights in the invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_474WO_SEQUENCE_LISTING.txt. The text file is 134 KB, was created on Sep. 15, 2020, and is being submitted electronically via EFS-Web.

### BACKGROUND

Adoptive transfer of genetically modified T cells has emerged as a potent therapy for various malignancies. The most widely employed strategy has been infusion of patient-derived T cells expressing chimeric antigen receptors (CARs) targeting tumor associated antigens. This approach has numerous theoretical advantages, including the ability to target T cells to any cell surface antigen, circumvent loss of major histocompatibility complex as a tumor escape mechanism, and employ a single vector construct to treat any patient, regardless of human leukocyte antigen haplotype. For example, CAR clinical trials for B-cell non-Hodgkin's lymphoma (NHL) have targeted CD19, CD20, or CD22 antigens that are expressed on malignant lymphoid cells as well as on normal B cells (Brentjens et al., Sci Transl Med 2013; 5(177):177ra38; Haso et al., Blood 2013; 121(7):1165-74; James et al., J Immunol 2008; 180(10):7028-38; Kalos et al., Sci Transl Med 2011; 3(95):95ra73; Kochenderfer et al., J Clin Oncol 2015; 33(6):540-9; Lee et al., Lancet 2015; 385(9967):517-28; Porter et al., Sci Transl 25 Med 2015; 7(303):303ra139; Savoldo et al., J Clin Invest 2011; 121(5):1822-6; Till et al., Blood 2008; 112(6):2261-71; Till et al., Blood 2012; 119(17):3940-50; Coiffier et al., N Engl J Med 2002; 346(4):235-42).

However, adoptive cell therapies are still developing, and new strategies are needed, for example, to improve cellular immunotherapy when targeting antigens in vivo, including in the context of solid tumors. The presently disclosed embodiments address these needs and provide other related advantages.

### BRIEF DESCRIPTION OF THE DRAWINGS

FIGS. 1A-1H show design and testing of bi-specific T cells for selective analysis of TCR- and CAR-induced signaling and effector functions in a single-cell-type population. (A) Schematic of bi-specific T cells that express an endogenous EBV-specific T cell receptor (TCR) and were transduced to express a ROR1-specific chimeric antigen receptor (CAR). (B) Flow cytometry analysis of EBV-tetramer binding and CD19t expression in expanded T cells. FACS plot shows stained (black) and isotype (grey) CD8⁺ singlet lymphocytes. (C) Kinetics of Ca2⁺ mobilization in populations of cells stimulated by ROR1- or SCT-containing bilayers was measured by Fluo-4 AM intensity of individual cell responses after exposure to bilayers. Traces represent the fraction of cells above an activation threshold at any given time. Antigen density was modulated by the mole fraction of biotinylated lipids in the supported lipid bilayer—0.005% or 0.01%. (D) Schematic of magnetic beads coated with HLA-B8/EBV-RAK single chain trimer (SCT) or ROR1 ectodomain. (E) Western blot analysis for CD3ζ, CD3ζ pTyr¹⁴², PLC-γ1, and PLC-γ1 pTyr⁷⁸³ in lysates from T cells incubated for 45 minutes with various amounts of microbeads coated with SCT or ROR1. (F) Schematic of magnetic beads coated with SCT and CD28 mAb and Western blot analysis for CD3ζ, CD3ζ pTyr¹⁴², and PLC-γ1 pTyr⁷⁸³ in lysates from T cells incubated for 45 minutes with uncoated (0), SCT, or SCT/CD28 microbeads. 7.5 μL beads were used per million cells. (G) Flow cytometry analysis of T cell proliferation as measured by CFSE dye dilution at 72 hours after microbead stimulation. Histogram plot of CD8⁺ lymphocytes treated with uncoated, SCT, or SCT/CD28 microbeads. (H) Western blot analysis for CD3ζ, CD3ζ pTyr¹⁴², SLP-76, SLP-76 pSer³⁷⁶, PLC-γ1, and PLC-γ1 pTyr⁷⁸³ in T cell lysates after stimulation with beads or cells for 45 minutes. Data are representative of 5 (B) 3 (C) or 2 (H) independent experiments.

FIGS. 2A-2I show identification of protein phosphorylation events by mass spectrometry (MS) following TCR or CAR stimulation. (A) Schematic of experimental design stimulating bi-specific T cells with microbeads coated with TCR or CAR antigens. (B) Flow cytometry analysis of CD45RO, CD62L, CD28 and DNA content in expanded T cells prior to MS signaling analysis. Histograms show stained (black) and isotype control (grey) CD8⁺ singlet lymphocytes. DNA content was measured by staining with propidium iodide after fixation and permeabilization. Frequencies of cells in G0/G1, S, and G2 phases (from left to right) are indicated by the gates. (C) X-Y plot shows mean IFN-γ concentration in supernatant 24 hours after incubation of bi-specific T cells from two donors with EBV-RAK peptide pulsed K562/HLA-B8 cells. Mean was calculated from technical duplicates. (D) Venn diagram of the overlap among PO₄ sites from 3 tandem MS/MS experiments. (E) Histogram of the standard deviation (SD) of log 2FC values across the 3 tandem MS/MS experiments. (F)-(I) Fold change of the indicated PO₄ sites identified by tandem MS/MS. Data are means from 2 or 3 experiments.

FIGS. 3A-3I show that CAR stimulation promotes less intense phosphorylation of CD3 chains and proximal TCR signaling adaptors as compared to TCR stimulation. (A) Comparison of the log 2FC of PO₄ sites identified by tandem MS/MS 10 minutes after TCR or CAR stimulation. Green and red dots specify sites that possessed mean log 2FC values differing by >1 between TCR- and CAR-stimulated samples. (B) Fold change of the indicated PO₄ sites identified by tandem MS/MS. Data are means from 2 or 3 experiments. (C) Heat map shows mean log 2FC values of select genes identified in (A) at the 10-minute time point. Data are means from 2 or 3 experiments. (D)-(E) Comparison of the log 2FC of PO₄ sites identified by tandem MS/MS at the indicated timepoints after TCR or CAR stimulation. Dots above and below dashed lines specify sites that possessed mean log 2FC values differing by >1 between TCR- and CAR-stimulated samples. (F) Fold change of the indicated PO₄ sites identified by tandem MS/MS. Data are means from 2 or 3 experiments. (G) Western blot analysis for LAT and LAT pTyr¹⁹¹ in bi-specific T cell lysates after 10 minutes of stimulation. Blots are representative of 2 independent experiments. (H) Western blot analysis for LAT, LAT pTyr¹⁹¹, SLP-76, SLP-76 pSer³⁷⁶, PLC-γ1, and PLC-γ1 pTyr⁷⁸³ in lysates from bi-specific T cells expressing a 4-1BB/CD3ζ CAR after 10 minutes of stimulation. (I) Fold change of normalized band intensity are means±SEM from three unique T cell donors relates to representative Western Blot data shown in (H). The indicated P values were calculated by repeated-measures one-way ANOVA with Tukey's multiple comparisons test comparing samples at equivalent time points.

FIGS. 4A-4L show design and testing of exemplary fusion protein designs according to the present disclosure. (A) Schematic of LAT_{TMD} CAR designs. (B) Flow cytometry analysis of EGFRt and STII (LAT_{TMD} CAR) expression in FACS-purified and expanded CD8⁺EGFRt⁺ T cells. (C) Western blot analysis for CD3ζ in T cell lysates as in (B). (D) Concentration of the indicated cytokines in supernatant 24 hours after co-culture of CAR T cells with K562/ROR1 tumor cells. (E) Schematic and representative fluorescence microscopy images of various LAT_{TMD}-eGFP fusion proteins. Images are representative of n=2 healthy blood donors with at least 10 cells visualized per experimental condition. (F) Schematic of CAR designs that included a ROR1-specific scFv derived from R12 antibody and a GRB2- or GRAP2-derived SH2 domain, with (bottom) or without (middle) a linker separating the SH2 domain from the CD3ζ domain. A reference 4-1BB/CD3ζ CAR construct without an SH2 is illustrated schematically at top. (G) Western blot analysis for CD3ζ in CAR T cell lysates. (H) Flow cytometric analysis of EGFRt and STII (CAR) expression on sort purified and expanded CD8⁺EGFRt⁺ T cells. (I) Mean±SD of fold change in median fluorescence intensity (MFI) of 4-1BB/CD3ζ/link GRB2 CAR T cells relative to 4-1BB/CD3ζ CAR T cells. N=5 healthy T cell donors. A one-sample t test with H₀=1 was used to assess significance. (J-K) Mean±SEM fold change of cytokine concentration in cellular supernatant 24 hours after co-culture of CAR T cells with K562/ROR1 (J) or MDA-MB-231 (K) tumor cells. n=4 healthy T cell donors. (L) Mean±SEM cytokine concentration in cellular supernatant 24 hours after co-culture of CAR T cells with K562 tumor cells. n=4 healthy T cell donors.

FIGS. 5A-5G show that fusion proteins according to the present disclosure possess improve antigen sensitivity. (A-C) Fluo-4 Ca²⁺ mobilization measurements for cells stimulated on ROR1-labeled bilayers. (A, B) Traces represent the fraction of cells with high levels of intracellular Ca²⁺ across time after exposure to bilayers. Data from 4 independent experiments are accumulated for each trace, and fill represents the SD between experiments. (C) Fraction of cells responding at 20 min after exposure to bilayer for a range of ROR1 densities, determined by the mol % of biotinylated lipids in the bilayer. Each data point represents 3-4 independent experiments. Error bars are SEM. (D) Western blot analysis for LAT pTyr¹⁹¹, SLP-76 pSer³⁷⁶, PLC-γ1 pTyr⁷⁸³, CD3ζ, and CD3ζ pTyr¹⁴² in T cell lysates after 10 of incubation with ROR1 beads or mock beads. Blots are representative of three independent experiments using T cells prepared from three unique donors. (E) Fold change of normalized Western blot band intensity are means±SEM from three unique T cell donors as in (D). The indicated P values were calculated by repeated-measures one-way ANOVA with Tukey's multiple comparisons test comparing samples at equivalent time points. (F-G) Graphs show mean±SEM of the percentage of T cells staining positive for CD25 and CD69 (F) or IFN-γ concentration in supernatant (G) after twenty-four hours of stimulation with the indicated amounts of ROR1 coated onto plates. N=three independent experiments utilizing T cells from three unique donors.

FIGS. 6A-6I show that fusion proteins according to the present disclosure improve antitumor function in vivo. (A) Measurement of ROR1 expression on various tumor cell lines by flow cytometry. Histograms show stained (black) and isotype control (grey) singlets. (B) Survival of NSG mice engrafted with ROR1^{int} Jeko-1 cells and treated with CD28/CD3ζ, 4-1BB/CD3ζ or 4-1BB/CD3ζ/link_GRB2 CAR T cells. (C-E) Mean±SEM radiance of luciferase-expressing ROR1^{low} MDA-MB-231 tumors over time (C), at day 27 (D), and at day 34 (E) post-tumor inoculation. N=5 mice per group. Group means were compared using a one-way ANOVA with Tukey's post-test. (F) Mean±SEM of CAR T cell frequency in tumors at day 20. N=7 mice per group. (G) Survival of NSG mice engrafted with CD19high Raji cells and treated with CD28/CD3ζ, 4-1BB/CD3ζ or 4-1BB/CD3ζ/link_GRB2 anti-CD19 CAR T cells. (H) Mean±SEM of PD-1 (left) and LAG3 (right) median fluorescence intensity (MFI) of CAR T cells in bone marrow at day 20. N=7 mice per group. (I) Mean±SEM of CAR T cell frequency in bone marrow at day 20. N=7 mice per group.

FIGS. 7A and 7B relate to primary T cells transduced to express an anti-ROR1 chimeric antigen receptor containing a variant GRB2 SH2 domain (“Superbinder”). (A) Flow cytometric analysis of EGFRt and STII (CAR) expression on sort purified and expanded CD8⁺EGFRt⁺ T cells. (B) Proliferation of primary CD8⁺ T cells expressing the indicated constructs when co-cultured with K562/ROR1 target cells. In (A and B), untransduced CD8⁺ T cells are shown as a control.

### DETAILED DESCRIPTION

The present disclosure provides chimeric receptor proteins with improved signaling properties over existing immunoreceptor proteins (e.g., chimeric antigen receptors (CARs), T cell receptors (TCRs), or the like), which improved properties can include, in certain embodiments, initiating, generating, propagating, and/or amplifying a signal or other activity in a host cell expressing the fusion protein when the fusion protein binds to an antigen or other target. Improved properties are present even when the target is expressed at a low level or an intermediate level (e.g., as compared to a reference level, such as a typical or standard expression level of the target for a disease condition in a cell, tissue, or subject) by a target-expressing cell.

Exemplary fusion proteins of this disclosure comprise (a) an extracellular component comprising a binding domain that specifically binds to a target, such as an antigen; (b) a transmembrane domain; and (c) an intracellular component comprising a SH2 domain or a functional portion or variant thereof. In certain embodiments, the SH2 domain or functional portion or variant thereof is from Grb2, Grap2, Fyn, Src, Grap, CRLK, INPP5D, ITK, LCK, SLP-76, NKC1, NCK2, PIK3R1, PIK3R2, PLCG1, PLCG2, PTPN6, SH2D1A, SHB, Syk, TEC, VAV1, TXK, ZAP70, BLK, BLNK, BMX, BTK, HSH2D, LYN, PTPN11, SH2B2, SH2D1B, SH2D2A, SH2D3C, SH2D4A, SOCS1, STAT1, STAT2, STAT3, STAT4, STAT5A, STAT5B, STAT6, or YES1.

In certain embodiments, the intracellular component of the fusion protein further comprises an effector domain or functional portion or variant thereof, a costimulatory domain or functional portion or variant thereof, or both.

Disclosed fusion proteins also maintain one or more functions when the target is expressed at a high level. In other words, improved sensitivity to a target is not accompanied by loss of function, or by substantial loss of function, when the target is highly expressed. For cells expressing a disclosed fusion protein, enhanced target sensitivity of a fusion protein does not result in upregulation or substantial upregulation of an exhaustion-associated inhibitor molecule, such as PD-1 and/or LAG-3, as compared to a reference cell expressing a reference protein that does not include a SH2 domain, as provided herein. Further, disclosed fusion proteins provide enhanced intracellular signaling and target sensitivity without undesirably increasing cellular production of pro-inflammatory cytokines, and may present a reduced risk of cytokine-associated toxicities. Cells expressing disclosed a fusion protein can also localize to, expand at, and/or persist at a target-expressing site for longer than cells expressing a reference or comparator fusion protein that does not comprise the SH2 domain or functional portion or variant thereof, as provided herein.

The presently disclosed fusion proteins can be useful in cellular immunotherapies comprising host cells (e.g., immune cells such as T cells) that express the fusion proteins and specifically bind to targets (e.g., antigens) that are expressed by or are otherwise associated with a disease or condition, such as, for example, a cancer. In certain aspects, a host cell expressing a fusion protein of the instant disclosure has improved cell signaling (e.g., T cell signaling in a host T cell), proliferation, calcium mobilization, and/or cytotoxic activity in response to target-binding relative to a host cell expressing a reference fusion protein that does not comprise the SH2 domain or functional portion or variant thereof, as disclosed herein. In some embodiments, a host cell of this disclosure (e.g., a T cell) has improved cell signaling upon binding to a target that is expressed at a low level on a target cell (e.g., solid tumor cell) surface.

Also provided are polynucleotides that encode a fusion protein as disclosed herein, vectors that encode a polynucleotide, host cell compositions, and other reagents useful, for example, in treating a disease or disorder such as a cancer. Related methods and uses are also provided.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company. Variant proteins, peptides, polypeptides, and amino acid sequences of the present disclosure can, in certain embodiments, comprise one or more conservative substitutions relative to a reference amino acid sequence.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” refers to a protein that, in a single chain, has at least two distinct domains and/or motifs, wherein the domains or motifs are not naturally found together (e.g., in the given arrangement, order, or number, or at all) in a protein. In certain embodiments, a fusion protein comprises at least two distinct domains and/or motifs that are not found together in a single naturally occurring peptide or polypeptide. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein expressed or produced by a host cell (e.g., a T cell) locates to the cell surface, where the fusion protein is anchored to the cell membrane (e.g., via a transmembrane domain) and comprises an extracellular portion (e.g., containing a binding domain) and an intracellular portion (e.g., containing a signaling domain, effector domain, co-stimulatory domain or combinations thereof).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. If single-stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. In some embodiments, a composition of the present disclosure can be “isolated” in the sense that it is physically separated from and not comprised within a subject to whom the composition can be, was, or is to be administered.

The term “gene” means the segment of DNA involved in producing a polypeptide chain; it includes regions preceding and following the coding region (“leader and trailer”) as well as intervening sequences (introns) between individual coding segments (exons).

A “functional variant” refers to a polypeptide or polynucleotide that is structurally similar or substantially structurally similar to a parent or reference compound of this disclosure, but differs, in some contexts slightly, in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Kₐ) or a dissociation (K_{D}) constant) or avidity; or an assay measuring phosphorylation or activation of, or by, an immune cell protein such as, for example, Lck, ZAP70, Fyn, or the like, including the assays described herein. The ability of a reference polypeptide or encoded polypeptide of this disclosure (or a functional variant of the same) to initiate, continue, participate in, propagate, or amplify a cell signaling event or events (e.g., T cell signaling in response to antigen-binding by a disclosed fusion protein expressed by the T cell) may be determined by examining the activity, structure, chemical state (e.g., phosphorylation), and/or interactions of or between the variant polypeptide and an immune cell protein that directly acts (e.g., binds to or otherwise associates or performs a function) therewith, or by examining the activity, localization, structure, expression, secretion, chemical state (e.g., phosphorylation), and/or interactions of or between other biomolecules known or thought to participate in, or to be affected by, the cell signaling event or events. The ability of a reference polypeptide or encoded polypeptide of this disclosure (or a functional variant of the same) to initiate, continue, participate in, propagate, and/or amplify a cell signaling event or events may also be determined by using functional assays of host cell activity, including those described herein for measuring the ability of a host cell to release cytokines, proliferate, selectively kill target cells, expand and/or persist in vivo, traffic to a target site (e.g., tumor), or treat a subject having a disease or condition expressing or otherwise associated with an antigen or other target bound by a fusion protein of this disclosure.

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release). In certain embodiments, a functional portion refers to a “signaling portion” of an effector molecule, effector domain, costimulatory molecule, or costimulatory domain.

As used herein, the term “SH2 domain” refers to a protein domain that contains a binding or docking site that binds to or docks with a phosphorylated tyrosine residue (pTyr) or a polypeptide comprising the same, but does not bind to or dock with the tyrosine or tyrosine-containing polypeptide when the tyrosine is in an unphosphorylated state. The pTyr is comprised or contained within a peptide or polypeptide that can associate with (e.g., dock with or bind to) the SH2 domain-containing molecule. In certain embodiments, SH2 domains can recognize 3-6 amino acid residues C-terminal to the pTyr in a phosphorylation-dependent manner. Without wishing to be bound by theory, association between an SH2 domain and a pTyr-containing peptide is an important event in signal transduction; e.g., in the response of a T cell to antigen-binding by a TCR.

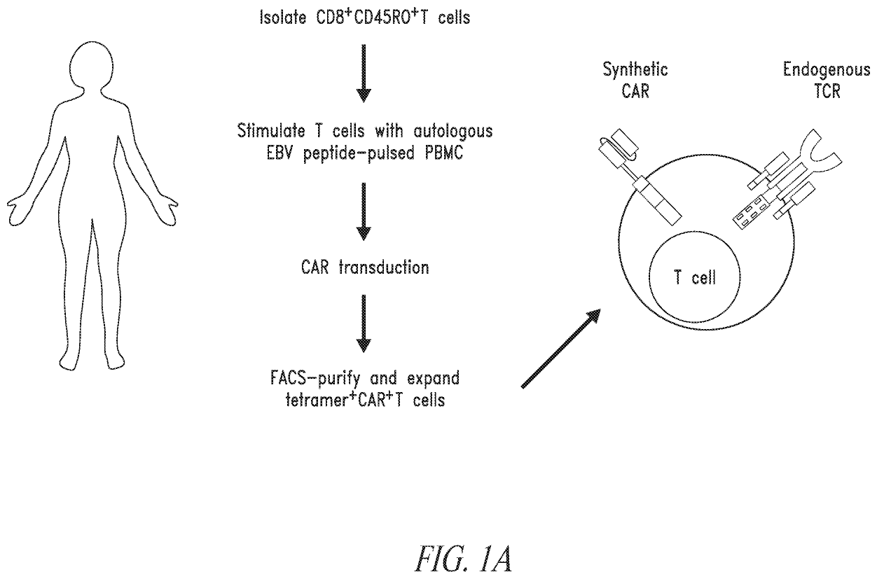

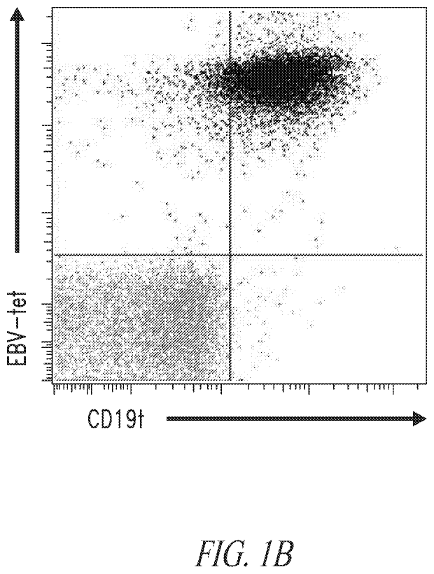

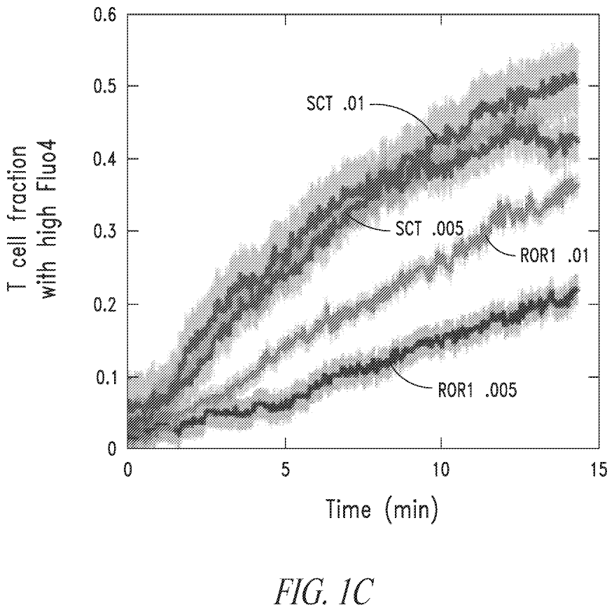

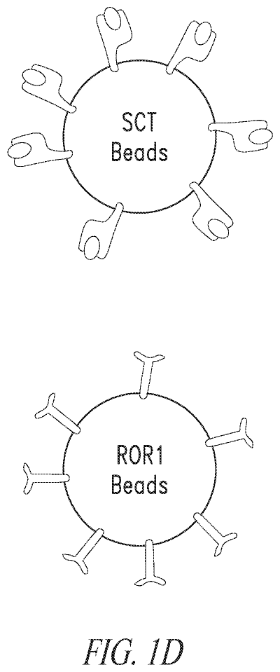

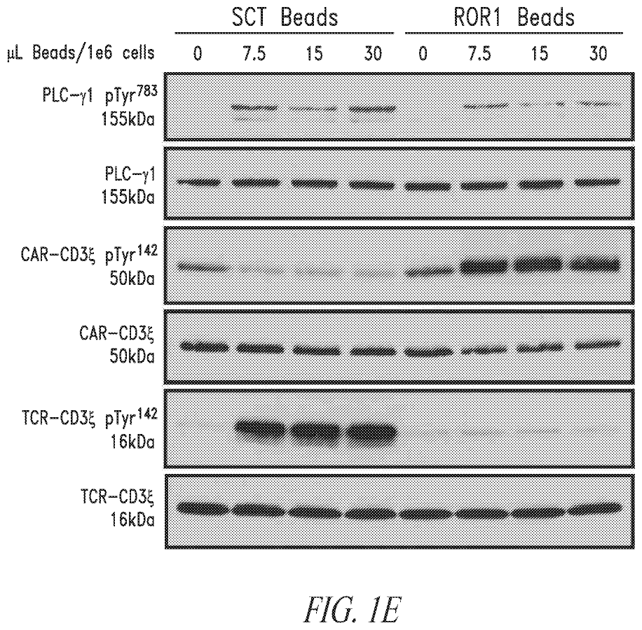

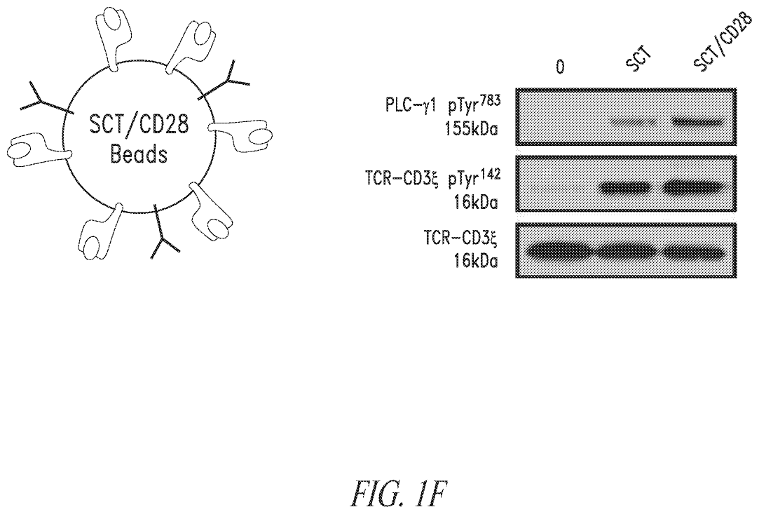

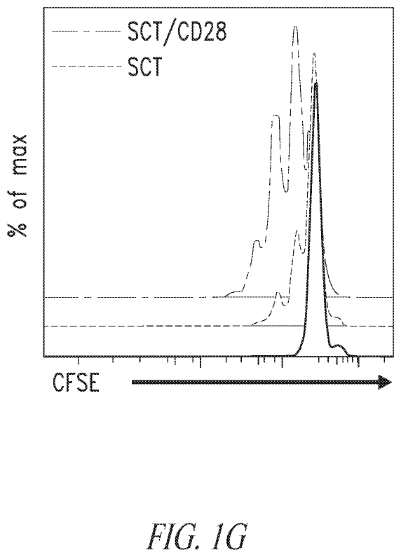

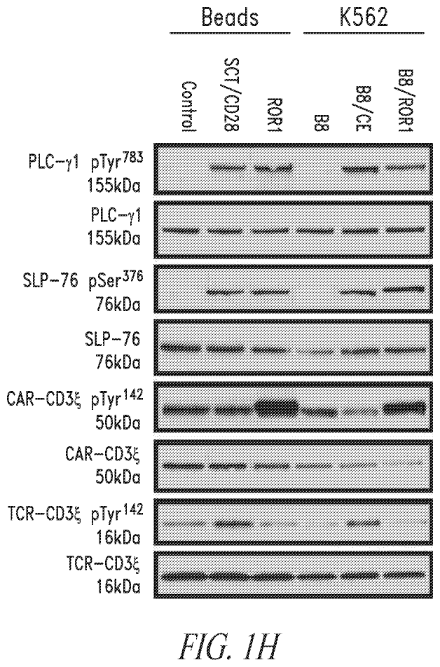

SH2 domains found in nature typically include a secondary protein structure comprising two α-helices and a plurality of β-strands (typically about 6, 7, 8, or more 3-strands). At least some of the β-strands together form a central antiparallel β-sheet, which is flanked on either side by an α-helix. In certain embodiments, a loop region between β-strands 2 and 3 (counting in N to C terminal direction), also referred to as a BC loop, provides binding interactions with the phosphate group of the pTyr. In certain embodiments, amino acids in the first, second, third, and fourth R strands contribute to a binding portion or pocket for the pTyr. Examples of SH2 amino acid positions that are believed to contribute to a pTyr binding pocket are illustrated schematically in FIG. 1 of Kaneko et al., Science Signaling 5(243):ra68 (2012), incorporated herein by reference, and include the amino acids at positions 8, 9, 11, 28, 30, 31, 32, 33, 36, 38, 39, 40, 53, 54, and 56 of SEQ ID NOs.:7-14, and also shown in Figure S4 of Kaneko et al., Science Signaling 5(243):ra68 (2012), which Figure is incorporated herein by reference, and wherein the numbering of those amino acids is according to FIG. 1A in Kaneko et al.

In certain embodiments, amino acids in the SH2 domain N-terminal α-helix and the β-sheet contribute to most or all of the binding interactions with the pTyr-containing amino acid sequence. In certain embodiments, the amino acids in the C-terminal α-helix contribute to a minority or none of the binding interactions with the pTyr-containing amino acid sequence.

SH2 domains of the present disclosure (including functional variants and portions of a reference SH2 domain) are capable of binding to or docking with a pTyr of human LAT (Linker for activation of T-cells family member 1; UniProt KB 043561; SEQ ID NO.:1). In certain embodiments, a LAT Tyr residue that can be phosphorylated includes any of the Tyr residues within positions 28-262 of SEQ ID NO:1, and in particular embodiments, includes a Tyr at any of positions 110, 156, 161, 200, 220, or 255 of SEQ ID NO.:1. In certain embodiments, the 3, 4, 5, or 6 residues of SEQ ID NO.:1 that are immediately C-terminal to the Tyr can comprise a sequence or motif that is recognized by the SH2 domain. In certain embodiments, a SH2 domain of the present disclosure binds to or docks with a LAT amino acid sequence comprising a pTyr at a position corresponding to any of positions 161, 200, or 220 of SEQ ID NO.:1. An exemplary Tyr-containing motif is provided in SEQ ID NO.:2. Exemplary LAT amino sequences that include a Tyr are set forth in SEQ ID NOs.:3-5.

Several human proteins are known to comprise an SH2 domain. In preferred embodiments, an SH2 domain (or functional portion or variant thereof) is from a protein that is involved in T cell receptor signaling, T cell stimulation, and/or T cell differentiation. Exemplary SH2 domains include those from human Grb2, Grap2, Fyn, Src, Grap, CRLK, INPP5D, ITK, LCK, LCP2 aka SLP-76, NKC1, NCK2, PIK3R1, PIK3R2, PLCG1, PLCG2, PTPN6, SH2D1A, SHB, Syk, TEC, VAV1, TXK, ZAP70, BLK, BLNK, BMX, BTK, HSH2D, LYN, PTPN11, SH2B2, SH2D1B, SH2D2A, SH2D3C, SH2D4A, SOCS1, STAT1, STAT2, STAT3, STAT4, STAT5A, STAT5B, STAT6, and YES1.

Also contemplated herein are functional portions and variants of exemplary SH2 domains. Accordingly, the terms “SH2 domain” and “SH2 domain-containing molecule” can also be used to refer, respectively, to a functional portion or a variant of an SH2 domain and to a molecule that comprises the same. A functional portion or functional variant of an SH2 domain retains at least 50% activity associated with the domain, portion or fragment of the parent or reference SH2 domain, preferably at least 55%, 60%, 70%, 75%, 80%, 85%, 90%, 95%, 96%, 97%, 98%, 99%, 99.9%, or 100% level of activity of the parent or reference SH2 domain (e.g., the ability to bind to a LAT pTyr), and/or provides a biological benefit such as, for example, improved LAT clustering in a host cell, improved host cell activation in response to antigen-binding by a host cell antigen-specific receptor, or the like (e.g., the improved function of a SH2-containing fusion protein of this disclosure being in reference to a comparator fusion protein that does not comprise the SH2 domain). Whether two or more proteins form a complex can be determined by any suitable technique, such as, for example, imaging a cell that expresses a detectably labeled (e.g., fluorescently labeled or bound by a labeled antibody) fusion protein of this disclosure and a fluorescently labeled LAT, co-precipitating a fusion protein and a LAT molecule, or the like.

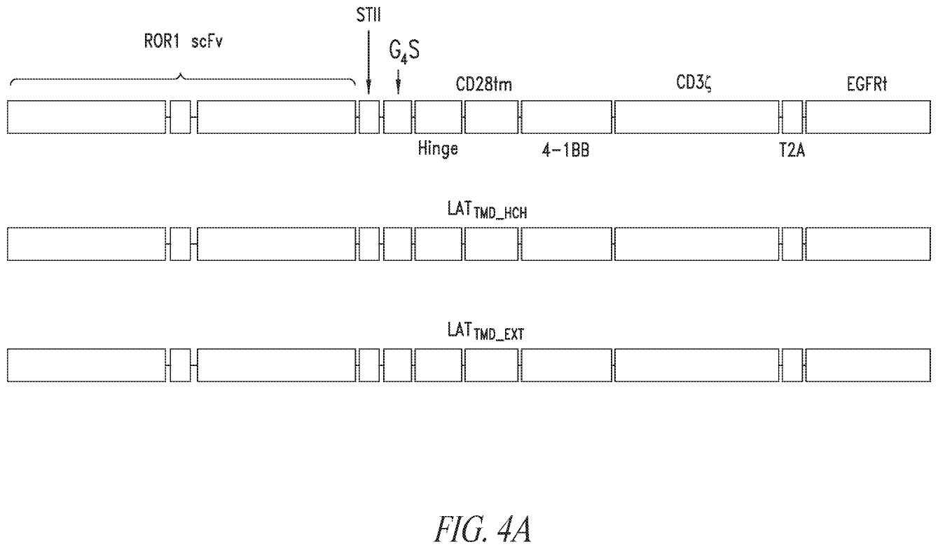

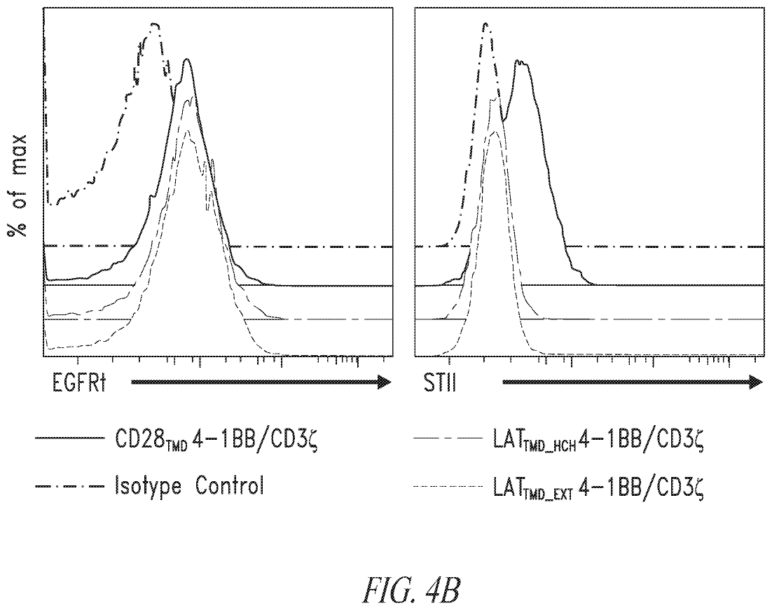

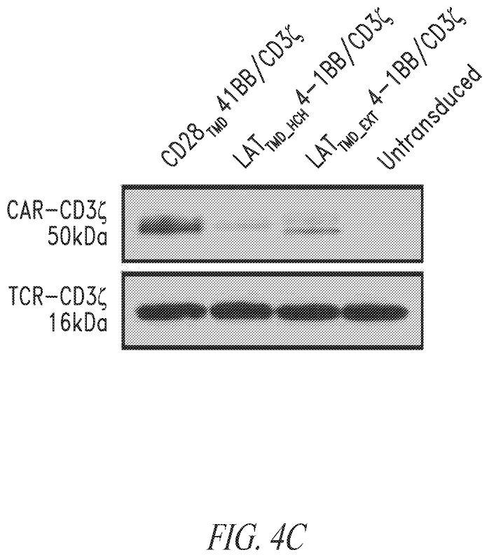

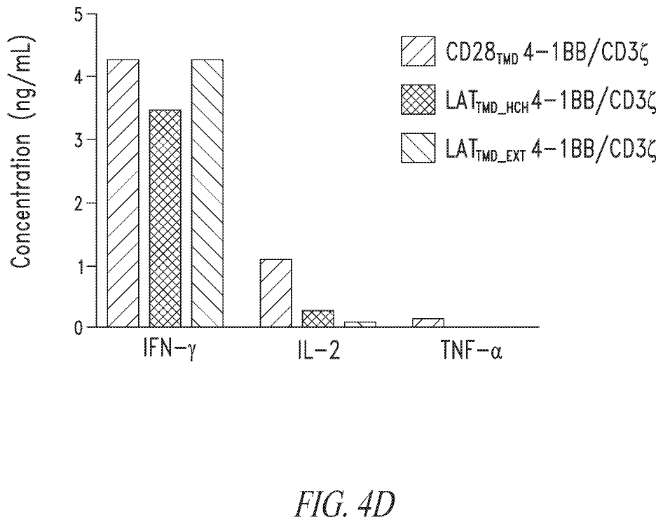

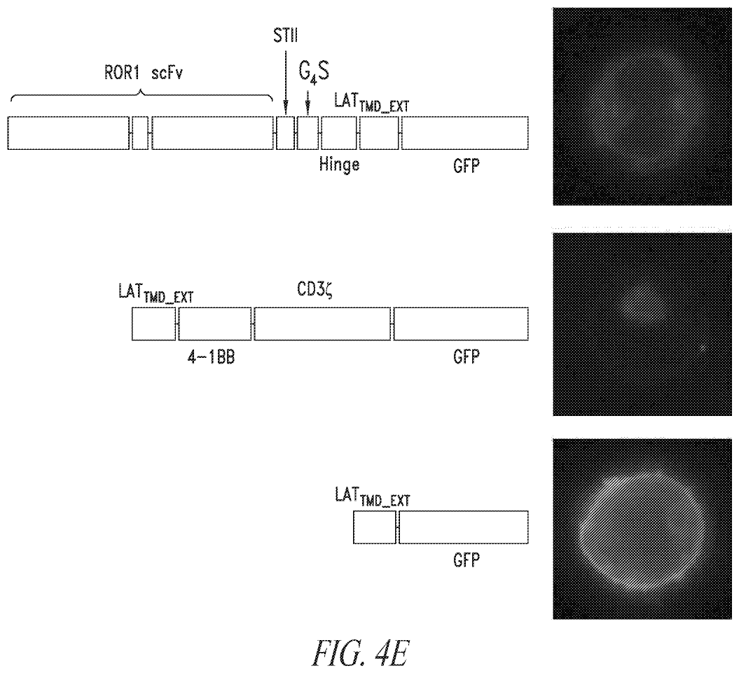

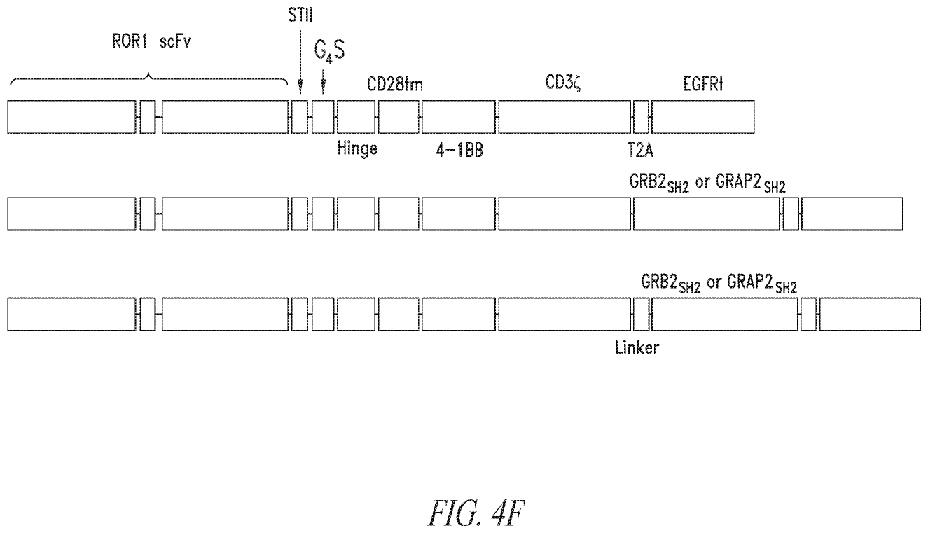

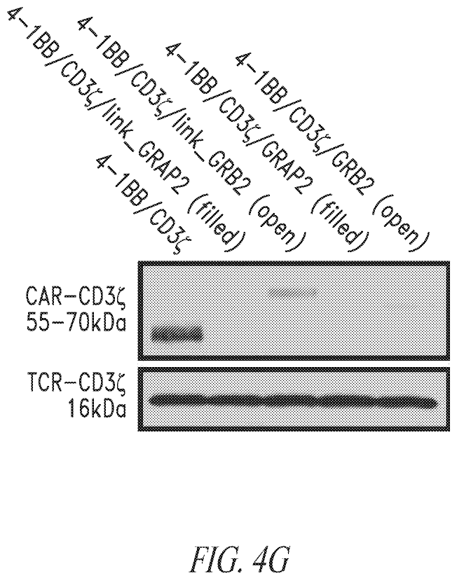

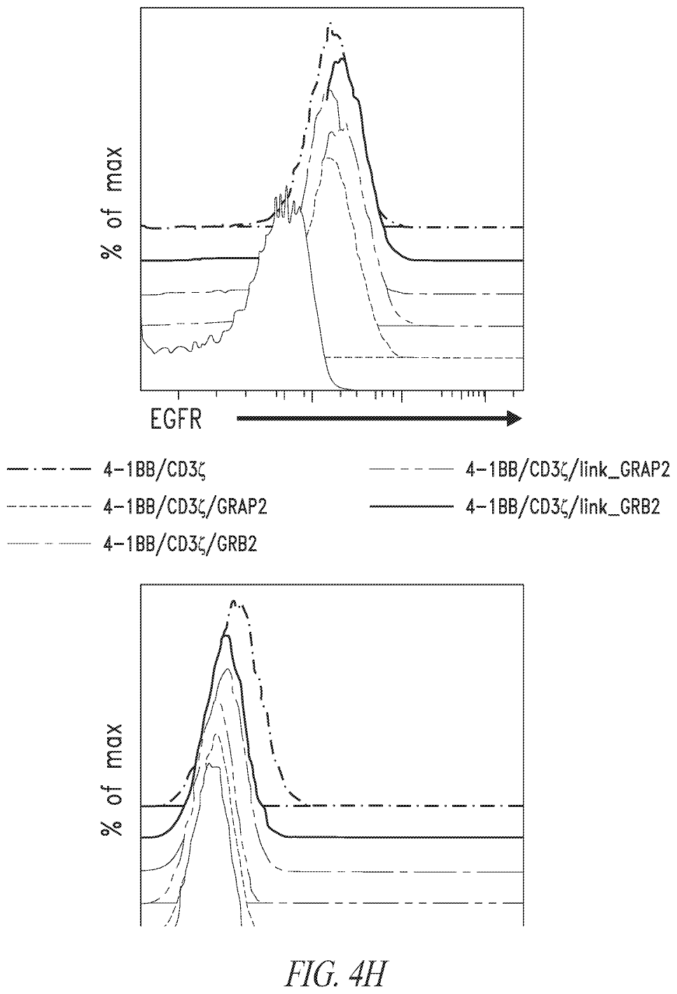

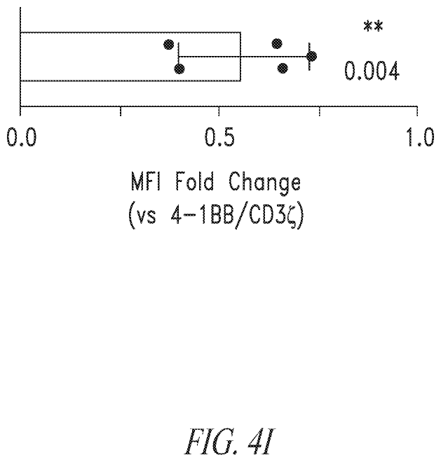

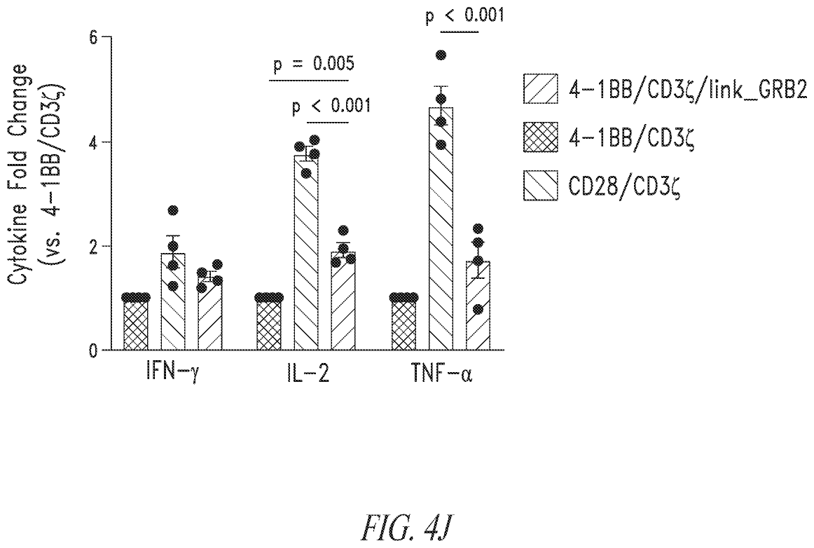

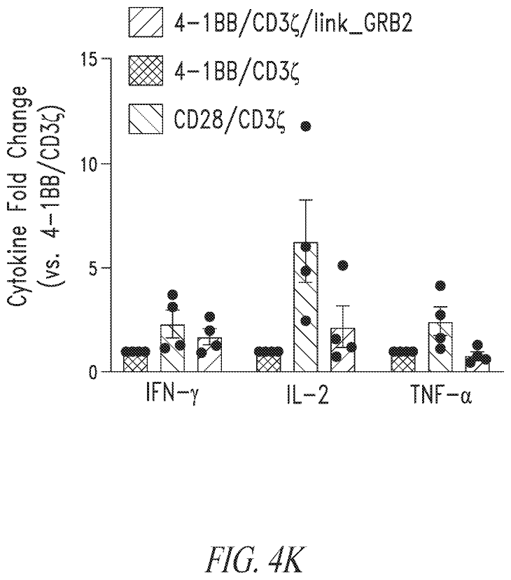

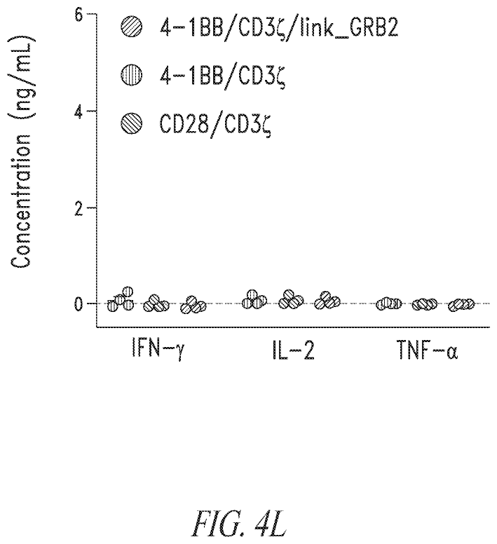

Variant SH2 domains have been described in, for example, Kaneko et al. Science Signaling 5(243):ra68 (2012), and Benfield et al., Arch Biochem Biophys 462(1):47-53 (2007), which variants are incorporated herein by reference. Also contemplated are the variant SH2 domains in PCT Publication No. WO 2013/142965, specifically those which have increased affinity for a pTyr-containing peptide, and including such variants from Grb2, Fyn, or Src (e.g., comprising amino acid substitutions as shown in FIGS. 4, 5, and 7a/7b of WO 2013/142965), such as the Grb2 SH2 variant A8V/S10A/K15L, the Fyn SH2 variant T8V/S10A/K15L, and the Src SH2 variant T8V/C10A/K15L). Accordingly, the variant SH2 domains in WO 2013/142965 are incorporated herein by reference. Contemplated variants include those in which one or more amino acid substitution comprises or is a conservative substitution. Structural aspects of SH2 domains that may be preserved or substantially preserved in a variant have been described (see, e.g., smart.embl.de/smart/do_annotation.pl?DOMAIN=SM00252, which aspects, including a phosphate interacting loop between beta strands 2 and 3; a hydrophobic pocket that can interact with a pTyr+3 amino acids side chain, are incorporated herein by reference).

In certain embodiments, a functional portion or variant of a reference SH2 domain does not comprise a C-terminal amino acid sequence (e.g., up to the C-terminal half) of a SH2 domain, and/or does not comprise a complete or a partial C-terminal alpha helix.

Amino acid sequences of exemplary SH2 domains and functional portions and variants are provided in SEQ ID NOs:7-62.

An SH2 domain, or a functional portion or variant thereof can be, or be derived from, any source, such as a human, a non-human primate, a rodent, an ungulate (e.g., cow, goat, horse, or bison), a rabbit, or another mammal, or from a non-mammalian source (e.g., a yeast; see Dengl et al., J. Mol. Biol. 289(1):211-225 (2009)). In preferred embodiments, a SH2 domain has at least 75%, 80%, 95%, 90%, 91%, 92%, 93%, 94%, 95%, 96%, 97%, 98%, 99%, or more identity to any one of the exemplary human SH2 domains provided herein.

In certain embodiments, an SH2 domain-containing molecule of the present disclosure can facilitate clustering of phosphorylated LAT molecules at the plasma membrane of a host cell (see, e.g., Su et al., Science 352(6285):595 (2016)).

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). It will be appreciated that in the case of a host cell that comprises a heterologous polynucleotide, the polynucleotide is “heterologous” to progeny of the host cell, whether or not the progeny were themselves manipulated to, for example, introduce the polynucleotide.

The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell (e.g., T cell) or microorganism targeted for genetic modification with a heterologous nucleic acid molecule to produce a polypeptide of interest (e.g., a fusion protein of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous TCR; or increased co-stimulatory factor expression).

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

“T cell receptor” (TCR) or “TCR complex” refers to a multi-protein complex known as an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). The extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Ca, typically amino acids 117 to 259 based on Kabat, β-chain constant domain or C_{β}, typically amino acids 117 to 295 based on Kabat) adjacent to the cell membrane. The variable domains contain complementary determining regions (CDRs) separated by framework regions (FRs) (see, e.g., Jores et al., Proc. Nat'l Acad. Sci. U.S.A. 87:9138, 1990; Chothia et al., EMBO J 7:3745, 1988; see also Lefranc et al., Dev. Comp. Immunol. 27:55, 2003). The source of a TCR or TCR binding domain as used in the present disclosure may be from various animal species, such as a human, mouse, rat, rabbit or other mammal.

“CD3” is a multi-protein complex of six chains (see, Abbas and Lichtman, 2003; Janeway et al., p. 172 and 178, 1999). In mammals, the complex generally comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is thought to allow these chains to associate with positively charged regions of T cell receptor chains. The intracellular tails of the CD3 complex proteins contain immunoreceptor tyrosine-based activation motifs or ITAMs, which are thought to be important for T cell signaling in response to antigen binding.

CD3, as well as the protein subunits, domains, and sequences therefrom, may be from various animal species, including human, mouse, rat, or other mammals.

In certain embodiments, a TCR is found on the surface of T cells (also referred to as T lymphocytes) and associates with the CD3 complex. In certain embodiments, a TCR complex comprises a TCR or a functional portion thereof, a dimer comprising two CD3ζ chains, or functional portions or variants thereof, a dimer comprising a CD3δ chain and a CDε chain, or functional portions or variants thereof, and a dimer comprising a CD3γ chain and a CDε chain, or functional portions or variants thereof, any one or more of which may be endogenous or heterologous to the T cell.

“Major histocompatibility complex molecules” (MHC molecules) refer to glycoproteins that deliver peptide antigens to a cell surface. MIIC class I molecules are heterodimers consisting of a membrane spanning a chain (with three a domains) and a non-covalently associated 32 microglobulin. MIIC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. An MIIC molecule may be from various animal species, including human, mouse, rat, cat, dog, goat, horse, or other mammals.

“CD4” refers to an immunoglobulin co-receptor glycoprotein that can assist the TCR in binding to antigen:MHC and communicating with antigen-presenting cells (see, Campbell & Reece, Biology 909 (Benjamin Cummings, Sixth Ed., 2002); UniProtKB P01730). CD4 is found on the surface of immune cells such as T helper cells, monocytes, macrophages, and dendritic cells, and includes four immunoglobulin domains (D1 to D4) that are expressed at the cell surface. During antigen recognition, CD4 is recruited, along with the TCR complex, to bind to different regions of the MHCII molecule (CD4 binds MHCII 02, while the TCR complex binds antigen:MHCII α1/β1).

As used herein, the term “CD8 co-receptor” or “CD8” means the cell surface glycoprotein CD8, either as an alpha-alpha homodimer or an alpha-beta heterodimer. The CD8 co-receptor can assist in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In humans, there are five (5) different CD8 beta chains (see UniProtKB identifier P10966) and a single CD8 alpha chain (see UniProtKB identifier P01732).

“Chimeric antigen receptor” (CAR) refers to a fusion protein engineered to contain two or more amino acid sequences (which may be naturally occurring amino acid sequences) linked together in a way that does not occur naturally or does not occur naturally in a host cell, which fusion protein can function as a receptor (e.g., an antigen-specific receptor) when present on a surface of a cell. CARs of the present disclosure include an extracellular portion comprising a target (e.g., antigen)-binding domain (e.g., obtained or derived from an immunoglobulin or immunoglobulin-like molecule, such as a scFv or scTCR derived from an antibody or TCR (respectively) specific for a cancer antigen, or an antigen-binding domain derived or obtained from a killer immunoreceptor from an NK cell, a designed ankyrin repeat protein (DARPin), an engineered fibronectin type three domain (also referred-to as a monobody) such as an Adnectin™, a ligand (e.g., a cytokine, if the target is a cytokine receptor), a receptor ectodomain (e.g., a cytokine receptor, if the target is a cytokine) or the like) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014)). In certain embodiments, a binding protein comprises a CAR comprising an antigen-specific TCR binding domain (see, e.g., Walseng et al., Scientific Reports 7:10713, 2017; the TCR CAR constructs and methods of which are hereby incorporated by reference in their entirety).

The term “variable region” or “variable domain” refers to the domain of a TCR α-chain or β-chain (or γ-chain and δ-chain for γδ TCRs), or of an antibody heavy or light chain, that is involved in binding to antigen (i.e., contains amino acids and/or other structures that contact antigen and result in binding). The variable domains of the α-chain and β-chain (Vα and Vβ, respectively) of a native TCR generally have similar structures, with each domain comprising four generally conserved framework regions (FRs) and three CDRs. Variable domains of antibody heavy (V_{H}) and light (V_{L}) chains each also generally comprise four generally conserved framework regions (FRs) and three CDRs. In both TCRs and antibodies, framework regions separate CDRs and CDRs are situated between framework regions (i.e., in primary structure).

The terms “complementarity determining region,” and “CDR,” are synonymous with “hypervariable region” or “HVR,” and refer to sequences of amino acids within TCR or antibody variable regions, which, in general, confer antigen specificity and/or binding affinity and are separated from one another in primary structure by framework sequence. In some cases, framework amino acids can also contribute to binding, e.g., may also contact the antigen or antigen-containing molecule. In general, there are three CDRs in each variable region (i.e., three CDRs in each of the TCRα-chain and β-chain variable regions; 3 CDRs in each of the antibody heavy chain and light chain variable regions). In the case of TCRs, CDR3 is thought to be the main CDR responsible for recognizing processed antigen. CDR1 and CDR2 mainly interact with the MHC. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

Fusion proteins of the present disclosure comprise a binding domain that binds to a target. A target can be a molecule expressed on a cell surface, or can be soluble (e.g., a cytokine). In some embodiments, the target is an antigen. “Antigen” or “Ag” as used herein refers to an immunogenic molecule that can provoke an immune response. This immune response may involve antibody production, activation of specific immunologically competent cells (e.g., T cells), secretion of cytokines, or any combination thereof. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a fusion protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease; stabilization of disease state; delay of disease progression; remission; survival; prolonged survival; or any combination thereof. In some embodiments, a benefit of a cellular immunotherapy of this disclosure can further include a reduction (e.g., in number or severity) or absence of a cytokine-related toxicity, such as a cytokine release syndrome.

A “therapeutically effective amount” or “effective amount” of a composition (fusion protein, host cell expressing a fusion protein, polynucleotide, vector, or the like) of this disclosure, refers to an amount of the composition sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. In the case of cancers, benefits can include, for example, a reduction in the size, area, volume, and/or density of a tumor, and/or a reduction or reversal in the rate of tumor growth or spread of cancer,

When referring to an individual active ingredient, administered alone, a therapeutically effective amount refers to the effects of that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient, such as two different antigen-binding proteins (e.g., CARs, TCRs) that specifically bind an antigen, or a fusion protein of the present disclosure.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p-value of 0.050 or less when calculated using the Student's t-test and indicates that it is unlikely that a particular event or result being measured has arisen by chance.

As used herein, the term “adoptive immune therapy” or “adoptive immunotherapy” refers to administration of naturally occurring or genetically engineered, disease-antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

### Fusion Proteins

In certain aspects, the present disclosure provides fusion proteins, wherein the fusion proteins comprise: (a) an extracellular component comprising a binding domain that specifically binds to a target (e.g., an antigen); (b) a transmembrane domain; and (c) an intracellular component comprising an SH2 domain or a functional portion or variant thereof.

A “binding domain” (also referred to as a “binding region” or “binding moiety”), as used herein, refers to a molecule or portion thereof (e.g., peptide, oligopeptide, polypeptide) that possesses the ability to specifically and non-covalently associate, unite, or combine with a target. A binding domain includes any naturally occurring, synthetic, semi-synthetic, or recombinantly produced binding partner for a biological molecule, a molecular complex (i.e., complex comprising two or more biological molecules), or other target of interest. Exemplary binding domains useful in the fusion proteins include single chain immunoglobulin variable regions (e.g., scTCR, scFv, scFab, scTv), Fabs, sdAbs such as nanobodies/VHH, VNAR, receptor ectodomains, ligands (e.g., cytokines, chemokines), or (other) synthetic polypeptides selected for their specific ability to bind to a biological molecule, a molecular complex or other target of interest (e.g., DARPins, ¹⁰FNIII domains). In certain embodiments, the binding domain comprises a scFv, scTv, scTCR, or ligand. In certain embodiments, the binding domain is chimeric, human, or humanized.

In certain embodiments, the binding domain is or comprises a scFv comprising a V_{H} domain, a V_{L} domain, and a peptide linker. In particular embodiments, a scFv comprises a V_{H} domain joined to a V_{L} domain by a peptide linker, which can be in a V_{H}-linker-V_{L} orientation or in a V_{L}-linker-V_{H} orientation.

An scFv may be engineered so that the C-terminal end of the V_{L} domain is linked by a short peptide sequence to the N-terminal end of the V_{H} domain, or vice versa (i.e., (N)V_{L}(C)-linker-(N)V_{H}(C) or (N)V_{H}(C)-linker-(N)V_{L}(C). It will be appreciated that a scTCR or a scTv or a scFab may also be designed in any N-terminal to C-terminal orientation.

As used herein, “specifically binds” or “specific for” refers to an association or union of a binding protein (e.g., a T cell receptor or a chimeric antigen receptor) or a binding domain (or fusion protein comprising the same) to a target molecule with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Binding proteins or binding domains (or fusion proteins thereof) may be classified as “high-affinity” binding proteins or binding domains (or fusion proteins thereof) or as “low-affinity” binding proteins or binding domains (or fusion proteins thereof). “High-affinity” binding proteins or binding domains refer to those binding proteins or binding domains having a Kₐ of at least 10⁷ M⁻¹, at least 10⁸ M⁻¹, at least 10⁹ M¹, at least 10¹⁰ M⁻¹, at least 10¹¹ M⁻¹, at least 10¹²M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, or up to 10⁵ M⁻¹. Alternatively, affinity may be defined as an equilibrium dissociation constant (K_{D}) of a particular binding interaction with units of M (e.g., 10⁻⁵ M to 10⁻¹³ M).

In further embodiments, binding domain of a fusion protein of the instant disclosure can specifically bind to a target antigen (e.g., a cancer antigen such as, for example, a ROR1, CD19, CD20, CD22, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, MAGE-A3, MAGE-A4, SSX-2, PRAME, HA-1, Core Binding Factor (CBF), PSA, ephrin A2, ephrin B2, an NKG2D, NY-ESO-1, TAG-72, mesothelin, NY-ESO, α-fetoprotein, CAR15-3, hCG or beta-hcG, 5T4, BCMA, FAP, Carbonic anhydrase 9, BRAF (such as BRAF^{v600E}) β2M, ETA, tyrosinase, KRAS, NRAS, MR1, or CEA antigen). In some embodiments, a binding domain is capable of specifically binding to an autoimmune antigen, or an antigen that is associated with an infection (e.g., viral, bacterial, fungal, or parasitic).

Sources of binding domains specific for various targets, including antigens as listed above, are known in the art. Exemplary binding domains specific for ROR1 and CD19 antigens, including CDRs thereof, are disclosed SEQ ID NOs.:84-131. A fusion protein of the present disclosure can, in certain embodiments, comprise a variable domain and/or one or more CDRs according to any one of these exemplary binding domain sequences, or can comprise a functional variant sequence thereof.

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof.

A variety of assays are known for identifying binding domains of the present disclosure that specifically bind a particular target, as well as determining binding domain or fusion protein affinities, such as Western blot, ELISA, analytical ultracentrifugation, spectroscopy, isothermal titration calorimetry (ITC), and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for apparent affinity or relative affinity are also known. In certain examples, apparent affinity for a fusion protein is measured by assessing binding to various concentrations of tetramers, for example, by flow cytometry using labeled tetramers. In some examples, apparent K_{D} of a fusion protein is measured using 2-fold dilutions of labeled tetramers at a range of concentrations, followed by determination of binding curves by non-linear regression, apparent K_{D} being determined as the concentration of ligand that yielded half-maximal binding.

In certain embodiments, in addition to the binding domain, the extracellular component of a fusion protein comprises: (i) an immunoglobulin (e.g., IgG, such as IgG1, IgG2, IgG3, or IgG4) CH1 domain, or a functional variant or portion thereof, (ii) an immunoglobulin (e.g., IgG, such as IgG1, IgG2, IgG3, or IgG4) CH2 domain, or a functional variant or portion thereof, (iii) an immunoglobulin (e.g., IgG, such as IgG1, IgG2, IgG3, or IgG4) CH3 domain, or a functional variant or portion thereof, (iv) an immunoglobulin (e.g., IgG, such as IgG1, IgG2, IgG3, or IgG4) CL domain, or a functional variant or portion thereof, (v) a CD8 extracellular domain, or a functional variant or portion thereof; (vi) a CD28 extracellular domain, or a functional variant or portion thereof, (vii) a CD4 extracellular domain, or a functional variant or portion thereof (viii) an IgG (e.g., IgG1, IgG2, IgG3, or IgG4) hinge (e.g., comprising or consisting of the amino acid sequence of SEQ ID NO:71), or a functional variant or portion thereof; (ix) a type II C-lectin interdomain (stalk) region, or a functional variant or portion thereof, (x) a cluster of differentiation (CD) molecule stalk region or a functional variant thereof, (xi) a linker, optionally a glycine-serine linker comprising from about one to about ten repeats of GlyxSery, wherein X and Y are each independently from one to ten (e.g., SEQ ID NOs.:68 and 69, as well as SEQ ID NO.:70); or (xii) any combination of (i)-(xi). In general, the one or more of (i)-(xii) will be disposed between the transmembrane domain and the binding domain. It will be appreciated that a functional variant or portion thereof of a CH1 domain, CH2 domain, CH3 domain, CL domain, CD8 extracellular domain, CD28 extracellular domain, CD4 extracellular domain, type II C-lectin interdomain (stalk) region, cluster of differentiation (CD) molecule stalk region, or IgG hinge (e.g., linkers, as discussed further below) in the context of a fusion protein, is of sufficient length, shape, and/or flexibility to position the binding domain away from the surface of a host cell expressing the fusion protein to enable proper contact between the host cell and a target cell, target (e.g., antigen) binding, and activation of the host cell. In certain embodiments, the extracellular domain comprises a linker disposed between (and optionally, but not necessarily, connecting) the binding domain and the transmembrane domain. In some embodiments, the linker comprises a hinge region or a portion thereof, optionally an IgG hinge amino acid sequence (e.g., SEQ ID NO.:71).

An extracellular component and an intracellular component of a fusion protein of the present disclosure are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In certain embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof), and can be a functional portion or variant thereof; i.e., that retains or substantially retains a three-dimensional structure that is thermodynamically stable in a cell membrane and generally having a length from about 15 amino acids to about 30 amino acids. An exemplary transmembrane amino acid sequence is provided in SEQ ID NO.:72. In certain embodiments, a transmembrane domain comprises or consists of an amino acid sequence having 75% or more, 80% or more, 85% or more, 90% or more, 91% or more, 92% or more, 93% or more, 94% or more, 95% or more, 96% or more, 97% or more, 98% or more, 99%, or 100% identity to the amino acid sequence shown in SEQ ID NO.:28, and optionally having a length comprising or consisting of 35, 34, 33, 32, 31, 30, 29, 28, 27, 26, 25, 24, 23, 22, or 21 amino acids.

In certain embodiments, the extracellular component of the fusion protein further comprises a linker disposed between (and optionally, but not necessarily, connecting) the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the fusion protein to enable proper contact between the host cell and a target cell, target (e.g., antigen) binding, and activation of the host cell (Patel et al., Gene Therapy 6: 412-419, 1999). Linker length may be varied to maximize antigen recognition based on the selected target molecule, selected binding epitope, or antigen binding domain size and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyxSery, wherein x and y are each independently an integer from 0 to 10, provided that x and y are not both 0 (e.g., (Gly₄Ser)₂; (Gly₃Ser)₂; Gly₂Ser; or a combination thereof, such as (Gly₃Ser)₂Gly₂Ser). In some embodiments, the extracellular domain comprises a glycine-serine linker that is not comprised in the binding domain; e.g., is disposed between the transmembrane domain and the binding domain (irrespective of whether the binding domain also comprises such a linker). For example, in certain embodiments, a fusion protein comprises an extracellular domain comprising a first glycine-serine linker disposed between the transmembrane domain and the binding domain, and the binding domain may comprise a scFv or an scTCR or an scTv or an scFab that comprises a second glycine-serine linker, wherein the first and second glycine-serine linkers may be a same or a different glycine-serine linker and may be of a same or a different length. In certain embodiments, a linker has at least about 75% identity to, comprises, consists essentially of, or consists of the amino acid sequence as set forth in any one of SEQ ID NOs:63-71.

Linkers of the present disclosure also include immunoglobulin constant regions (i.e., CH1, CH2, CH3, or CL, of any isotype) and portions and variants thereof. In certain embodiments, the linker comprises a CH3 domain, a CH2 domain, or both. In certain embodiments, the linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105(51):20167 (2008)). In certain embodiments, the linker comprises a human immunoglobulin constant region or a portion thereof. In certain embodiments, the linker comprises an extracellular domain from CD4, or a portion thereof. In some embodiments, the linker comprises an extracellular domain from CD8, or a portion thereof.

In any of the embodiments described herein, a linker may comprise a hinge region or a portion thereof. Hinge regions are flexible amino acid polymers of variable length and sequence (typically rich in proline and cysteine amino acids) and connect larger and less-flexible regions of immunoglobulin proteins. For example, hinge regions connect the Fc and Fab regions of antibodies and connect the constant and transmembrane regions of TCRs. In certain embodiments, the linker comprises an immunoglobulin constant region or a portion thereof and a hinge region or a portion thereof. In certain embodiments, the linker comprises a glycine-serine linker as described herein.

Fusion proteins of the present disclosure also include, comprised in an intracellular component of the fusion protein, an SH2 domain or a functional portion or variant thereof. In certain embodiments, the SH2 domain or functional portion or variant thereof is from Grb2, Grap2, Fyn, Src, Grap, CRLK, INPP5D, ITK, LCK, SLP-76, NKC1, NCK2, PIK3R1, PIK3R2, PLCG1, PLCG2, PTPN6, SH2D1A, SHB, Syk, TEC, VAV1, TXK, ZAP70, BLK, BLNK, BMX, BTK, HSH2D, LYN, PTPN11, SH2B2, SH2D1B, SH2D2A, SH2D3C, SH2D4A, SOCS1, STAT1, STAT2, STAT3, STAT4, STAT5A, STAT5B, STAT6, or YES1.

In further embodiments, the SH2 domain or functional portion or variant thereof comprises or consists of an amino acid sequence having at least about 75% (i.e., at least about 75%, 80%, 85%, 90%, 91%, 92%, 93%, 94%, 95%, 96%, 97%, 98%, 99%, or more) identity to the amino acid sequence shown in any one of SEQ ID NOs.:7-62. In some embodiments, the SH2 domain or functional portion or variant thereof comprises or consists of an amino acid sequence having 75% or more, 80% or more, 85% or more, 90% or more, 91% or more, 92% or more, 93% or more, 94% or more, 95% or more, 96% or more, 97% or more, 98% or more, 99%, or 100% identity to the amino acid sequence shown in any one of SEQ ID NOs.:7-62.

In particular embodiments, the SH2 domain or functional portion or variant thereof comprises or consists of the amino acid sequence set forth in any one of SEQ ID NOs: 7-14. In some embodiments, the SH2 domain or functional portion or variant thereof comprises or consists of the amino acid sequence set forth in SEQ ID NO.:7, 8, or 9.

Certain advantages of presently disclosed fusion proteins can be described, for example, with reference to a host cell that encodes or expresses the same. In any of the presently disclosed embodiments, a fusion protein can be expressed by a host cell (e.g, an immune cell such as a T cell, NK cell, or NK-T cell) and the host cell specifically recognizes and initiates an immune response (e.g., cytotoxic effector function, phagocytosis, antigen-presentation, production of cytokines, production of antibodies, intracellular mobilization of calcium, T cell activation, proliferation of immune cells, or the like) to a target cell expressing a reduced or low or intermediate level or density of the target (e.g., antigen). At such a reduced or low or intermediate level or density of the target, a reference host cell encoding or expressing a reference fusion protein will not initiate the immune response, or will initiate an immune response that is reduced or attenuated in at least one aspect in comparison to the immune response initiated by the host cell expressing a fusion protein of the present disclosure.

A low or intermediate level or density of a target can be in comparison a reference baseline level of expression of the (preferably) same or a different target (e.g., as compared to an average level among subjects or tumors having a same or similar disease or disease state), or to a prior level of expression of the target at a disease site, such as a tumor, in the subject). In certain embodiments, a reduced or low level of expression comprises at a reduction of at least about 5%, 10%, 20%, 30%, 40%, 50%, 60%, 70%, 80%, 90%, 91%, 92%, 93%, 94%, 95%, 96%, 97%, 98%, 99%, or more, as compared to a reference baseline or previous subject level. In certain embodiments, a reduced or low level of expression is a reduction of 1-, 5-, 10-, 20-, 30-, 40-, 50-, 60-, 70-, 80-, 90-, or 100-fold, or more, as compared to a prior or reference level of expression. An expression level of a target (e.g., an antigen) can be determined using any art-accepted methodology, including, for example, use of labeled antibodies, Western Blot, RNA-Seq, or the like. In certain embodiments, a low target (e.g., antigen) density comprises less than about 10,000, less than about 9,000, less than about 8,000, less than about 7,000, less than about 6,000, less than about 5,000, less than about 4,000, less than about 3,000, less than about 2,000, less than about 1,000, less than about 500, less than about 200, less than about 190, less than about 180, less than about 170, less than about 160, less than about 150, less than about 140, less than about 130, less than about 120, less than about 110, about 100, less than about 90, less than about 80, less than about 70, less than about 60, less than about 50, less than about 40, less than about 30, less than about 20, or less than about 10 molecules of the target expressed on the surface of a target cell. In some embodiments, an intermediate target density comprises about 10,000 to about 20,000 molecules of target expressed on the surface of a target cell. In some embodiments, a high target density comprises about 20,000 molecules or more of target expressed on the surface of a target cell.

It will be appreciated that high, intermediate, or low expression is relative and will be understood/determined with respect to the given target and context (e.g., subject, cell, tissue, and/or disease state). For example, a density that is “high” for a given target may be considered “intermediate” or even “low” for a different target, or for the same target in a different subject, cell, tissue, and/or disease state. Reference (e.g., baseline) expression levels can be according to a reference cell, tissue, or population (e.g., subjects of a same gender and/or similar age, ethnic background, or the like, and/or having a same or similar disease at a same or similar state of progression and/or severity), or to the same subject at a different timepoint, or at a different site.

In any of the presently disclosed embodiments, a fusion protein is expressed by a host cell (e.g., an immune system cell such as, for example, a T cell, NK-T cell, or NK cell) and the host cell is capable of producing an increased amount of intracellular calcium (Ca2⁺) in response to the target (e.g., antigen) as compared to a reference host cell expressing a reference fusion protein that does not include the SH2 domain or functional portion or variant thereof. It will be understood that a reference fusion protein can be identical or substantially identical to a fusion protein of this disclosure, with the exception that the SH2 domain or functional portion or variant thereof is not present. In some embodiments, a reference fusion protein can be a fusion protein that comprises one or more different components as compared to a fusion protein according to the present disclosure, but that is also be effective to initiate a response against the target, in at least certain conditions (e.g., high level of target).

By way of illustration, an exemplary fusion protein of the present disclosure may specifically bind to a ROR1 antigen and can comprise a 4-1BB costimulatory domain and a SH2 domain. A reference fusion protein may specifically bind to ROR1 (i.e., at the same or a different epitope, using a same or a different binding domain), and have an identical or substantially identical extracellular domain and transmembrane domain, and can comprise a 4-1BB costimulatory domain, but lack the SH2 domain, or can comprise a CD28 costimulatory domain, but lack the SH2 domain.

A reference host cell will, in general, be a host cell of a same type (e.g., a CD8⁺ T cell as a reference to a CD8⁺ T cell encoding a fusion protein of the present disclosure), and will preferably be phenotypically identical or substantially identical to the host cell of this disclosure, with the exception of the fusion protein and encoding polynucleotide.

In any of the presently disclosed embodiments, a fusion protein is expressed by a host cell (e.g., an immune system cell such as, for example, a T cell, NK-T cell, or NK cell) and the host cell is activated earlier and/or to a greater degree in response to target (e.g., antigen) as compared to a reference host cell expressing a reference fusion protein that does not include the SH2 domain or functional portion or variant thereof.

In any of the presently disclosed embodiments, a fusion protein is expressed by a host cell (e.g., an immune system cell such as, for example, a T cell, NK-T cell, or NK cell) and the host cell has improved anti-tumor activity (e.g., reducing tumor volume, area, growth, or spread; killing tumor cells; preventing tumor growth or proliferation) as compared to a reference host cell expressing a reference fusion protein that does not include the SH2 domain or functional portion or variant thereof.

In any of the presently disclosed embodiments, a fusion protein is expressed by a host cell (e.g., an immune system cell such as, for example, a T cell, NK-T cell, or NK cell) and the host cell has increased phosphorylation of LAT pTyr¹⁹¹ following binding of the fusion protein to target as compared to a reference host cell expressing a reference fusion protein that does not include the SH2 domain or functional portion or variant thereof.

In any of the presently disclosed embodiments, a fusion protein is expressed by a host cell (e.g., an immune system cell such as, for example, a T cell, NK-T cell, or NK cell) and the host cell has decreased production of IL-2 and/or of TNFα in response to target, as compared to a reference host cell expressing a reference fusion protein that does not include the SH2 domain or functional portion or variant thereof.

In any of the presently disclosed embodiments, a fusion protein is expressed by a host cell (e.g., an immune system cell such as, for example, a T cell, NK-T cell, or NK cell) and the host cell increases (i.e., extends) survival in an animal having a cancer (i.e., a cancer expressing or otherwise associated with the target (e.g., antigen) that is specifically bound by the fusion protein) as compared to survival of an animal with the cancer that received a reference host cell expressing a reference fusion protein that does not include the SH2 domain or functional portion or variant thereof.

In any of the presently disclosed embodiments, a fusion protein of the present disclosure can be expressed by a host cell (e.g., an immune system cell such as, for example, a T cell, NK-T cell, or NK cell) at a reduced level, including up to about 2-fold lower, as compared to the expression of a reference fusion protein that does not contain a SH2 domain or functional portion or variant thereof by a reference host cell, and the host cell expressing the fusion protein of the present disclosure has an equivalent or greater response to target (e.g., the host cell mobilizes more intracellular calcium, and/or a greater proportion of host cells expressing the fusion protein mobilize intracellular calcium) as compared to the reference host cell expressing the reference fusion protein. In other words, a fusion protein of the present disclosure can more efficiently initiate a target-specific response as compared to the reference fusion protein, and less of the fusion protein may be needed on a host cell surface in order for the host cell to initiate a more robust response to the target as compared to the response by a reference host cell expressing the reference fusion protein.

In any of the presently disclosed embodiments, a fusion protein is expressed by a host cell (e.g., an immune system cell such as, for example, a T cell, NK-T cell, or NK cell) and the host cell expresses PD-1, LAG3, or both, at a lower level following binding to the target as compared to expression of PD-1, LAG3, or both, in a reference host cell expressing reference fusion protein.

In any of the aforementioned embodiments, a fusion protein and a reference fusion protein can each comprise a 4-1BB and/or a CD28 costimulatory domain, and a CD3ζ effector domain. Constimulatory domains are discussed further herein. In any of the aforementioned embodiments, the host cell expressing the fusion protein and the reference host cell expressing a reference fusion protein are each an immune system cell, optionally a T cell, optionally a CD8⁺ T cell, a CD4⁺ T cell, or both.

In certain embodiments, the intracellular component of the fusion protein comprises an effector domain, or a functional portion or variant thereof.

As used herein, an “effector domain” is an intracellular portion or domain of a fusion protein or receptor that can directly or indirectly promote a biological or physiological response in a cell when receiving an appropriate signal. In certain embodiments, a biological or physiological response is or comprises an immune response. In certain embodiments, an effector domain is from a protein or portion thereof or protein complex that receives a signal when bound, or when the protein or portion thereof or protein complex binds directly to a target molecule and triggers a signal from the effector domain.

An effector domain may directly promote a cellular response when it contains one or more signaling domains or motifs, such as an Intracellular Tyrosine-based Activation Motif (ITAM), such as those found in costimulatory molecules. Without wishing to be bound by theory, it is believed that ITAMs are important for T cell activation following ligand engagement by a T cell receptor or by a fusion protein comprising a T cell effector domain. In certain embodiments, the intracellular component or functional portion thereof comprises an ITAM. Exemplary ITAM consensus amino acid sequences are provided in SEQ ID NOs.:162 and 163; these and other ITAM amino acid sequences are known in the art.

Exemplary effector domains that may be included in a fusion protein of the present disclosure include those from CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMFI, Slp76, pTα, TCRα, TCRβ, TRIM, Zap70, PTCH2, or a functional portion or variant thereof, or any combination thereof. An exemplary CD3ζ intracellular amino acid sequence is provided in SEQ ID NO.:74. In certain embodiments, the effector domain or functional portion or variant thereof comprises or consists of an amino acid sequence having at least about 75% (i.e., at least about 75%, 80%, 85%, 90%, 91%, 92%, 93%, 94%, 95%, 96%, 97%, 98%, 99%, or more) identity to the amino acid sequence shown in SEQ ID NO.:74.

As provided herein, an SH2 domain or functional portion or variant thereof can, in certain embodiments, be disposed N-terminal, or C-terminal, to an effector domain or functional portion or variant thereof. Accordingly, in certain embodiments, an effector domain or functional portion or variant thereof is disposed between the transmembrane domain and an SH2 domain or functional portion or variant thereof. In certain embodiments, an SH2 domain or functional portion or variant thereof is disposed between an effector domain and the transmembrane domain. It will be understood that a component that is “between” two other components can be immediately adjacent to one or both of the two other recited components, or can be separated from one or both of the two other recited components by yet another component or components; thus, “between” refers to a relative position with reference to other enumerated components, and does not necessarily refer to a specific order of components or specific position of a component.

In certain embodiments, the effector domain, or a functional portion or variant thereof, is disposed between the transmembrane domain and the SH2 domain or functional portion or variant thereof.

In certain embodiments, the SH2 domain or functional portion or variant thereof is disposed between the effector domain, or a functional portion or variant thereof, and the transmembrane domain.

In further embodiments, a fusion protein comprises a linker, wherein the linker is disposed between (a) the SH2 domain or functional portion or variant thereof and the effector domain or functional portion or variant thereof and/or (b) the SH2 domain or functional portion or variant thereof and the transmembrane domain. That is, a linker may be disposed on either side of, or on both sides of, the SH2 domain or functional portion or variant thereof, whether the SH2 domain or functional portion or variant thereof is disposed between the effector domain or functional portion or variant thereof and the transmembrane domain, or whether the effector domain or functional portion or variant thereof is disposed between the SH2 domain or functional portion or variant thereof and the transmembrane domain. Any suitable linker may be used, and in general can be about 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human. Exemplary linkers include those having the amino acid sequence set forth in any one of SEQ ID NOs:63-71. In certain embodiments, a linker comprises or consists of an amino acid sequence having at least 75% (i.e., at least about 75%, 80%, 85%, 90%, 91%, 92%, 93%, 94%, 95%, 96%, 97%, 98%, 99%, or more) identity to the amino acid sequence set forth in any one of SEQ ID NOs.:63-71. In some embodiments, a linker comprises or consists of an amino acid sequence having 90%, 91%, 92%, 93%, 94%, 95%, 96%, 97%, 98%, 99%, or 100% identity to the amino acid sequence set forth in any one of SEQ ID NOs.:63-71.

It will be understood that, in addition, one or more linkers may be present elsewhere in the fusion protein.

In certain embodiments, the intracellular component further comprises a costimulatory domain or a functional portion or variant thereof, wherein the costimulatory domain or functional portion or variant thereof is optionally disposed between the effector domain and the transmembrane domain.

In certain embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof. In certain embodiments, the intracellular component comprises a CD28 costimulatory domain or a functional portion or variant thereof (which may optionally include a LL→GG mutation at positions 186-187 of the native CD28 protein (see Nguyen et al., Blood 102:4320, 2003)), a 4-1BB costimulatory domain or a functional portion or variant thereof, or both. Exemplary amino acid sequences of costimulatory domains are provided in SEQ ID NOs.:73 and 75-78. certain embodiments, the costimulatory domain or functional portion or variant thereof comprises or consists of an amino acid sequence having at least 75% (i.e., at least about 75%, 80%, 85%, 90%, 91%, 92%, 93%, 94%, 95%, 96%, 97%, 98%, 99%, or more) identity to the amino acid sequence set forth in any one of SEQ ID NOs:73 and 75-78. In some embodiments, a costimulatory domain or functional portion or variant thereof comprises or consists of an amino acid sequence having 90%, 91%, 92%, 93%, 94%, 95%, 96%, 97%, 98%, 99%, or 100% identity to the amino acid sequence set forth in any one of SEQ ID NOs.:73 and 75-78.

In certain embodiments, an intracellular component of a fusion protein comprises a CD3ζ endodomain or a functional (e.g., signaling) portion thereof, or a functional variant thereof. In certain embodiments, an intracellular component of a fusion protein comprises a CD27 endodomain or a functional (e.g., signaling) portion thereof, or a functional variant thereof. In certain embodiments, an intracellular component of a fusion protein comprises a CD28 endodomain or a functional (e.g., signaling) portion thereof, or a functional variant thereof. In still further embodiments, an intracellular component of a fusion protein comprises a 4-1BB endodomain or a functional (e.g., signaling) portion thereof, or a functional variant thereof. In certain embodiments, an intracellular component of a fusion protein comprises an OX40 endodomain or a functional (e.g., signaling) portion thereof, or a functional variant thereof. In certain embodiments, an intracellular component of a fusion protein comprises a CD2 endodomain or a functional (e.g., signaling) portion thereof, or a functional variant thereof. In certain embodiments, an intracellular component of a fusion protein comprises a CD5 endodomain or a functional (e.g., signaling) portion thereof, or a functional variant thereof. In certain embodiments, an intracellular component of a fusion protein comprises an ICAM-1 endodomain or a functional (e.g., signaling) portion thereof, or a functional variant thereof. In certain embodiments, an intracellular component of a fusion protein comprises a LFA-1 endodomain or a functional (e.g., signaling) portion thereof, or a functional variant thereof. In certain embodiments, an intracellular component of a fusion protein comprises an ICOS endodomain or a functional (e.g., signaling) portion thereof, or a functional variant thereof.

In certain embodiments, the intracellular component of a fusion protein comprises: (i) a costimulatory domain, or a functional portion or variant thereof; (ii) an effector domain, optionally from CD3ζ, or a functional portion or variant thereof; and (iii) an SH2 domain or functional portion or variant thereof, optionally from Grb2 (e.g., having at least about 75% (e.g., 75%, 80%, 85%, 90%, 91%, 92%, 93%, 94%, 95%, 96%, 97%, 98%, 99%, or more) identity to, consisting of, or comprising the amino acid sequence set forth in SEQ ID NO.:7 or 8). In further embodiments, the effector domain is disposed between the costimulatory domain or functional portion or variant thereof and the SH2 domain or functional portion or variant thereof. In other embodiments, the SH2 domain or functional portion or variant thereof is disposed between the costimulatory domain or functional portion or variant thereof and the effector domain or functional portion or variant thereof. In other embodiments, the costimulatory domain or functional portion or variant thereof is disposed between the SH2 domain or functional portion or variant thereof and the effector domain or functional portion or variant thereof. In certain embodiments, the fusion protein further comprises a linker disposed between (and optionally connecting) (i) and (ii), between (ii) and (iii), and/or between (i) and (iii).

In particular embodiments, the intracellular component of the fusion protein comprises, in amino-terminal to carboxy-terminal direction, (i)-(iv): (i) a costimulatory domain (such as, for example, from 4-1BB), or functional portion or variant thereof; (ii) an effector domain from CD3ζ (e.g., which can comprise a CD3ζ endodomain), or a functional portion or variant thereof; (iii) an optional linker; and (iv) an SH2 domain or functional portion or variant thereof from Grb2. In further embodiments, the intracellular domain further comprises a junction amino acid, wherein the junction amino acid is optionally disposed between (i) and (ii), between (ii) and (iii), between (iii) and (iv), or any combination thereof.

In some embodiments, the intracellular component of the fusion protein comprises, in amino-terminal to carboxy-terminal direction, (i)-(iv): (i) a costimulatory domain or functional portion or variant thereof comprising or consisting of an amino acid sequence having at least 90% (i.e., 90%, 91%, 92%, 93%, 94%, 95%, 96%, 97%, 98%, 99%, or more) identity to, comprising, or consisting of, the amino acid sequence set forth in any one of SEQ ID NOs.:73-78; (ii) an effector domain or functional portion or variant thereof comprising or consisting of an amino acid sequence having at least 90% (i.e., 90%, 91%, 92%, 93%, 94%, 95%, 96%, 97%, 98%, 99%, or more) identity to, comprising, or consisting of, the amino acid sequence set forth in SEQ ID NO.:74; (iii) an optional linker, which can comprise the amino acid sequence set forth in any one of SEQ ID NOs.:63-71; (iv) an SH2 domain or functional portion or variant thereof having at least 90% (i.e., 90%, 91%, 92%, 93%, 94%, 95%, 96%, 97%, 98%, 99%, or more) identity to, comprising, or consisting of the amino acid sequence set forth in any one of SEQ ID NOs.:7-62. In some embodiments, the intracellular component of the fusion protein further comprises a further costimulatory domain or functional portion or variant thereof comprising or consisting of an amino acid sequence having at least 90% (i.e., 90%, 91%, 92%, 93%, 94%, 95%, 96%, 97%, 98%, 99%, or more) identity to, comprising, or consisting of, the amino acid sequence set forth in any one of SEQ ID NOs.:73-78.

In certain embodiments, one or more of an extracellular component, a binding domain, a linker, a transmembrane domain, an intracellular component, an effector domain or functional portion or variant thereof, a costimulatory domain or functional portion or variant thereof, or a SH2 domain or functional portion or variant thereof can further comprise one or more junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix) or a fusion protein of this disclosure. Protein tags can be useful for detecting, identifying, isolating, tracking, purifying, enriching for, targeting, or biologically or chemically modifying tagged proteins of interest, particularly when a tagged protein is part of a heterogeneous population of cell proteins or cells (e.g., a biological sample like peripheral blood). In certain embodiments, a protein tag of a fusion protein of this disclosure comprises a Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, Strep tags (e.g., Strep-Tag; Strep-Tag II; and variants thereof, including those disclosed in, for example, Schmidt and Skerra, Nature Protocols, 2:1528-1535 (2007), U.S. Pat. No. 7,981,632; and PCT Publication No. WO 2015/067768, the strep-tag peptides, step-tag-peptide-containing polypeptides, and sequences of the same, are incorporated herein by reference), or any combination thereof.

In any of the embodiments described herein, a fusion protein can be or can comprise a CAR or a TCR. Methods for making fusion proteins, including CARs, are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; Brentjens et al., 2007, Clin. Cancer Res. 13:5426, and Walseng et al., Scientific Reports 7:10713, 2017, the techniques of which are herein incorporated by reference. Methods for producing engineered TCRs are described in, for example, Bowerman et al., Mol. Immunol., 46(15):3000 (2009), the techniques of which are herein incorporated by reference.

In certain embodiments, the TCR comprises a single chain TCR (scTCR), which comprises both the TCR Vα and Vβ domains TCR, but only a single TCR constant domain (Cα or Cβ). In certain embodiments, the antigen-binding fragment of the TCR, or chimeric antigen receptor, is chimeric (e.g., comprises amino acid residues or motifs from more than one donor or species), humanized (e.g., comprises alterations in amino acid sequence from a source non-human protein so as to reduce the risk of immunogenicity in a human), or human.

Methods useful for isolating and purifying recombinantly produced soluble fusion proteins, by way of example, may include obtaining supernatants from suitable host cell/vector systems that secrete the recombinant soluble fusion protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble fusion protein described herein include batch cell culture, which is monitored and controlled to maintain appropriate culture conditions. Purification of the soluble fusion protein may be performed according to methods described herein and known in the art and that comport with laws and guidelines of domestic and foreign regulatory agencies.

Fusion proteins as described herein may be functionally characterized according to any of a large number of art-accepted methodologies for assaying host cell activity. For example, in the case of a host T cell, fusion proteins can be functionally characterized by determination of T cell binding, activation or induction, as well as determination of T cell responses that are target (e.g., antigen)-specific. Examples include determination of T cell proliferation, T cell cytokine release, target-specific T cell stimulation, MHC-restricted T cell stimulation, CTL activity (e.g., by detecting ⁵¹Cr or Europium release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an target-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-γ, IL-12, IL-2, and TNF-β, and Type 2 cytokines, such as IL-4, IL-5, IL-9, IL-10, and IL-13.

### Polynucleotides, Vectors, and Host Cells

In certain aspects, nucleic acid molecules (also referred-to as polynucleotides) are provided that encode any one or more of the fusion proteins as described herein. A polynucleotide encoding a desired fusion protein of this disclosure can be inserted into an appropriate vector (e.g., viral vector or non-viral plasmid vector) for introduction into a host cell of interest (e.g., an immune cell, such as a T cell).

Exemplary markers (e.g., for transduction of a cell with a polynucleotide as provided herein) include green fluorescent protein, an extracellular domain of human CD2, a truncated human EGFR (huEGFRt, (see Wang et al., Blood 118:1255, 2011), a truncated human CD19 (huCD19t); a truncated human CD34 (huCD34t); or a truncated human NGFR (huNGFRt). In certain embodiments, an encoded marker comprises EGFRt, CD19t, CD34t, or NGFRt.

In any of presently disclosed embodiments, a fusion protein-encoding polynucleotide can further comprise a polynucleotide that encodes a marker and a polynucleotide that encodes a self-cleaving polypeptide, wherein the polynucleotide encoding the self-cleaving polypeptide is located between the polynucleotide encoding the fusion protein and the polynucleotide encoding the marker. When the fusion-protein encoding polynucleotide, marker-encoding polynucleotide, and self-cleaving polypeptide are expressed by a host cell, the fusion protein and the marker will be present on the host cell surface as separate molecules. In certain embodiments, a self-cleaving polypeptide comprises a 2A peptide from porcine teschovirus-1 (P2A, Thoseaasigna virus (T2A, equine rhinitis A virus (E2A), or foot-and-mouth disease virus (F2A)). Exemplary nucleic acid and amino acid sequences of 2A peptides are set forth in, for example, Kim et al. (PLOS One 6:e18556, 2011, which 2A nucleic acid and amino acid sequences are incorporated herein by reference in their entirety).

In any of the presently disclosed embodiments, a self-cleaving polypeptide encoded by a chimeric polynucleotide of this disclosure comprises a P2A, a T2A, an E2A, or a F2A. See, e.g., SEQ ID NOs:79-82 and 141-145.

In any of the embodiments described herein, a polynucleotide of the present disclosure (e.g., a fusion protein-encoding polynucleotide or polynucleotide-encoding a a marker) may be codon-optimized for expression in a host cell (see, e.g, Scholten et al., Clin. Immunol. 119:135-145 (2006). Codon optimization can be performed using known techniques and tools, e.g., using the GenScript® OptimumGene™ tool, or the GeneArt™/GeneOptimizer™ tools. Codon-optimized sequences include sequences that are partially codon-optimized (i.e., one or more of the codons is optimized for expression in the host cell) and those that are fully codon-optimized.

In certain embodiments, a polynucleotide encoding a fusion protein of the present disclosure comprises a polynucleotide having at least about 75% identity (i.e., at least about 75%, 80%, 85%, 90%, 91%, 92%, 93%, 94%, 95%, 96%, 97%, 98%, 99%, or more) to the nucleotide sequence set forth in any one of SEQ ID NOs:132-161. In particular embodiments, a polynucleotide encoding a fusion protein comprises a polynucleotide having at least about 75% (i.e., at least about 75%, 80%, 85%, 90%, 91%, 92%, 93%, 94%, 95%, 96%, 97%, 98%, 99%, or more) identity to, or consists of, the nucleotide sequence set forth in any one of SEQ ID NOs:150-161. In further embodiments, a polynucleotide encoding a fusion protein comprises a polynucleotide comprising or consisting of the nucleotide sequence set forth in any one of SEQ ID NOs:152 or 153.

In certain embodiments, polynucleotide encoding a fusion protein of the present disclosure further comprises a polynucleotide encoding a leader or signal sequence. An exemplary leader amino acid sequence is from GM-CSF, which may be encoded by the polynucleotide set forth in SEQ ID NO:165.

In further aspects, expression constructs are provided, wherein the expression constructs comprise a polynucleotide of the present disclosure operably linked to an expression control sequence (e.g., a promoter). An exemplary promoter sequence includes an EF1 promoter according to SEQ ID NO: 164. In certain embodiments, the expression construct is comprised in a vector. An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a vector selected from lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Construction of an expression vector that is used for genetically engineering and producing a fusion protein of interest can be accomplished by using any suitable molecular biology engineering techniques known in the art. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen.

In certain embodiments, polynucleotides of the present disclosure are used to transfect/transduce a host cell (e.g., a T cell). A host cell encoding and/or expressing a fusion protein as disclosed herein is, in certain embodiments, useful in adoptive transfer therapy (e.g., targeting a cancer antigen or targeting an adoptively transferred cell that expresses a tag peptide). Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to fusion proteins of the present disclosure.

In certain embodiments, the host cell is a hematopoietic progenitor cell or a human immune system cell. A “hematopoietic progenitor cell”, as referred to herein, is a cell that can be derived from hematopoietic stem cells or fetal tissue and is capable of further differentiation into mature cells types (e.g., immune system cells). Exemplary hematopoietic progenitor cells include those with a CD24^{Lo} Lin⁻ CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺ T cell, a CD8⁺ T cell, a CD4⁻ CD8⁻ double negative T cell, a γδ T cell, a regulatory T cell, a stem cell memory T cell, a natural killer cell (e.g., a NK cell or a NK-T cell), a B cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs), though it will be understood that a T cell in which expression of a native TCR is (e.g., artificially) suppressed or abrogated is still a T cell. T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}).

Effector T cells (T_{E}) refer to antigen-experienced CD8⁺ cytotoxic T lymphocytes that have decreased expression of CD62L, CCR7, CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells and Treg17 cells, as well as Tr1, Th3, CD8⁺CD28⁻, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells.

In certain embodiments, the immune system cell is a CD4+ T cell, a CD8+ T cell, a CD4− CD8− double negative T cell, a γδ T cell, a natural killer cell (e.g., NK cell or NK-T cell), a dendritic cell, a B cell, or any combination thereof. In certain embodiments, the immune system cell is a CD4+ T cell. In certain embodiments, the T cell is a naïve T cell, a central memory T cell, an effector memory T cell, a stem cell memory T cell, or any combination thereof.

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In any of the foregoing embodiments, a host cell that comprises a heterologous polynucleotide encoding a fusion protein can be an immune cell which is modified to reduce or eliminate expression of one or more endogenous genes that encode a polypeptide product selected from PD-1, LAG-3, CTLA4, TIM3, TIGIT, an HLA molecule, a TCR molecule, or any component or combination thereof.

Without wishing to be bound by theory, certain endogenously expressed immune cell proteins may downregulate the immune activity of a modified immune host cell (e.g., PD-1, LAG-3, CTLA4, TIGIT), or may compete with a heterologous fusion protein of the present disclosure for expression by the host cell, or may interfere with the binding activity of a heterologously expressed fusion protein of the present disclosure and interfere with the immune host cell binding to a target cell that expresses an antigen, or any combination thereof. Further, endogenous proteins (e.g., immune host cell proteins, such as an HLA) expressed on a donor immune cell to be used in a cell transfer therapy may be recognized as foreign by an allogeneic recipient, which may result in elimination or suppression of the donor immune cell by the allogeneic recipient.

Accordingly, decreasing or eliminating expression or activity of such endogenous genes or proteins can improve the activity, tolerance, and persistence of the host cells in an autologous or allogeneic host setting, and can allow universal administration of the cells (e.g., to any recipient regardless of HLA type). In certain embodiments, a modified host immune cell is a donor cell (e.g., allogeneic) or an autologous cell. In certain embodiments, a modified immune host cell of this disclosure comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013) the gene editing techniques, compositions, and adoptive cell therapies of which are herein incorporated by reference in their entirety). As used herein, the term “chromosomal gene knockout” refers to a genetic alteration in a host cell that prevents production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

Alternative Cas nucleases may be used, including but not limited to, Cas 12, Cas 13, and Cas 14 nucleases, and variants thereof. For example, Cas nucleases disclosed in WO 2019/178427, which is hereby incorporated by reference in its entirety (including the Cas nucleases, CRISPR-Cas systems, and related methods disclosed therein), may be utilized.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (e.g., of PD-1, TIM3, LAG3, CTLA4, TIGIT, an HLA component, or a TCR component, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

Any of the foregoing gene-editing techniques can be used to introduce a polynucleotide of the present disclosure (e.g., encoding a fusion protein) into a host cell genome. In some embodiments, a heterologous polynucleotide is introduced into a locus encoding an endogenous TCR component, HLA component, PD-1, LAG-3, CTLA4, TIM3, or TIGIT, or a “safe harbor” locus such as Rosa26, AAVS1, CCR5, or the like.

In certain embodiments, a host cell (e.g., immune cell) of the present disclosure is engineered so that expression of a presently disclosed fusion protein by the host cell is modulated (e.g., controlled) by binding of the host cell to an antigen that is not the same antigen as the antigen to which the fusion protein specifically binds.

For example, a host cell can comprise (i) a polynucleotide encoding an engineered (i.e., synthetic) Notch receptor comprising (a) an extracellular component comprising a binding domain that binds to an antigen, which is a different antigen than the antigen to which the fusion protein binds, (b) a Notch core domain, or a functional portion or variant thereof; and (c) an intracellular component comprising a transcriptional factor (i.e., a polypeptide capable of activating or increasing, or inhibiting, repressing or reducing, transcription of a target nucleotide sequence (e.g., a gene) or set of target nucleotide sequences); and (ii) the heterologous polynucleotide encoding a fusion protein as disclosed herein and comprising an expression control sequence that can be recognized or bound by the transcriptional factor, wherein binding of the engineered Notch receptor to antigen leads to release of the transcriptional factor from the engineered Notch receptor (e.g., by protease-driven cleavage), which can, in turn, drive transcription of the fusion protein. See, e.g., Morsut et al., Cell 164:780-791 (2016) and PCT Published Application No. WO 2016/138034A1, which synthetic Notch constructs are incorporated herein by reference. Briefly, such “logic-gated” expression systems may be useful to modulate expression of a fusion protein of this disclosure so that the expression occurs only, or preferentially, when the host cell encounters a first antigen (i.e., that can be bound by the synthetic Notch receptor) that is only expressed by, or is principally expressed by, or has a higher expression level on cancer cells as compared to healthy cells. Such embodiments may reduce “on-target off-tissue” recognition by a fusion protein in circumstances where the target recognized by the fusion protein is expressed by healthy cells.

In other aspects, kits are provided comprising (a) a vector or an expression construct as described herein and (b) reagents for transducing the vector or the expression construct into a host cell.

### Uses

The present disclosure also provides methods for treating a disease or condition, wherein the methods comprise administering to a subject in need thereof an effective amount of a host cell, composition, or unit dose of the present disclosure, wherein the disease or condition expresses or is otherwise associated with the target (e.g., antigen) that is specifically bound by the fusion protein.

As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like). Certain diseases that involve abnormal or excessive growth that occurs more slowly than in the context of a hyperproliferative disease can be referred to as “proliferative diseases”, and include certain tumors, cancers, neoplastic tissue, carcinoma, sarcoma, malignant cells, pre malignant cells, as well as non-neoplastic or non-malignant disorders.

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

In certain embodiments, a cancer treatable according to the presently disclosed methods and uses comprises a carcinoma, a sarcoma, a glioma, a lymphoma, a leukemia, a myeloma, or any combination thereof. In certain embodiments, cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

In certain embodiments, a cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37+ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

In certain embodiments, the cancer comprises a solid tumor. In some embodiments, the solid tumor is a sarcoma or a carcinoma. In certain embodiments, the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

In certain embodiments, the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma). In certain embodiments, the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

In any of the presently disclosed embodiments, the host cell is an allogeneic cell, a syngeneic cell, or an autologous cell. Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a fusion protein as described herein is administered intravenously, intraperitoneally, intratumorally, into the bone marrow, into a lymph node, or into the cerebrospinal fluid so as to encounter the tagged cells to be ablated. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In any of the above embodiments, methods of the present disclosure comprise administering a host cell expressing a fusion protein of the present disclosure. The amount of cells in a composition is at least one cell (for example, one fusion protein-modified CD8⁺ T cell subpopulation; one fusion protein-modified CD4⁺ T cell subpopulation) or is more typically greater than 10² cells, for example, up to 10⁶, up to 10⁷, up to 10⁸ cells, up to 10⁸ cells, or more than 10¹⁰ cells, such as about 10¹¹ cells/m². In certain embodiments, the cells are administered in a range from about 10⁵ to about 10¹¹ cells/m², preferably in a range of about 10⁵ or about 10⁶ to about 10⁹ or about 10¹⁰ cells/m². The number of cells will depend upon the ultimate use for which the composition is intended as well the type of cells included therein. For example, cells modified to contain a fusion protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁵, 10⁶, 10⁷, 10⁸, 10⁹, 10¹⁰, or 10¹¹ cells.

Unit doses are also provided herein which comprise a host cell (e.g., a modified immune cell comprising a polynucleotide of the present disclosure) or host cell composition of this disclosure. In certain embodiments, a unit dose comprises (i) a composition comprising at least about 30% (e.g., including 30% or more), at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio (e.g., such as a 1:1 ratio), wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of engineered CD45RA⁻ CD3⁺CD8⁺ and engineered CD45RA⁻ CD3⁺CD4⁺ T_{M} cells.

Also contemplated are pharmaceutical compositions that comprise fusion proteins or cells expressing or encoding a fusion protein as disclosed herein, and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the fusion protein-expressing cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with the target (e.g., antigen). Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, fusion protein-expressing T-cell composition to a subject as an adoptive immunotherapy. In certain embodiments, the host cell comprises a hematopoietic progenitor cell or a human immune cell. In certain embodiments, the immune system cell comprises a CD4⁺ T cell, a CD8⁺ T cell, a CD4⁻ CD8⁻ double-negative T cell, a γδ T cell, a natural killer cell, a dendritic cell, or any combination thereof. In certain embodiments, the immune system cell comprises a naïve T cell, a central memory T cell, a stem cell memory T cell, an effector memory T cell, or any combination thereof. In particular embodiments, the cell comprises a CD4⁺ T cell. In particular embodiments, the cell comprises a CD8⁺ T cell.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., fusion protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four weeks or more. In certain embodiments, the plurality of unit doses are administered at intervals between administrations of about two, three, four, five, six, seven, eight, or more weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant.

An effective amount of a pharmaceutical composition (e.g., host cell, fusion protein, unit dose, or composition) refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described fusion proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Target (e.g., antigen)-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a fusion protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁵ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

In further aspects, kits are provided that comprise (a) a host cell, (b) a composition, or (c) a unit dose as described herein.

Methods according to this disclosure may further include administering one or more additional agents to treat the disease or disorder in a combination therapy. For example, in certain embodiments, a combination therapy comprises administering a fusion protein (or an engineered host cell expressing the same) with (concurrently, simultaneously, or sequentially) an immune checkpoint inhibitor. In some embodiments, a combination therapy comprises administering fusion protein of the present disclosure (or an engineered host cell expressing the same) with an agonist of a stimulatory immune checkpoint agent. In further embodiments, a combination therapy comprises administering a fusion protein of the present disclosure (or an engineered host cell expressing the same) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response. For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3, GAL9, KIR, PVR1G (CD112R), PVRL2, adenosine, A2aR, immunosuppressive cytokines (e.g., IL-10, IL-4, TL-iRA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a composition of the present disclosure (e.g., an engineered host cell expressing or encoding a fusion protein as disclosed herein) with one or more inhibitor of any one of the following immune suppression components, singly or in any combination.

In certain embodiments, a composition is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558, or any combination thereof. In further embodiments, composition is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a composition is used in combination with a LAG3 inhibitor, such as LAG525, IMP321, IMP701, 9H12, BMS-986016, or any combination thereof.

In certain embodiments, a composition is used in combination with an inhibitor of CTLA4. In particular embodiments, a composition is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a composition is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a composition is used in combination with an inhibitor of CD244.

In certain embodiments, a composition is used in combination with an inhibitor of BLTA, HVEM, CD160, or any combination thereof. Anti CD-160 antibodies are described in, for example, PCT Publication No. WO 2010/084158.

In certain embodiments, a composition is used in combination with an inhibitor of TIM3.

In certain embodiments, a composition is used in combination with an inhibitor of Gal9.

In certain embodiments, a composition is used in combination with an inhibitor of adenosine signaling, such as a decoy adenosine receptor.

In certain embodiments, a composition is used in combination with an inhibitor of A2aR.

In certain embodiments, a composition is used in combination with an inhibitor of KIR, such as lirilumab (BMS-986015).

In certain embodiments, a composition is used in combination with an inhibitor of an inhibitory cytokine (typically, a cytokine other than TGFβ) or Treg development or activity.

In certain embodiments, a composition is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr. 6-10, 2013), 1-methyl-tryptophan (1-MT)-tira-pazamine, or any combination thereof.

In certain embodiments, a compositionis used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, a fusion protein of the present disclosure (or an engineered host cell expressing the same) is used in combination with an inhibitor of VISTA, such as CA-170 (Curis, Lexington, Mass.).

In certain embodiments, a composition is used in combination with an inhibitor of TIGIT such as, for example, COM902 (Compugen, Toronto, Ontario Canada), an inhibitor of CD155, such as, for example, COM701 (Compugen), or both.

In certain embodiments, a composition is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a composition is used in combination with a LAIR1 inhibitor.

In certain embodiments, a composition is used in combination with an inhibitor of CEACAM-1, CEACAM-3, CEACAM-5, or any combination thereof.

In certain embodiments, a composition is used in combination with an agent that increases the activity (i.e., is an agonist) of a stimulatory immune checkpoint molecule.

For example, a fusion protein of the present disclosure (or an engineered host cell expressing the same) can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a composition with one or more agonist of a stimulatory immune checkpoint molecule, including any of the foregoing, singly or in any combination.

In certain embodiments, a combination therapy comprises a composition and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a composition and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering composition and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2- chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines can be used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same of this disclosure.

In certain embodiments, the subject is receiving, has received, or will receive one or more of: (i) chemotherapy; (ii) radiation therapy; (iii) an inhibitor of an immune suppression component; (iv) an agonist of a stimulatory immune checkpoint agent; (v) RNAi; (vi) a cytokine; (vii) a surgery; (viii) a monoclonal antibody and/or an antibody-drug conjugate; or (ix) any combination of (i)-(viii), in any order.

Also provided herein are uses of any of the presently disclosed fusion proteins, polynucleotides, vectors, host cells, compositions, or unit doses, for use in the treatment of a disease or disorder in a subject, wherein the disease or condition is characterized by the presence of the target that is bound by the binding domain of the fusion protein (e.g., any target as disclosed herein).

Also provided herein are uses of any of the presently disclosed fusion proteins, polynucleotides, vectors, host cells, compositions, or unit doses, for use in the manufacture of a medicament for the treatment of a disease or disorder in a subject, wherein the disease or condition is characterized by the presence of the antigen that is bound by the binding domain of the fusion protein.

### EXAMPLES

### Example 1

### Analysis of TCR and CAR Antigen Sensitivity in a Single Primary T Cell Population

Prior studies have indicated that CAR antigen sensitivity thresholds are significantly higher than those of TCRs, although the mechanisms underlying these differences are unknown (see Sykulev et al., Immunity 4:565-571 (1996); Huang et al., Immunity 39:846-857 (2013); Watanabe et al., J. Immunol. 194:911-920 (2015); Friedman et al., Hum. Gene. Ther. 29:585-601 (2018); Harris et al., J. Immunol. 200:1088-1100 (2018)). To compare TCR and CAR antigen sensitivity in a single cell population, primary human CD8⁺ T cells specific for an epitope of Epstein Barr virus (EBV) presented by HLA-B8 (RAKFKQLL) were derived and engineered to express a CD28/CD3ζ CAR specific for ROR1, which is a tumor-associated antigen being targeted with CAR T cells in patients with advanced ROR1+ cancers (NCT02706392) (FIG. 1A). T cells that both bound HLA-B8/EBV tetramer and expressed the CAR were purified by cell sorting and expanded for analysis (FIG. 1B). These “bi-specific” T cells utilized the endogenous TCR, which avoided potential signaling effects of altered TCR expression or TCRα and β chain mispairing that can occur after transduction of an engineered TCR under non-physiologic regulatory control (see Bendle et al., Nature Med. 16:565-70 (2010); van Loenen et al., PNAS 107:10972-10977 (2010); Terakura et al., Blood 119: 72-82 (2012); Schober et al., Nat. Biomed.

Eng. 7:280 ps7 (2019)).

Fluorescence microscopy using the Ca²⁺-sensitive dye Fluo-4 AM was used to measure Ca²⁺ flux in individual T cells after antigen engagement and provide an index of the relative antigen sensitivity of the TCR and CAR. A recombinant single chain trimer (SCT) consisting of EBV-RAK peptide, HLA-B8, and J2 microglobulin, and a recombinant ROR1 ectodomain, were produced (FIG. 1D). Biotinylated SCT or ROR1 was coated onto a supported lipid bilayer via biotin/streptavidin linkage (see Yu et al., J. Immunol. Nat. Rev. Immunol. 3:939-951 (2003)). Antigen density was modulated by incorporating a small mole fraction (<1%) of biotinylated phycoerythrin (PE) into the supported bilayer, followed by sequential labeling with excess concentrations of streptavidin and biotinylated SCT or ROR1. In these experiments, T cells were exposed to the bilayer and the responses of hundreds of individual cells to TCR or CAR stimulation were compared by calculating the fraction of cells exhibiting high intracellular Ca²⁺ as a metric of the response amplitude. At two low antigen densities, a greater fraction of bi-specific T cells was triggered by TCR stimulation than by CAR stimulation (FIG. 1C). A similar frequency of T cells responded at both SCT antigen densities, consistent with saturation of the TCR response at these low antigen densities. In contrast, the fraction of T cells that responded to ROR1 stimulation did not achieve the level observed with TCR stimulation at the higher antigen density and further declined as ROR1 density was reduced. Because TCR and CAR triggering were quantified at identical SCT and ROR1 densities, these results demonstrated that TCRs possessed increased antigen sensitivity as compared to a clinically relevant CD28/CD3ζ CAR.

### Example 2

### Phosphoproteomic Analysis of TCR and CAR Signaling

To gain insights into the basis for differential antigen sensitivity, TCR and CAR signaling within bi-specific T cells was studied using liquid chromatography coupled with tandem mass spectrometry (LC-MS/MS) after culture of T cells with SCT- or ROR1-coated beads (FIG. 1D). Ligand-coated magnetic beads can initiate CAR signaling without contamination of the cell lysates with antigen presenting cell (APC)-derived proteins (Salter et al., Sci. Signal. 11:eaat6753 (2018)). SCT and ROR1 beads selectively phosphorylated the TCR or CAR, respectively, as well as PLC-γ1 (FIG. 1E). Because CD28/CD3ζ CAR stimulation delivers both ‘signal 1’ and ‘signal 2’, SCT beads were coated with anti-CD28 monoclonal antibody to provide ‘signal 2’ for TCR stimulation. Consistent with the known additive role of CD28 costimulation, SCT/CD28 beads increased PLC-γ1 Tyr⁷⁸³ PO₄ and T cell proliferation after stimulation compared to SCT beads (FIGS. 1F and 1G) (see Acuto & Michel, Nat. Rev. Immunol. 3:939-951 (2003)). To further validate a bead-based approach, PO₄ of key signaling intermediates in bi-specific T cells was compared after 45 minutes of stimulation with K562 APC or ligand-coated beads. PO₄ of native CD3ζ or exogenous CAR-CD3ζ chains at Tyr¹⁴² appeared nearly identical after antigen presentation by APC or beads, and PO₄ of SLP-76 Ser³⁷⁶ and PLC-γ1 Tyr⁷⁸³ was highly similar (FIG. 1H). Bead stimulation was therefore used for subsequent comparisons of TCR and CAR signaling.

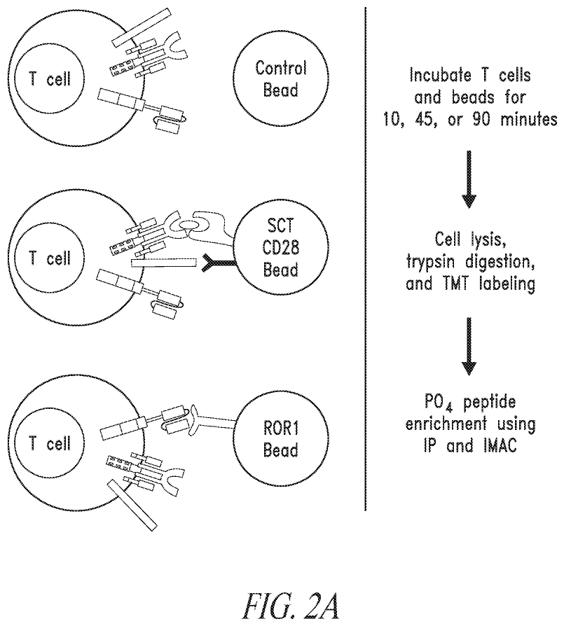

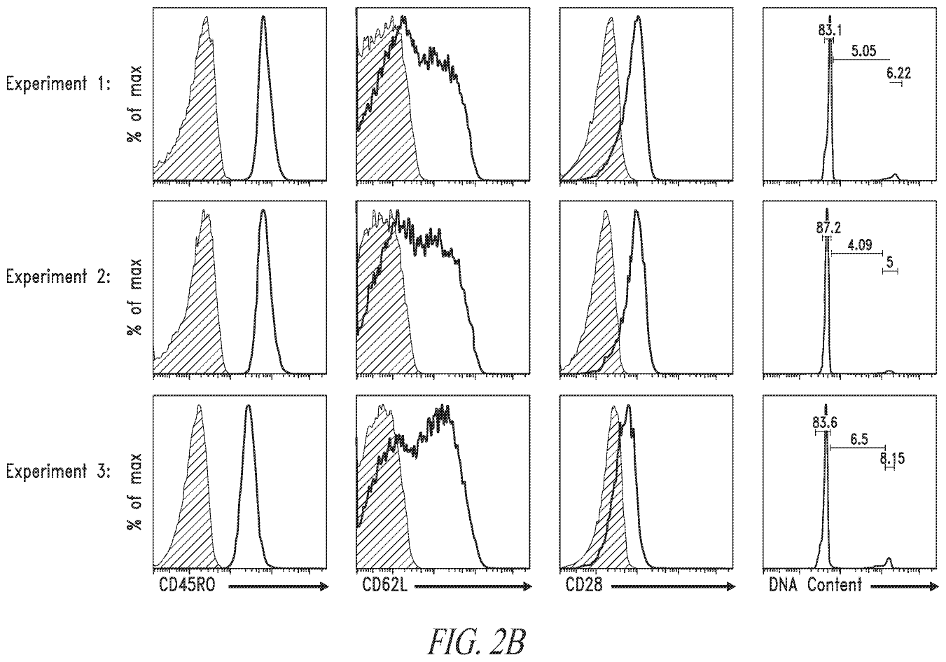

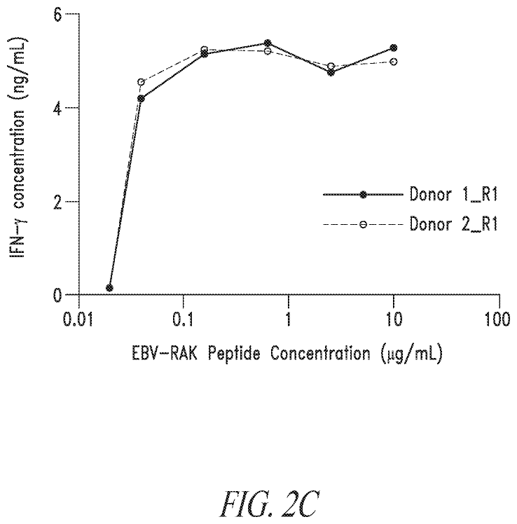

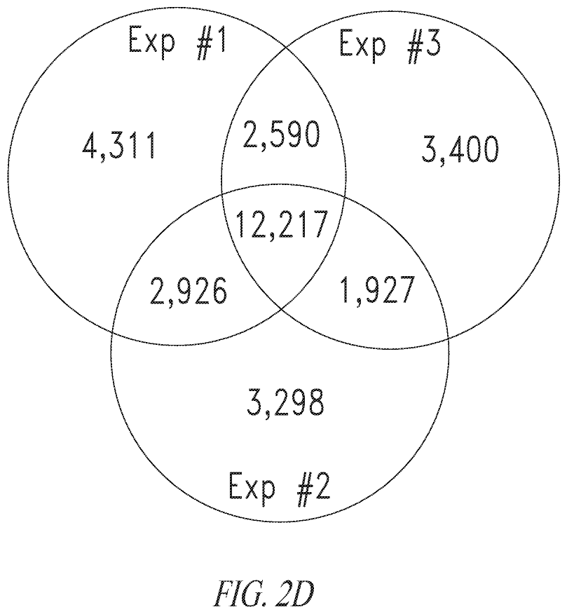

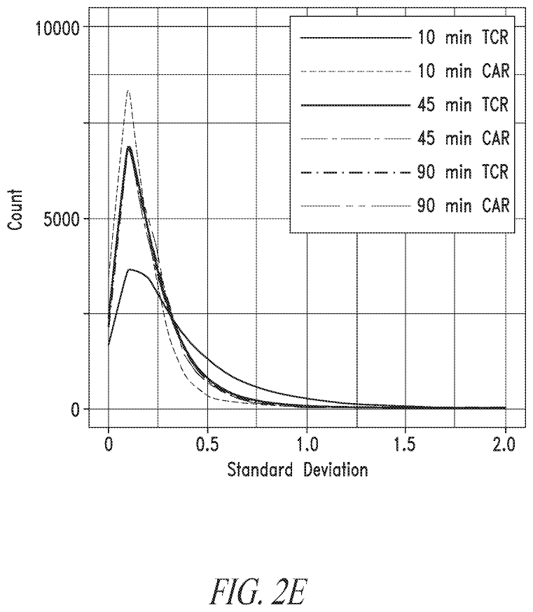

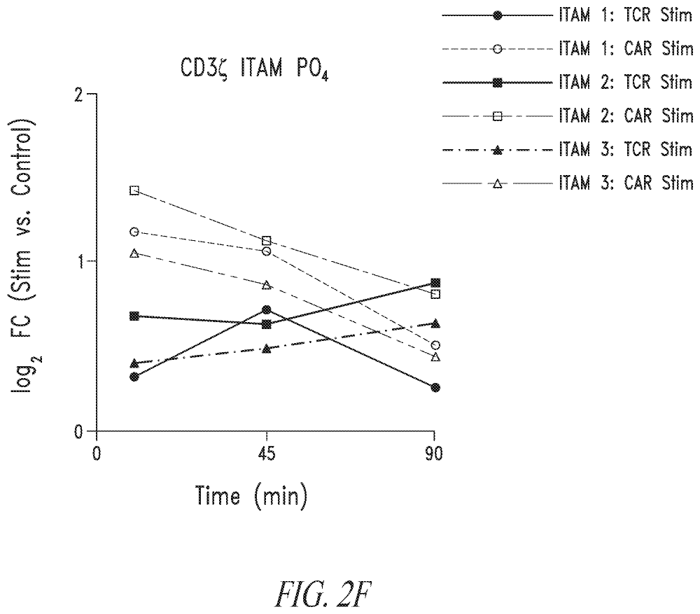

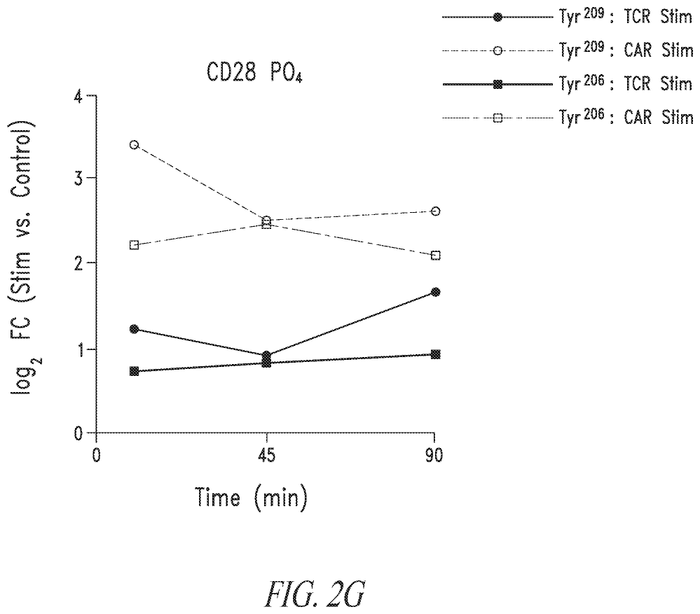

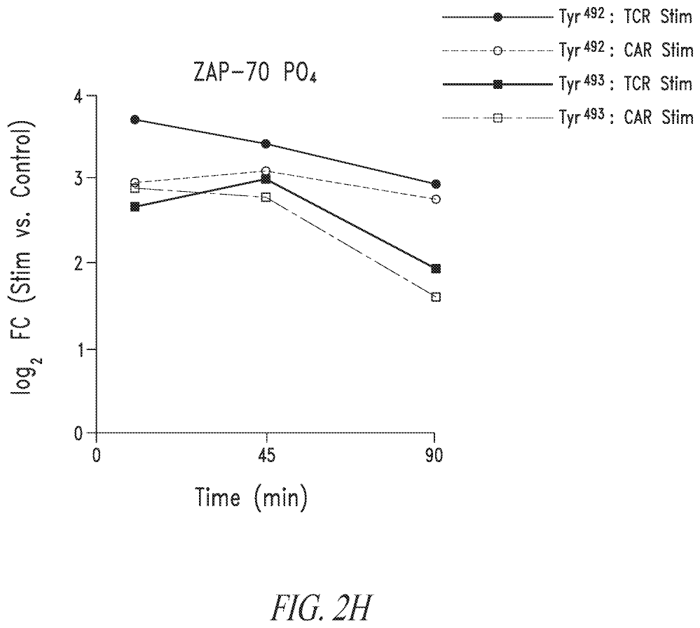

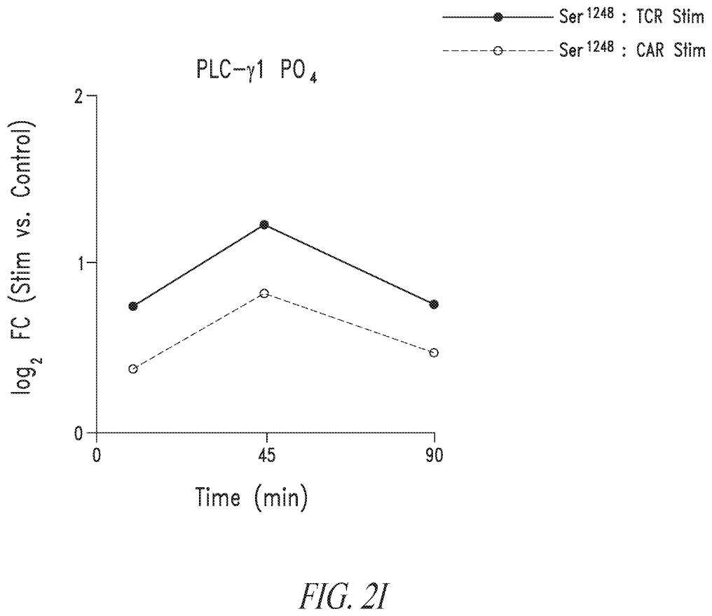

Cell lysates for LC-MS/MS analysis were prepared after incubating bi-specific T cells with SCT/CD28 (TCR), ROR1 (CAR), or uncoated (control) magnetic beads for 10, 45, or 90 minutes (FIG. 2A). Three independent experiments were performed using cells isolated from two donors. Cell phenotyping prior to stimulation showed that the T cells from each donor uniformly expressed CD45RO, a majority expressed CD62L and CD28, and >83% were in the G₀/G₁ cell cycle phase, indicating the cells had returned to a quiescent memory phenotype after transduction and expansion (FIG. 2B). Recognition of K562/HLA-B8 APC pulsed with titrated amounts of peptide indicated the functional avidity of the EBV-specific T cells from each of the donors was similar (FIG. 2C). A total of 30,669 PO₄ sites corresponding to 4,997 gene products were detected in the MS dataset, with 64% of PO₄ sites observed in two or three experiments (FIG. 2D). 715 PO₄ sites (2.3%) were phosphotyrosines, 5,056 (16.5%) were phosphothreonines, and 24,898 (81.2%) were phosphoserines. Log₂FC values comparing TCR stimulation to control treatment or CAR stimulation to control treatment were similar across replicate experiments (FIG. 2E).

### Example 3

### Differential Phosphorylation of T Cell Signaling Proteins by CAR Vs. TCR Stimulation

Differences in protein PO₄ between CAR and TCR stimulation were examined by analyzing the log 2FC of PO₄ sites in TCR- and CAR-stimulated samples relative to control samples. Query of the dataset for PO₄ sites on CD3ζ, CD28, ZAP-70, and PLC-71 confirmed that these canonical signaling proteins were phosphorylated after both TCR and CAR engagement (see Brownlie & Zamyoska, Nat. Rev. Immunol. 13:257-269 (2013)). All three CD3ζ ITAMs, as well as CD28 Tyr²⁰⁶ and Tyr²⁰⁹, were more intensely phosphorylated by CAR stimulation than TCR stimulation at the 10- and 45-minute time points, despite no mechanism for recruiting the CD8 co-receptor and its associated pool of Lck by CAR binding to recombinant ROR1 (FIGS. 2F and 2G) (see Artyomov et al. PNAS U.S.A. 107:16916-16921 (2010)). Lck directly associates with CARs (see Salter et al. (supra) and Rohrs et al. Biophys. J. 115:1116-1129 (2018)), which may account for the greater CD3ζ and CD28 PO₄. Despite stronger CD3ζ PO₄ after CAR engagement, ZAP-70 Tyr⁴⁹³ displayed a similar log 2FC in PO₄ after TCR and CAR stimulation, and PLC-γ1 Ser¹²⁴⁸ was more weakly phosphorylated by CAR than TCR stimulation (FIGS. 2H and 2I). These findings were surprising, since the more intense CD3ζ and CD28 PO₄ after CAR stimulation might be expected to result in increased PLC-γ1 PO₄.

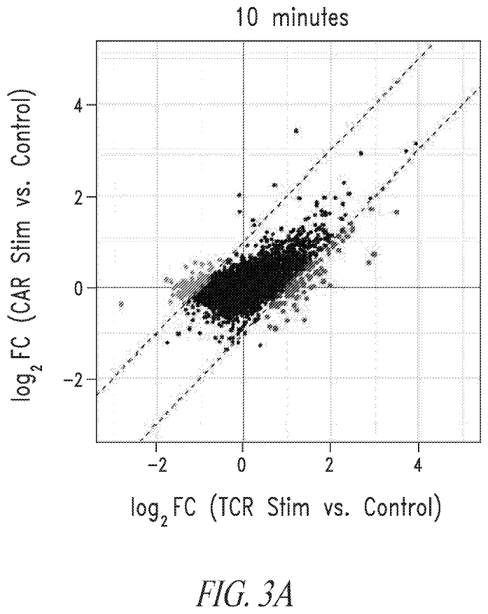

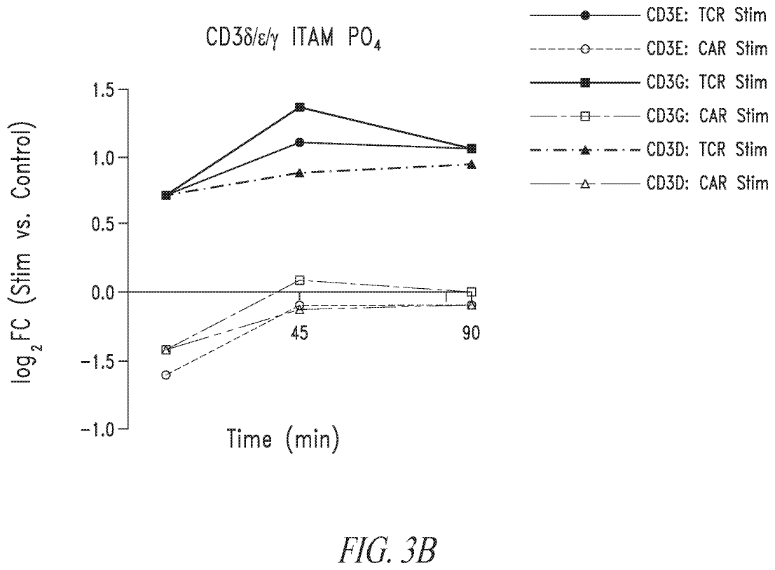

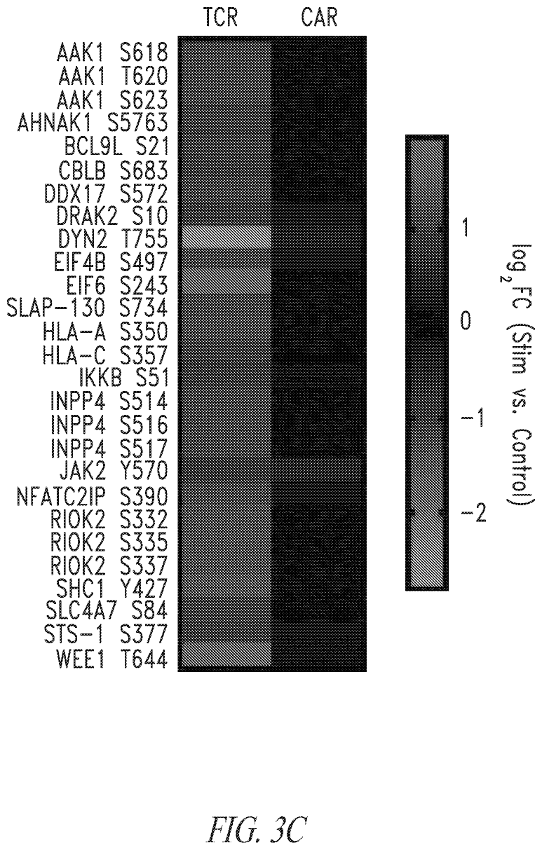

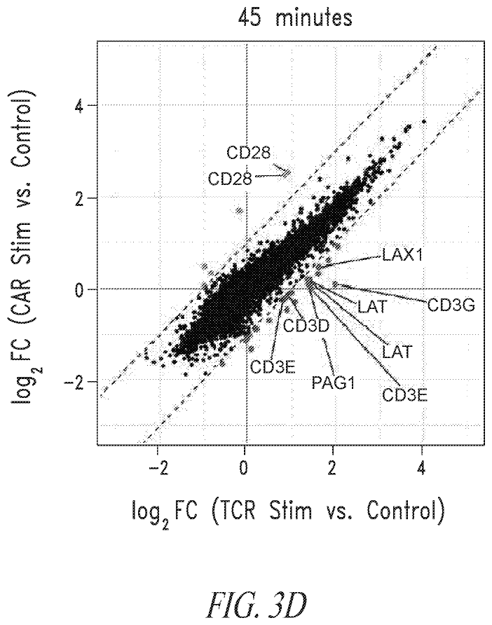

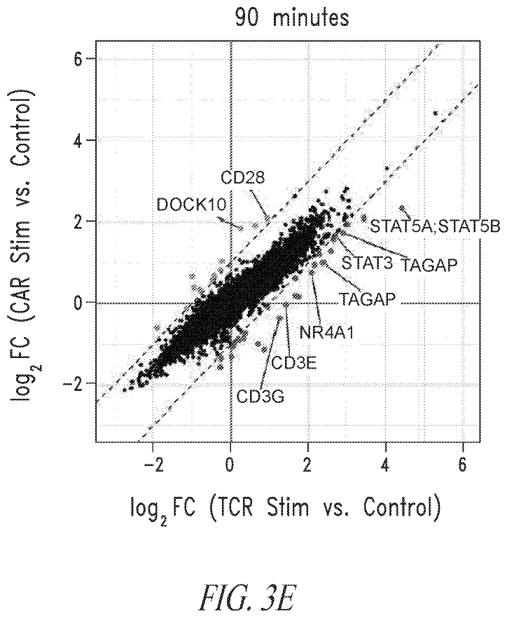

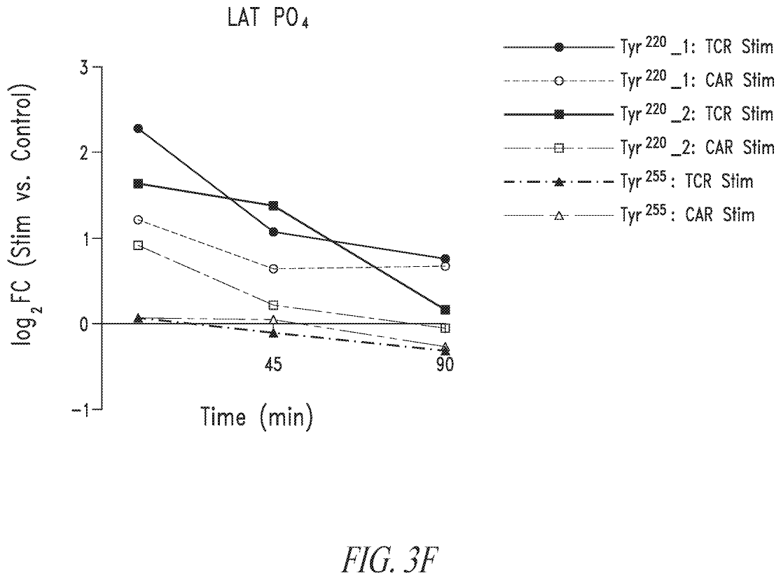

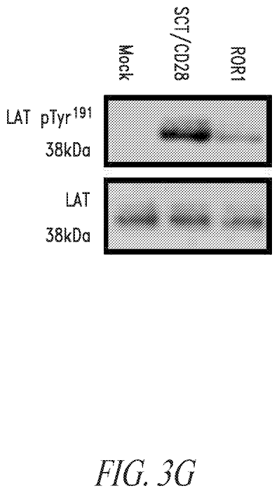

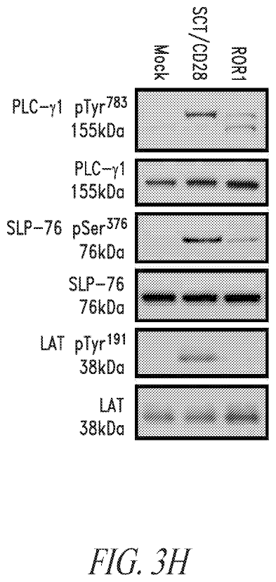

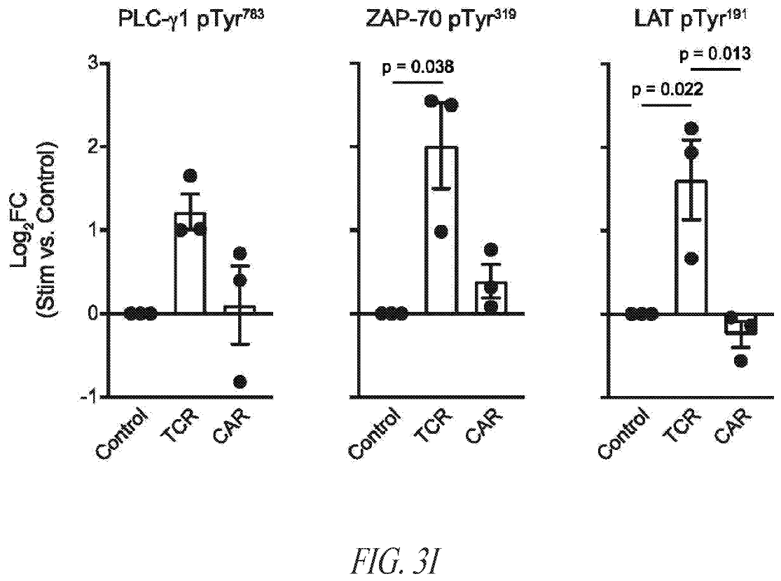

At the 10-minute time point, a weak association between TCR- and CAR-stimulation-induced changes in protein PO₄ was observed. Filtering of the data for PO₄ sites that differed by at least 2-fold between TCR- and CAR-stimulated samples revealed that 236 PO₄ sites were more intensely modulated by TCR than CAR stimulation (FIG. 3A). It was notable that at 10 minutes, the ITAMs on CD3δ, CD3ε, and CD3γ were phosphorylated by TCR stimulation and de-phosphorylated by CAR stimulation (FIG. 3B). Signaling intermediates (AHNAK, CBLB, IKBKB, INPP4A, RAPGEF1, RAPGEF2 and SHC1), as well as RNA binding proteins (DDX17, EIF6, EIF4B, and RBM25), were also phosphorylated by TCR stimulation but not by CAR stimulation (FIG. 3C).

At the 45-minute time point, TCR- and CAR-stimulated samples exhibited greater concordance and only 30 PO₄ sites possessed fold change values that differed >2-fold between TCR and CAR stimulated samples (FIG. 3D). Specifically, tyrosine residues on positive regulators of T cell signaling such as CD3δ, CD3ε, CD3γ, and LAT, as well as negative regulators such as LAX1 and PAG1 were more intensely phosphorylated by TCR than CAR stimulation. LAT and PAG1 had displayed slight (<2-fold) differences in protein PO₄ at the 10-minute time point that did not meet the cutoffs, indicating that certain differences in proximal signaling after CAR and TCR engagement were magnified over time. After 90 minutes, 41 sites possessed fold change values that differed by more than 2-fold between TCR and CAR stimulated samples (FIG. 3E). Tyrosine residues in CD3ε and CD3γ ITAMs remained more intensely phosphorylated by TCR-than CAR-stimulation, and preferential PO₄ of TAGAP, SOS1, STAT3, STAT5A/B, and NR4A1 was detected after TCR, but not CAR stimulation.

The finding that LAT-a critical signaling hub- and other important signaling intermediates were less intensely phosphorylated by CAR stimulation than TCR stimulation was not previously appreciated (FIG. 3F). To validate the early difference in LAT PO₄, lysates from bi-specific T cells stimulated with SCT/CD28 or ROR1 beads were analyzed by Western blot. Consistent with the LC-MS/MS data, weaker LAT Tyr¹⁹¹ PO₄ was observed after 10 minutes of CAR stimulation than TCR stimulation (FIG. 3G). These data suggest that despite rapid and intense CAR-CD3ζ PO₄ after CAR ligand binding, PO₄ of downstream signaling intermediates is delayed and/or weaker than following TCR stimulation, with potential consequences for the antigen threshold necessary to elicit effector activation.

A CD28/CD3ζ CAR was utilized for LC-MS/MS analyses to facilitate direct comparisons to TCR/CD28 stimulation. T cells engineered with CARs containing 4-1BB/CD3ζ signaling domains are also used in cancer therapy, and 4-1BB/CD3ζ CAR T cells may persist longer and promote less severe cytokine release syndrome than CD28/CD3ζ CAR T cells (see Shultz & Mackall, Sci. Transl. Med. 11:eaaw2127 (2019)). To investigate whether 4-1BB/CD3ζ CAR stimulation yielded similar differences in protein PO₄ from TCR stimulation, bi-specific T cells expressing an otherwise structurally identical ROR1-specific 4-1BB/CD3ζ CAR were stimulated with SCT/CD28 or ROR1 beads for 10 minutes. Western blot analysis of cell lysates confirmed that TCR stimulation induced more intense PO₄ of LAT, SLP-76 and PLC-71 than 4-1BB/CD3ζ CAR stimulation (FIGS. 3H and 3I). Thus, compared to TCR stimulation, CD28/CD3ζ and 4-1BB/CD3ζ CARs both displayed defects in SLP-76 and PLC-γ1 PO₄ after stimulation.

### Example 4

### Inclusion of an SH2 Domain Enhances CAR Antigen Sensitivity

Since CAR stimulation also induced a lower level of LAT PO₄, CAR designs were investigated that might improve LAT PO₄ and potentially augment the antigen sensitivity of 4-1BB/CD3ζ CAR T cells. First, encoding the LAT transmembrane domain as a part of the CAR construct was evaluated to determine whether this would localize the CAR to lipid rafts and improve LAT binding. However, this approach was deemed unsuccessful because 4-1BB/CD3ζ CARs containing two unique LAT_{TMD} variants that differed in length were not well-expressed on the surface and in cell lysates (FIGS. 4A-4C) (see Zhang et al., Immunity 9:239-246 (1998)). Consistent with this cell surface expression, LAT_{TMD} CAR T cells only produced IFN-γ and failed to secrete IL-2 and TNF-α after CAR stimulation (FIG. 4D). Analysis of CAR-LAT_{TMD}-GFP fusion proteins revealed that a LAT_{TMD}-GFP fusion protein lacking CAR structural elements could be expressed on the T cell surface at high levels, but GFP fusion proteins containing either the extracellular or intracellular CAR sequences were weakly expressed and existed in intracellular compartments (FIG. 4E).

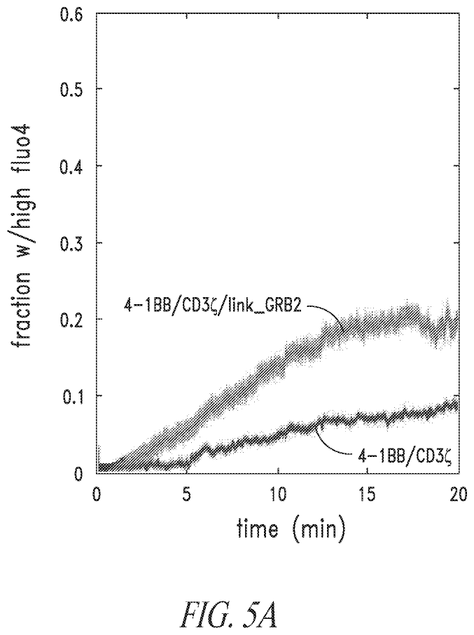

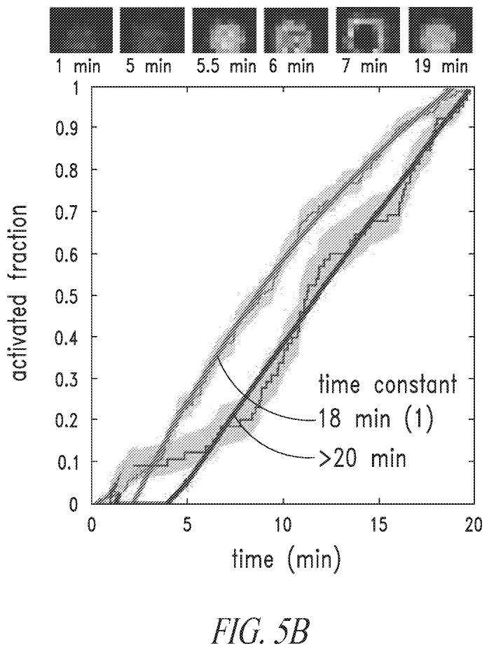

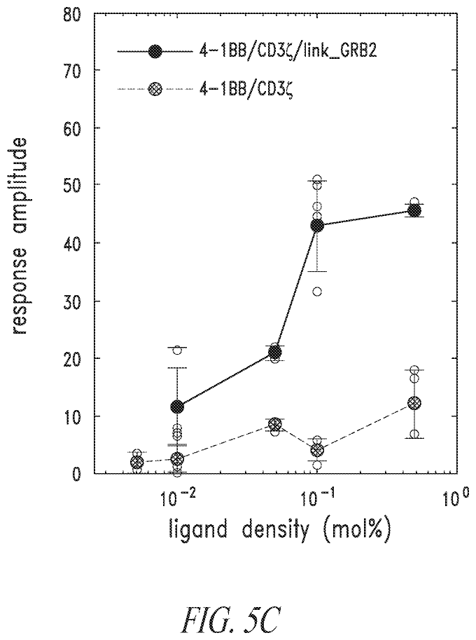

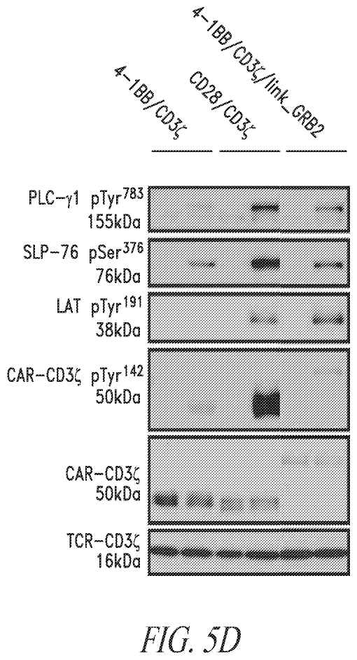

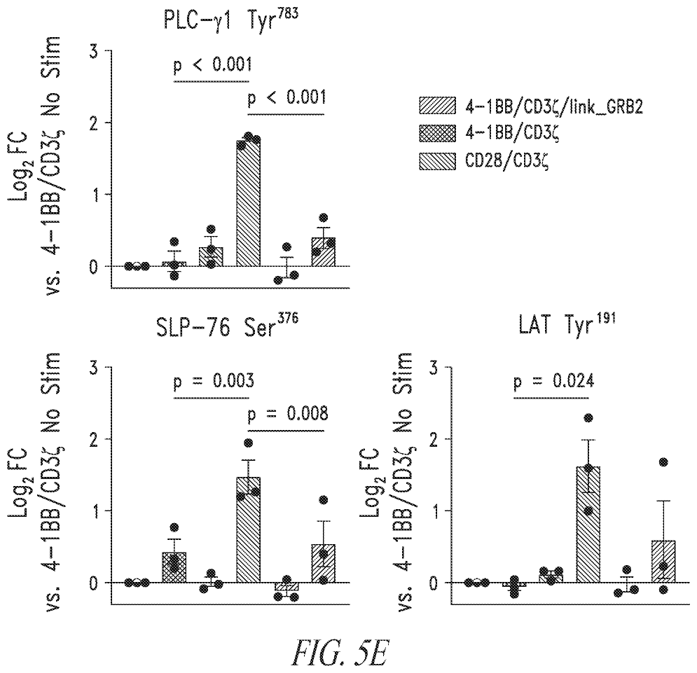

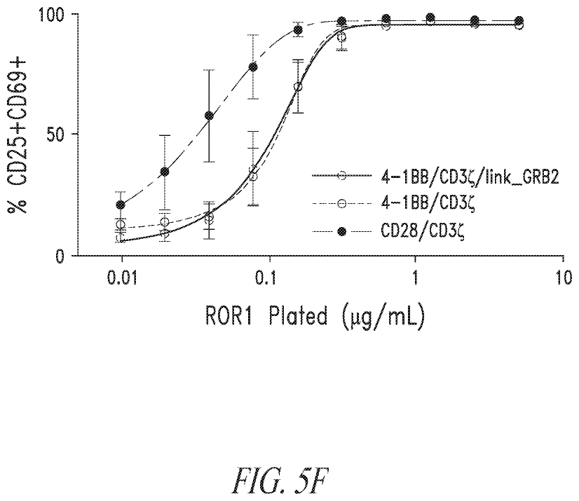

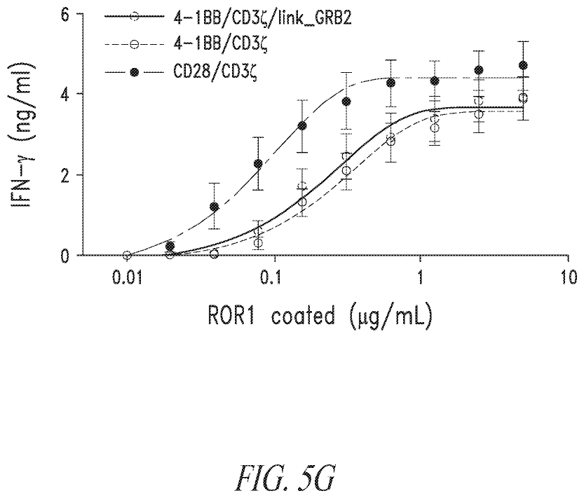

In a further effort to improve LAT PO₄, CARs bearing a GRB2 or GRAP2 SH2 domain after CD3ζ with or without a flexible linker separating the CD3ζ and SH2 domains were constructed (FIG. 4F) (see Whitlow et al., Protein Eng. 6:989-995 (1993)). The CAR with a GRB2 SH2 domain connected by a flexible linker to CD3ζ was expressed in whole cell lysates and on the cell surface although its surface expression was approximately two-fold lower than that of the 4-1BB/CD3ζ CAR lacking GRB2 SH2 (FIGS. 4G-4I). Co-culture of the T cells expressing the most highly expressed 4-1BB/CD3ζ/link_GRB2 CAR with K562/ROR1, MDA-MB-231, and parental K562 tumor cells confirmed that the 4-1BB/CD3ζ/link_GRB2 CAR was functional and secreted similar quantities of IFN-γ, IL-2, and TNF-α to conventional 4-1BB/CD3ζ CAR T cells in an antigen-dependent manner (FIGS. 4J-4L). Having designed functional CARs incorporating GRB2 sequences, fluorescence microscopy measurements of Ca²⁺ mobilization were used to compare the antigen sensitivity of the GRB2 CAR to non-GRB2-containing 4-1BB/CD3ζ CARs. Clear differences were observed between CAR constructs in the kinetics and fraction of activated CAR T cells within minutes of antigen encounter. Even at a 10-fold higher antigen density than was used in FIG. 1C, fewer than 10% of 4-1BB/CD3ζ CAR T cells were activated during 20 minutes of exposure to a ROR1-containing bilayer. In contrast, significantly greater fractions of 4-1BB/CD3ζ/link_GRB2 CAR T cells were more rapidly triggered (FIGS. 5A and 5B). The fraction of responding 4-1BB/CD3ζ/link_GRB2 CAR T cells was higher than that of 4-1BB/CD3ζ CAR T cells, despite 2-fold lower expression of the GRB2-containing CAR on T cells. Notably, the enhanced Ca²⁺ mobilization responses of 4-1BB/CD3ζ/link_GRB2 CAR T cells compared to 4-1BB/CD3ζ persisted through a range of ROR1 densities (FIG. 5C). To investigate whether the GRB2 SH2 domain also augmented proximal phosphoprotein signaling, Western blot was used to measure PO₄ of signaling molecules after 10 minutes of ROR1 bead stimulation. 4-1BB/CD3ζ/link_GRB2 CAR stimulation promoted marginally increased PO₄ of LAT Tyr¹⁹¹, SLP-76 Ser³⁷⁶, or PLC-γ1 Tyr⁷⁸³ PO₄ relative to 4-1BB/CD3ζ CAR T cells, but not CD28/CD3ζ CAR T cells (FIGS. 5D and 5E).

To determine whether Ca²⁺ mobilization and improved phosphorylation of key signaling molecules correlated with downstream activation and effector responses, CD25 and CD69 upregulation as well as IFN-γ production were measured in response to titrated quantities of antigen. 4-1BB/CD3ζ/link_GRB2 CAR T cells exhibited nearly equivalent CD25 and CD69 upregulation, but more IFN-γ production, after exposure to limiting amounts of cognate antigen (FIGS. 5F and 5G). Taken together, these results show that the addition of GRB2 SH2 sequence to a 4-1BB/CD3ζ CAR backbone enhanced intracellular signaling and antigen sensitivity without markedly increasing pro-inflammatory cytokine production, which has been associated with a CD28 costimulatory domain and can lead to severe cytokine release syndrome (see Maloney, Nat. Rev. Clin. Oncol. 2018 15:4 16 279-280 (2019)).

### Example 5

### Inclusion of a SH2 Domain Enhances Car Antitumor Function in Antigen^{Low} Settings and does not Interfere with Function in Antigen^{High} Settings

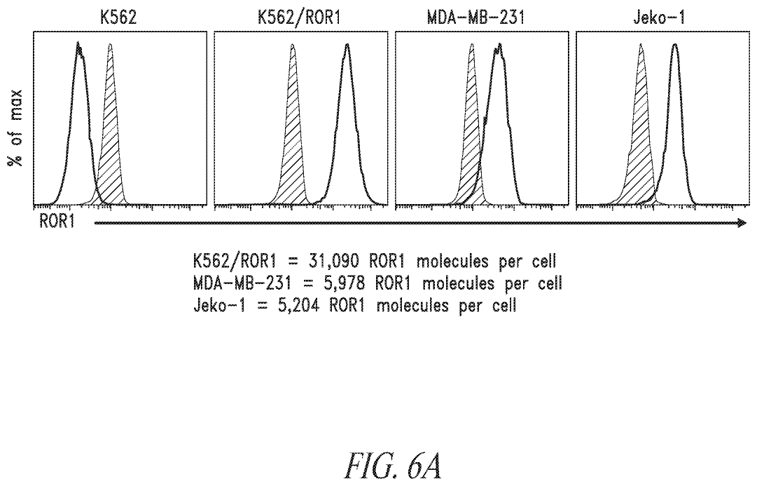

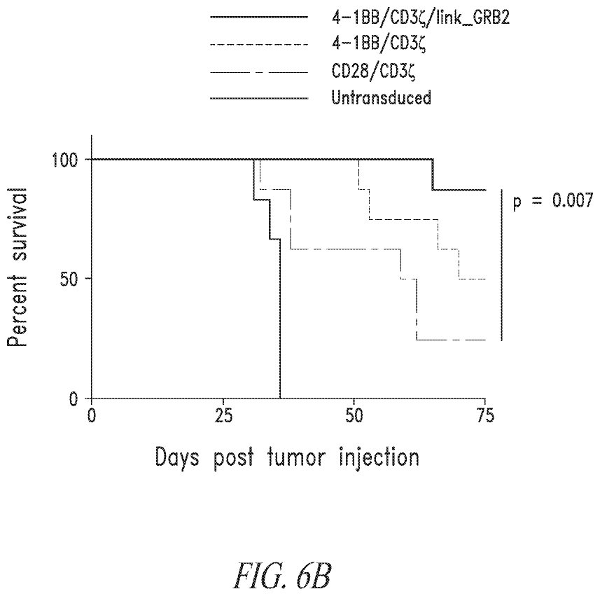

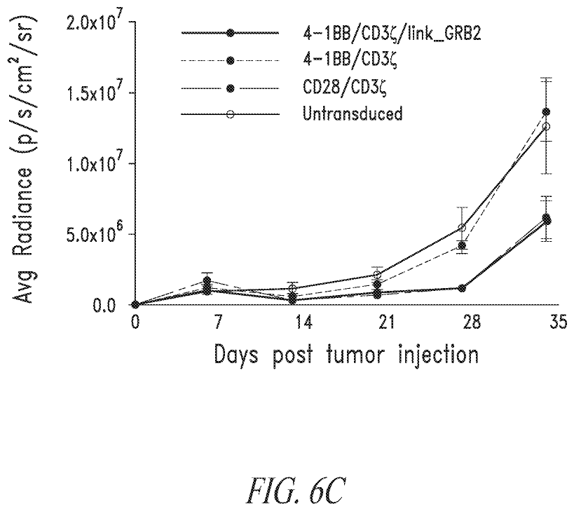

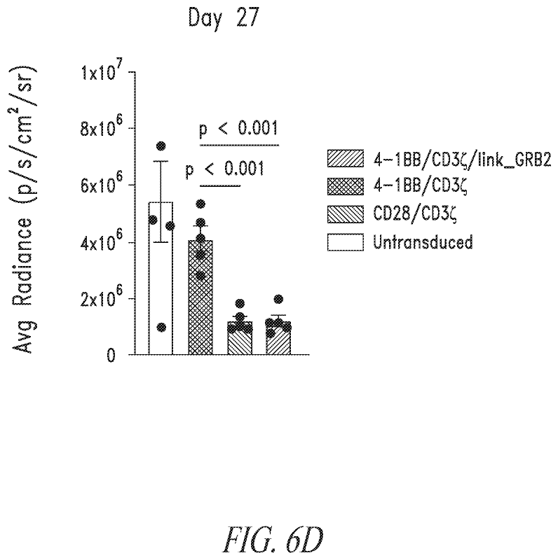

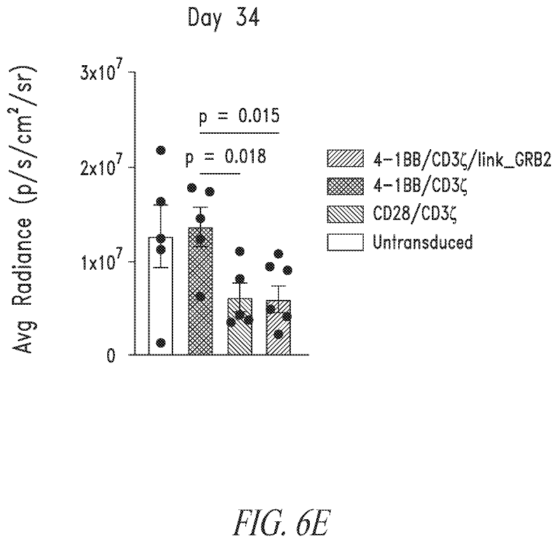

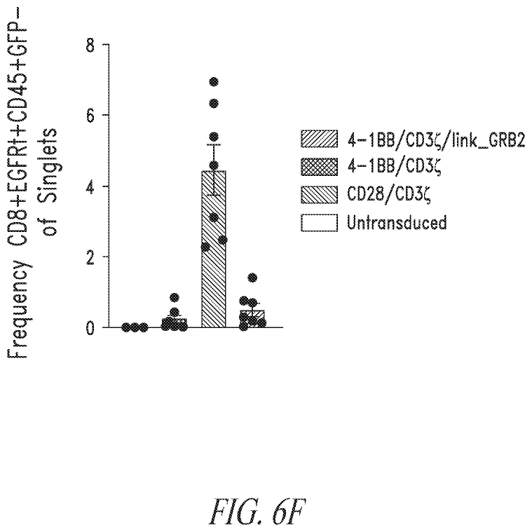

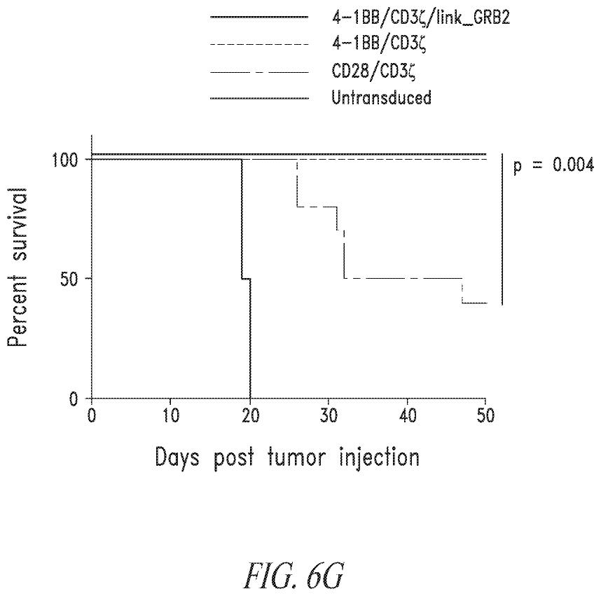

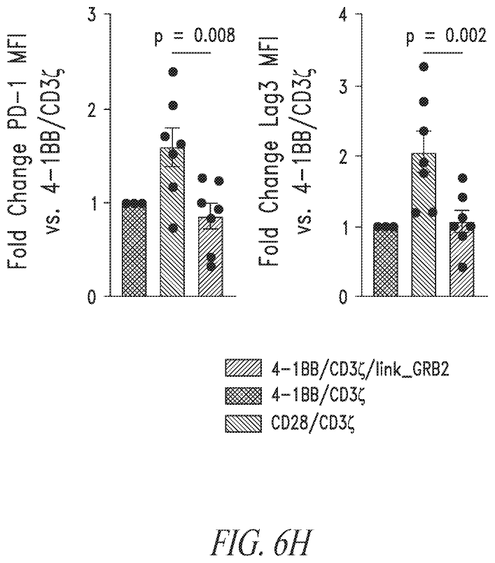

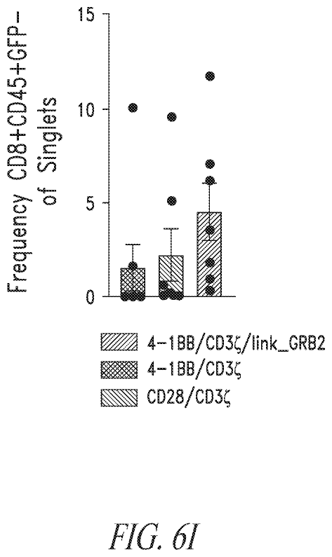

To evaluate whether the improved antigen sensitivity of 4-1BB/CD3ζ/link_GRB2 CAR design translated into improved tumor control in vivo, the phenotype and antitumor activity of the CAR T cells in xenograft mouse models was studied. A disseminated model of Jeko-1 mantle cell lymphoma with low levels of ROR1 expression (5,204 molecules per cell) was first used (FIG. 6A). Infusion of a sub-curative dose of 4-1BB/CD3ζ/link_GRB2 CAR T cells prolonged survival compared to 4-1BB/CD3ζ and CD28/CD3ζ CAR T cells (FIG. 6B). It was notable that CD28/CD3ζ CAR T cells, which possessed the highest antigen sensitivity and strongest intracellular signaling but contained a CD28 costimulatory domain, were least effective at controlling tumor growth.

The activity of the GRB2 SH2-containing CAR T cells and non-GRB2-SH2 comparator CAR T cells in a solid tumor xenograft model of MDA-MB-231 breast adenocarcinoma with low ROR1 expression (˜5,978 molecules per cell; FIG. 6A) was then compared. Seven days after subcutaneous tumor engraftment into the right flank, mice were treated intravenously with a sub-curative dose of CAR T cells. 4-1BB/CD3ζ CAR T cells did not generate a robust antitumor effect, despite their ability to recognize MDA-MB-231 cells in a simple in vitro co-culture (FIGS. 6C and 4K). In contrast, 4-1BB/CD3ζ/link_GRB2, and CD28/CD3ζ CAR T cells demonstrated antitumor effects that were comparable and superior to 4-1BB/CD3ζ CAR T cells, especially at late time points. On experimental days 27 and 34, mice treated with 4-1BB/CD3ζ/link_GRB2 or CD28/CD3ζ CAR T cells had significantly lower tumor burden than mice treated with 4-1BB/CD3ζ or untransduced (UT) T cells (FIGS. 6D and 6E). The improved tumor control of 4-1BB/CD3ζ/link_GRB2 versus 4-1BB/CD3ζ occurred with similar CAR T cell frequencies in the tumor 13 days after T cell transfer (FIG. 6F). Together with data from the Jeko-1 model, these results suggest that enhancing the antigen sensitivity of 4-1BB costimulatory-domain based CARs yields improved in vivo efficacy in settings with low ROR1 expression.

The intense signal strength mediated by CD28/CD3ζ CARs is linked to the development of T cell exhaustion in settings of high antigen density (see Salter et al. (supra); Cherkassky et al., J. Clin. Invest. 126:3130-3144 (2016); Feucht et al., Nat. Med. 545:423 (2018). Sequelae of stronger GRB2 CAR signaling in a xenograft model where tumor cells express high antigen levels were therefore assessed using a model of disseminated CD19⁺ Raji lymphoma that expresses ˜14,872 CD19 molecules per cell. CD19-specific CARs with the GRB2 SH2 domain were also created. Infusion of a low sub-curative dose of CD28/CD3ζ CAR T cells modestly improved survival compared to a mock injection of saline (FIG. 6G).

However, an equivalent dose of T cells expressing the novel 4-1BB/CD3ζ/link_GRB2 CARs, or a conventional 4-1BB/CD3ζ CAR, promoted marked improvements in survival. Additional analyses of CAR T cells in the bone marrow 14 days after T cell transfer showed that 4-1BB/CD3ζ/link_GRB2 CAR T cells expressed lower amounts of the exhaustion-associated inhibitory molecules PD-1 and LAG-3 than CD28/CD3ζ CAR T cells and were present at a higher frequency (FIGS. 6H and 6I). The pattern of PD-1 and LAG-3 expression mirrored antigen sensitivity measurements and showed that higher levels of inhibitory receptor expression correlated with reduced tumor control. Thus, unlike the highly antigen sensitive CD28/CD3ζ CAR that also promotes T cell dysfunction, the exemplary SH2-containing CARs retained potent antitumor activity against CD19⁺ and ROR1⁺ tumor cells that express high and low levels of antigen respectively.

### Example 6

### Design and Testing of a CAR Containing a GRB2 Superbinder Sequence

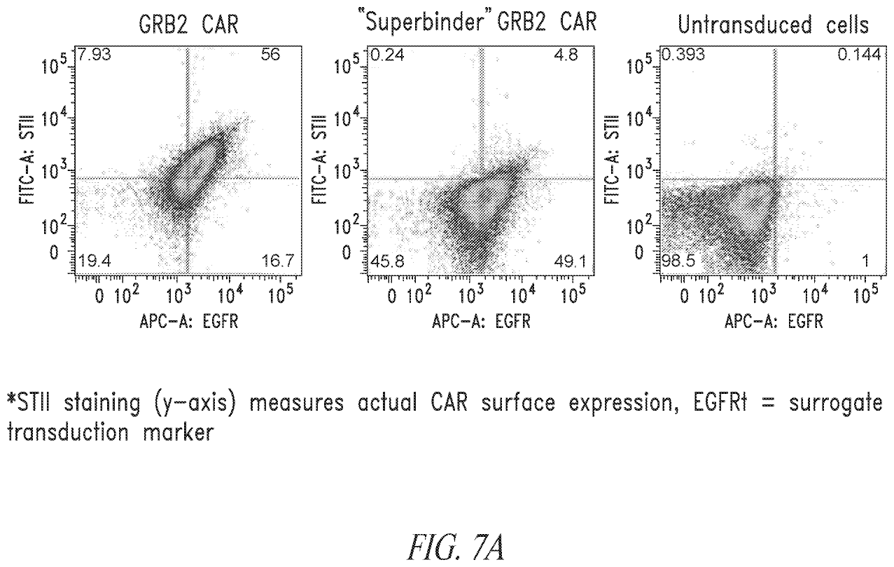

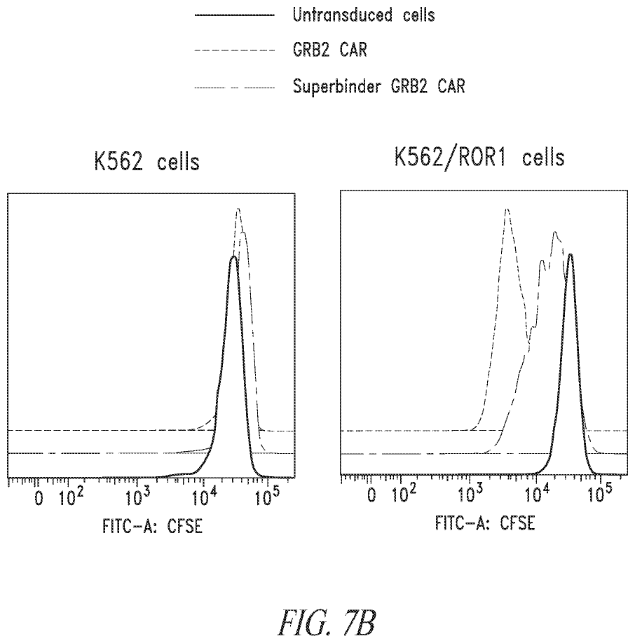

A modified GRB2 SH2 domain engineered for enhanced affinity for proteins with phosphorylated tyrosine residues has been reported (referred to as a “superbinder” SH2 domain; Kaneko et al., Science Signaling 5:243 ra68 (2012)). Because TCR signaling utilizes phosphorylated tyrosine residues to transmit cellular messages, a 4-1BB/CD3ζ/link_GRB2 CAR architecture was generated that contained a GRB2 superbinder domain and was tested for enhanced CAR T cell efficacy compared to the CAR containing an unmodified GRB2 SH2 sequence. The superbinder GRB2 CAR was expressed on the cell surface of purified CD8⁺EGFRt⁺ T cells, albeit at lower levels than the CAR containing wild type GRB2 (FIG. 7A). Additionally, CAR T cells with the Superbinder GRB2 domain proliferated less robustly than unmodified GRB2 CAR T cells, after co-culture with K562/ROR1 tumor cells (FIG. 7B).

### Example 7

### Materials and Methods

### Cell Culture

LentiX cells (Clontech) were cultured in DMEM (Gibco) supplemented with 10% fetal bovine serum, 1 mM L-glutamine (Gibco), 25 mM HEPES (Gibco), and 100 U/mL penicillin/streptomycin (Gibco). Jeko-1 (CRL-3006), K562 (CCL-243), MDA-MB-231 (HTB-26), NCI-H358 (CRL-5807), and Raji (CCL-86) cells were obtained from American Type Culture Collection and cultured in RPMI-1640 (Gibco) supplemented with 5% fetal bovine serum, 1 mM L-glutamine, 25 mM HEPES, and 100 U/mL penicillin/streptomycin. Primary human T cells were cultured in CTL medium consisting of RPMI-1640 supplemented with 10% human serum, 2 mM L-glutamine, 25 mM HEPES, 100 U/mL penicillin/streptomycin and 50 μM β-mercaptoethanol (Sigma). All cells were cultured at 37° C. and 5% CO₂, and tested bi-monthly for the absence of mycoplasma using MycoAlert Mycoplasma Detection Kit (Lonza).

### Generation of Transduced Tumor Cell Lines

K562 cells expressing HLA-B8 were derived by transduction with lentivirus supernatant prepared from LentiX cells that were transiently transfected with psPAX2, pMD2.G, and a lentiviral vector encoding HLA-B8. psPAX2 and pMD2.G were gifts from Didier Trono (Addgene plasmid #'s 12259 and 12260). K562/B8 cells expressing EBV antigens were derived by transduction with retrovirus supernatant prepared from LentiX cells that were transiently transfected with MLV g/p, 10A1, and a retroviral vector encoding GFP and an EBV peptide minigene. K562 and K562/B8 cells expressing human ROR1 were derived by transduction with lentivirus supernatant prepared from LentiX cells that were transiently transfected with MLV g/p, 10A1, and a retroviral vector encoding human ROR1 (UniProt Q01973, aa1-973). Jeko-1/ffluc, MDA-MB-231/ffluc, and Raji/ffluc cells were derived by transduction with lentivirus supernatant prepared from LentiX cells that were transiently transfected with psPAX2, pMD2.G, and a lentiviral vector encoding GFP and firefly luciferase. For all transductions, viral supernatant was harvested 48 hours after transfection of LentiX cells, filtered using a 0.45 μm PES syringe filter (Millipore), and added to tumor cells in the presence of Polybrene (Millipore) at a final concentration of 4.4 μg/mL. Five days later, transduced tumor cells were enriched on a FACSAria II (BD Biosciences) to greater than 97% purity.

### Generation of Chimeric Antigen Receptor (CAR) Lentiviral Vectors

Previously described ROR1-specific 4-1BB/CD3ζ and CD28/CD3ζ CAR constructs with an R12 scFv, modified IgG4 hinge and CD28 transmembrane domain were used for LC-MS/MS signaling analyses in bi-specific T cells (Hudecek et al., Clin. Cancer Res. 19:3153-3164 (2013); Lamers et al., J. Clin. Oncol. 24:e20-2 (2006)). CAR design changes were carried out using a ROR1-specific 4-1BB/CD3ζ backbone containing a StrepTag II (STII) sequence and two G4S linkers inserted between the R12 scFv and immunoglobulin G4 (IgG4) hinge. LAT_{TMD} CARs were developed by swapping in two versions of the LAT transmembrane domain to the CAR backbone in place of the CD28 transmembrane domain. The HCH transmembrane domain utilized amino acids 5-30 of human LAT (UniProt: 043561); the EXT transmembrane domain utilized amino acids 1-35 of human LAT (UniProt: 043561). GRAP2 and GRB2 CARs were constructed by adding an 18-amino acid Whitlow linker (linker 218 (SEQ ID NO:63) followed by the GRAP2 SH2 domain (UniProt: 075791, aa58-149) (SEQ ID NO:9) or GRB2 SH2 domain (UniProt: P62993, aa60-152) (SEQ ID NO:7). CAR constructs were codon-optimized and linked by T2A sequence to truncated CD19 (CD19t) or epidermal growth factor receptor (EGFRt), and cloned into a HIV7 lentiviral vector. All cloning was performed by PCR, enzyme digest, and/or Gibson assembly. Plasmids were verified by capillary sequencing. Lentivirus was generated by transient transfection of LentiX cells using psPAX2, pMD2.G, and the CAR-encoding lentiviral vector.

Acquisition of Peripheral Blood T Cells from Healthy Donors

Healthy adults (>18 years-old) were enrolled in Institutional Review Board-approved studies for peripheral blood collection. Informed consent was obtained from all enrollees. Researchers were provided donor age, nondescript donor ID number, human leukocyte antigen haplotype, and Epstein-Barr virus serology results, and were blinded to all other personally identifiable information about study participants. PBMC were isolated by density gradient using Lymphocyte Separation Media (Corning). CD8⁺ and memory CD8⁺ T cells were further isolated using the EasySep Human CD8⁺ and Memory CD8⁺ T Cell Isolation/Enrichment Kits (StemCell Technologies) in accordance with manufacturer's instructions.

### T Cell Transduction and Culture

To prepare bi-specific T cells, memory CD8⁺ T cells were stimulated using irradiated autologous PBMC that had been pulsed with EBV-RAK peptide (RAKFKQLL, Elim Biopharmaceuticals) in AIM V media (Gibco). T cells and PBMC were cultured at a 1:1 ratio in CTL medium supplemented with 50 IU/mL human IL-2 (Prometheus). Two days later, lentiviral supernatant that encoded a ROR1 CD28/CD3ζ or 4-1BB/CD3ζ CAR and CD19t transduction marker was harvested from LentiX cells and added to the T cell and PBMC co-culture. Polybrene (Millipore) was added at a final concentration of 4.4 μg/mL and T cells were spinoculated at 800 g and 32° C. for 90 minutes. Viral supernatant was replaced 8 hours later with fresh CTL supplemented with 50 IU/mL IL-2. Half-media changes were then performed every 48 hours using CTL supplemented with 50 IU/mL IL-2. Transduced CD8⁺ tetramer⁺ CD19t⁺ T cells were sorted on a FACSAria II on day 11.

To prepare conventional CAR T cells with polyclonal TCR specificities, CD8⁺ T cells were activated using Dynabeads Human T-Activator CD3/CD28 (ThermoFisher) at a 3:1 bead to T cell ratio and cultured in CTL medium supplemented with 50 U/mL IL-2. The next day, lentiviral CAR-encoding lentiviral supernatant was added to the activated T cells. Polybrene was added at a final concentration of 4.4 μg/mL and the T cells were spinoculated at 800 g and 32° C. for 90 minutes. Viral supernatant was replaced 8 hours later with fresh CTL medium supplemented with 50 IU/mL IL-2. Half-media changes were then performed every 48 hours using CTL supplemented with 50 IU/mL IL-2. Dynabeads were removed on day 5, CD8⁺EGFRt⁺ transduced T cells were FACS-sorted on day 8 to obtain highly enriched CAR T cells. Enriched CAR T cells were either immediately expanded if large numbers were necessary for experimentation (detailed in subsequent section), or cultured in CTL supplemented with 50 IU/mL IL-2 until day 12-14. On days 12-14, T cells were used for functional assays or injected into mice.

### T Cell Expansion for Signaling CAR Expression, and Ca²⁺ Flux Analyses

FACS-purified CD8⁺ tetramer⁺ CD19t⁺ or CD8⁺EGFRt⁺ cells were expanded using 30 ng/mL purified OKT3, γ-irradiated LCL (8,000 rad), and γ-irradiated (3,500 rad) allogeneic PBMC at a LCL to T cell ratio of 100:1 and a PBMC to T cell ratio of 600:1. 50 IU/mL IL-2 was added on day 1, OKT3 was washed out on day 4, cultures were fed with fresh CTL medium supplemented with 50 IU/mL IL-2 every 2-3 days and resting T cells were used for assays 11-12 days after stimulation.

### Flow Cytometry and Cell Phenotyping

T cells were stained with a 1:100 dilution of fluorophore-conjugated monoclonal antibodies specific for human CD4 (RPA-T4), CD8 (SK1), CD19 (HIB19), CD28 (CD28.2), CD45 (HI30), CD45RO (UCHL1), CD62L (DREG56), CD223 (3DS223H), CD279 (eBioJ105) or EGFR (AY13) purchased from BD Biosciences, ThermoFisher, or Biolegend. Phycoerythrin (PE)-conjugated HLA-B8/EBV tetramer was generated by the Immune Monitoring Core Facility at the Fred Hutchinson Cancer Research Center. T cells were also stained with isotype control fluorophore-conjugated antibodies when appropriate. Biotinylated Cetuximab (anti-EGFR, Bristol Myers Squibb) was prepared and used in conjunction with Streptavidin-APC (ThermoFisher) as previously described (Salter et al. (supra)). DNA content staining was performed by fixing T cells with 70% ice-cold ethanol, permeabilizing cells with 1% Triton-X (Sigma), degrading RNA with 100 μg/mL RNAse A (ThermoFisher), and staining DNA with 20 μg/mL Propidium Iodide (ThermoFisher). All data was collected on a FACSCanto II, FACSCelesta, or FACSAria II (BD Biosciences). FlowJ0 version 9 (Treestar) was used to analyze flow cytometry files.

### SCT, SCT/CD28, ROR1, STII and Control Microbead Preparation

Recombinant single chain trimer (SCT) and human ROR1 containing a C-terminal Avi tag were produced by the Molecular Design and Therapeutic program at the Fred Hutchinson Cancer Research Center. SCT and ROR1 were biotinylated using BirA Ligase (Avidity) and desalted using PD-10 columns (GE Healthcare). 1 mL Streptavidin Coated Magnetic Particles (Spherotech) was washed once in excess 1×PBS supplemented with 100 U/mL penicillin/streptomycin (PBS+P/S) using a benchtop magnet. SCT and ROR1 microbeads were prepared by resuspending beads in 1 mL PBS+P/S and then slowly adding biotinylated recombinant protein while vortexing the solution. Protein was added at the indicated molar ratios according to manufacturer's predetermined molar binding capacities. SCT/CD28 microbeads were prepared by resuspending beads in 1 mL PBS+P/S and then slowly adding recombinant SCT protein and biotinylated CD28 mAb (CD28.2, ThermoFisher) at a 3:1 molar ratio. All microbeads were incubated overnight at 4° C. on a 3D orbital shaker, washed three times with excess PBS+P/S using a benchtop magnet, and resuspended in 1 mL PBS+P/S. To make control beads, 1 mL Streptavidin Coated Magnetic Particles was washed once using a benchtop magnet and the bead pellet was resuspended in 1 mL PBS+P/S. All beads were stored at 4° C.

### In Vitro Functional Assays

CAR T cells were co-cultured with γ-irradiated (10,000 rad) K562, K562/B8, K562/ROR1, or MDA-MB-231 cells at a T cell to tumor cell ratio of 2:1. In some experiments, CAR T cells were also incubated with control, SCT or SCT/CD28 microbeads at a ratio of 7.5 mL beads per million cells. For antigen sensitivity experiments, T cells were incubated on plates that had previously been coated with varying quantities of ROR1 recombinant protein. Cytokine concentrations in cellular supernatant were quantified by ELISA (ThermoFisher) 24 hours after stimulation. T cell proliferation was quantified by staining T cells with a 0.2 μM solution of carboxyfluorescein succinimidyl ester (CFSE) dye (ThermoFisher) prior to incubation with microbeads for 72 hours.

### Fluorescence Microscopy

CD8⁺ T cells were transduced as previously described. Instead of FACS purification on day 9, cells were imaged on a DeltaVision Elite microscope (GE Healthcare). At least ten cells were visualized per condition. Raw images were subjected to a linear adjustment of brightness and contrast using ImageJ (NIH).

### Ca²⁺ Mobilization Measurements

Ca²⁺ mobilization was measured using flourecence microscopy of T cells loaded with the Ca²⁺-sensitive dye Flou-4 AM. Ca²⁺ flux was monitored in hundreds of individual cells simultaneously as they came into contact with supported lipid bilayers functionalized with ROR1 via biotin/streptavidin linkage. Ligand-functionalized bilayers were prepared within imaging flow chambers. Briefly, bilayers were formed via deposition of large unilamellar vesicles (LUVs) onto NoChromix (Godax laboratories)-cleaned glass within imaging flow cells. Lipid mixtures contained mole fractions ranging from 0.005% to 0.5% of 16:0 biotinyl cap PE in Egg PC (Avanti). Bilayers were fluorescently labeled and functionalized with ligand through successive incubation with Atto655-streptavidin (Sigma Aldrich) followed by biotinylated SCT or ROR1.

For each imaging experiment, 2-5×10⁵ CAR T cells were loaded with the calcium-sensitive dye Fluo-4 AM (Thermo Fisher). Cells were loaded for 2 min at 37° C. with 2.5 mg/mL Fluo-4 AM in HEPES-buffered saline (HBS) containing 1 mg/mL BSA and 0.25 mM sulfinpyrazone. The cell suspension was then diluted to a final volume of 8 mL with HBS/BSA/sulfinpyrazone and incubated at 37° C. for 30 min. Cells were pelleted and resuspended twice to remove excess dye and resuspended a final time in 100 mL HBS/BSA/sulfinpyrazone. Imaging experiments were conducted on an Olympus IX81-XDC inverted microscope using an Andor iXon-987 EMCCD and epi-fluorescence illumination at 488 nm with a CoolLED pE light source. Cells were imaged at room temperature and 10× magnification at 2 frames per second for 20 min, beginning immediately after dye-loaded cells were added to the flow chamber. Imaging data was processed in MATLAB (Mathworks) using custom data analysis routines. Cells were localized using a watershed segmentation algorithm and pixel intensities within cell boundaries were quantified. Cell positions were tracked to monitor intensity signatures of individual cells over the course of the experiment. Cellular activation times were defined as the first time point where Flou-4 intensity surpasses three times the baseline fluorescence level of each cell before Ca²⁺ mobilization. Cumulative plots of single cell activation times were fit to exponential functions to extract time constants and total fraction of activated cells for the population response.

### TCR or CAR Stimulation and Protein Lysate Generation

T cells were washed and resuspended in warm CTL medium. T cells were incubated with control, SCT, SCT/CD28 or ROR1 microbeads in a 37° C. water bath at a final cell concentration of 2×10⁷ cells per mL and a bead to cell ratio of 7.5 μL per million cells, unless otherwise indicated. In some experiments, bi-specific T cells were incubated with K562/B8, K562/B8/CE, and K562/B8/ROR1 tumor cells at a T cell to tumor cell ratio of 4:1. After the allotted time, cells were quickly washed twice using ice-cold PBS, then lysed in a 6M Urea, 25 mM Tris (pH 8.0), 1 mM EDTA, 1 mM EGTA solution supplemented with protease (Sigma) and phosphatase inhibitors (Sigma) at a 1:100 dilution, hereon referred to as lysis buffer. Lysates were sonicated for 15 seconds prior to centrifuging at 10,000 g and 4° C. for 10 minutes. Beads were removed during lysate clearing and protein concentration was quantified by BCA Assay or Micro BCA Assay (ThermoFisher).

### Western Blotting

Equal masses of protein lysate were loaded into 4-12% Bis-Tris NuPAGE Gels (ThermoFisher). After protein transfer onto 0.45 μm nitrocellulose membranes (ThermoFisher), membranes were blocked with Western Blocking Reagent (Sigma) diluted 1:10 in 1×Tris-buffered saline (TBS). Membranes were stained with primary and secondary antibodies diluted 1:5,000-1:10,000 in SuperBlock TBS (ThermoFisher) supplemented with 0.1% Tween. The following antibodies were used: anti-CD247 (8D3, BD Biosciences), anti-CD247 pTyr¹⁴² (K25-407.69, BD Biosciences), anti-LAT (polyclonal, Cell Signaling), anti-LAT pTyr¹⁹¹ (polyclonal, Cell Signaling), anti-PLC-g1 (D9H10, Cell Signaling), anti-PLC-g1 pTyr⁷⁸³ (D6M9S, Cell Signaling), anti-SLP-76 (polyclonal, Cell Signaling), anti-SLP-76 pSer³⁷⁶ (D9D6E, Cell Signaling), anti-ZAP-70 (D1C10E, Cell Signaling), anti-ZAP-70 pTyr³¹⁹ (65E4, Cell Signaling), anti-mouse horseradish peroxidase (HRP) (polyclonal, Cell Signaling), and anti-rabbit HRP (polyclonal, Cell Signaling). Band intensities were quantified using ImageJ [National Institutes of Health (NIH)]; normalized to total protein or loading control, and then renormalized to a control sample.

Protein Digestion, TMT Labeling and Phosphotyrosine (pTyr) Peptide Immunoprecipitation

Lysates were diluted to 2 mg/mL using lysis buffer. Lysates were reduced in 24 mM TCEP (ThermoFisher) for 30 minutes at 37° C. with shaking, followed by alkylation with 48 mM iodoacetamide (Sigma) in the dark at room temperature for 30 minutes. Lysates were then diluted with 200 mM Tris (pH 8.0), to a urea concentration of 2M. Lys-C(Wako) was dissolved in 25 mM Tris (pH 8.0) at 200 ug/mL and added to lysates at 1:100 (enzyme:protein) ratio by mass and incubated for 2 hours at 37° C. with shaking. Samples were further diluted with 200 mM Tris (pH 8.0) to a urea concentration of 1M before adding trypsin at a 1:50 trypsin:protein ratio. After 2 hours, a second trypsin aliquot was added at a 1:100 trypsin:protein ratio. Digestion was carried out overnight at 37° C. with shaking. After 16 hours, the reaction was quenched with formic acid to a final concentration 1% by volume. Samples were desalted using Oasis HLB 96-well plates (Waters) and a positive pressure manifold (Waters). The plate wells were washed with 3×400 μL of 50% MeCN/0.1% FA, and then equilibrated with 4×400 μL of 0.1% FA. The digests were applied to the wells, then washed with 4×400 μL 0.1% FA before being eluted drop by drop with 3×400 μL of 50% MeCN/0.1% FA. The eluates were lyophilized, followed by storage at −80° C. until use. For TMT labeling (ThermoFisher), desalted peptides were resuspended in 50 mM HEPES at 1 mg/mL based on starting protein mass. TMT reagents were resuspended in 257 μL MeCN and transferred to the peptide sample. Samples were incubated at room temperature for 1 hour with mixing. Labeling reactions were quenched by the addition of 50 μL of 5% hydroxyl Amine (Sigma) and incubated for 15 minutes at room temperature with mixing. The independent labeling reactions were then pooled together and lyophilized. The labeled peptides were desalted as above and then lyophilized and stored at −80° C. Immunoprecipitation of pTyr peptides was performed using the PTMScan P-Tyr-1000 Kit (Cell Signaling). The enriched pTyr peptide fraction was purified using a C18 Spin Tip (ThermoFisher), lyophilized, and stored at −80° C. until analysis. The flow-through fraction was desalted, lyophilized, and stored at −80° C.

### Basic (High pH) Reverse Phase Liquid Chromatography

The desalted and pTyr peptide-depleted flow-through was fractionated by high-pH reverse phase (RP) liquid chromatography. 4 mg of the protein digest was loaded onto a LC system consisting of an Agilent 1200 HPLC with mobile phases of 5 mM NH₄HCO₃ (pH 10) (A) and 5 mM NH₄HCO₃ in 90% MeCN (pH 10) (B). The peptides were separated by a 4.6 mm×250 mm Zorbax Extend-C18, 3.5 μm, column (Agilent) over 96 minutes at a flow rate of 1.0 mL/min by the following timetable: hold 0% B for 9 minutes, gradient from 0 to 10% B for 4 minutes, 10 to 28.5% B for 50 minutes, 28.5 to 34% B for 5.5 minutes, 34 to 60% B for 13 minutes, hold at 60% B for 8.5 minutes, 60 to 0% B for 1 minute, re-equilibrate at 0% B for 5 minutes. 1 minute fractions were collected from 0-96 minutes by the shortest path by row in a 1 mL deep well plate (ThermoFisher). The high pH RP fractions were concatenated into 24 samples by every other plate column starting at minute 15 (e.g.: sample 1 contained fractions from wells B10, D10, F10, etc.). The remaining fractions were combined such that fractions from 12 to 14 minutes were added to sample 1, all fractions after 86 minutes were added to sample 24, and all fractions from 0 to 11 minutes were combined into sample ‘A’. 95% of every 12th fraction of the 24 samples was combined (1,13; 2,14; . . . ) to generate 12 more samples, which were dried down and stored at −80° C. prior to phosphopeptide enrichment by immobilized metal affinity chromatography.

### Immobilized Metal Affinity Chromatography (IMAC)

IMAC enrichment was performed using Ni-NTA-agarose beads (Qiagen) stripped with EDTA and incubated in a 10 mM FeCl₃ solution to prepare Fe³⁺-NTA-agarose beads. Fractionated lysate was reconstituted in 200 μL of 0.1% TFA in 80% MeCN and incubated for 30 minutes with 100 μL of the 5% bead suspension while mixing at room temperature. After incubation, beads were washed 3 times with 30011L of 0.1% TFA in 80% MeCN. Phosphorylated peptides were eluted from the beads using 200 μL of 70% ACN, 1% Ammonium Hydroxide for 1 minute with agitation at room temperature. Samples were transferred into a fresh tube containing 60 μL of 10% FA, dried down and re-suspended in 0.1% FA, 3% MeCN. Samples were frozen at −80° C. until analysis.

### Nano-Liquid Chromatography-Tandem Mass Spectrometry

Phosphopeptide-enriched samples were analyzed by LC-MS/MS on an Easy-nLC 1000 (ThermoFisher) coupled to an LTQ-Orbitrap Fusion mass spectrometer (ThermoFisher) operated in positive ion mode. The LC system, configured in a vented format consisted of a fused-silica nanospray needle (PicoTip emitter, 50 μm ID×20 cm, New Objective) packed in-house with ReproSil-Pur C18-AQ, 3 μm and a trap (IntegraFrit Capillary, 100 μm ID×2 cm, New Objective) containing the same resin as in the analytical column with mobile phases of 0.1% FA in water (A) and 0.1% FA in MeCN (B). The peptide sample was diluted in 20 μL of 0.1% FA, 3% MeCN, and 8.5 μL was loaded onto the column and separated over 210 minutes at a flow rate of 300 nL/min with a gradient from 5 to 7% B for 2 minutes, 7 to 35% B for 150 minutes, 35 to 50% B for 1 minute, hold 50% B for 9 minutes, 50 to 95% B for 2 minutes, hold 95% B for 7 minutes, 95 to 5% B for 1 minute, re-equilibrate at 5% B for 38 minutes. A spray voltage of 2000 V was applied to the nanospray tip. MS/MS analysis occurred over a 3 second cycle time consisting of 1 full scan MS from 350-1500 m/z at resolution 120,000 followed by data dependent MS/MS scans using HCD activation with 27% normalized collision energy of the most abundant ions. Selected ions were dynamically excluded for 45 seconds after a repeat count of 1.

Transfer of T cells in NOD SCID/γc^{-/-} (NSG) mice

Six- to eight-week-old male or female NSG mice were obtained from the Jackson Laboratory or bred in-house. Mice were engrafted with 5×10⁵ MDA-MB-231/ffluc subcutaneously in the right flank, or with intravenously with Jeko/ffluc or Raji/ffluc cells. Seven days later, mice were injected intravenously with purified CD8⁺ CAR T cells, mock transduced CD8⁺ T cells, or saline. Mice were followed for survival or sacrificed at various time points. For T cell phenotypic analysis, tumor or bone marrow was harvested. Single cell suspensions from tumor were prepared using Mouse Tumor Dissociation Kit (Miltenyi Biotec); suspensions from bone marrow were prepared by crushing using a mortar and pestle, filtering using a 0.7 μm filter, and lysing red blood cells using ACK Lysing Buffer (Gibco). Resulting single cell suspensions were stained with fluorochrome-conjugated monoclonal antibodies for flow cytometric analysis. Mice handlers were blinded to group allocation. The Fred Hutchinson Cancer Research Center Institutional Animal Care and Use Committee approved all experimental procedures.

### Bioluminescence Imaging of Tumor Growth

Mice received intraperitoneal injections of luciferin substrate (Caliper Life Sciences) resuspended in 1×PBS (15 mg per g body weight). Mice were anesthetized with isoflurane and imaged using an Xenogen IVIS Imaging System (Caliper Life Sciences) 10, 12 and 13 min after luciferin injection in small binning mode at an acquisition time of 15 sec to 1 min to obtain unsaturated images. Luciferase activity was analyzed using Living Image Software 4.7.2 (Caliper Life Sciences) and the photon flux analyzed within regions of interest that encompassed the entire body of each individual mouse.

### Shotgun Mass Spectrometry Data Analysis

Raw MS/MS spectra from each replicate experiment were searched together against the reviewed Human Universal Protein Resource (UniProt) sequence database (release 2016_01) with common laboratory contaminants using the MaxQuant/Andromeda search engine version 1.6.0.1 (see Cox & Mann Nat. Biotechnol. 26:1367-1372 (2008)). The search was performed with a tryptic enzyme constraint for up to two missed cleavages. Variable modifications were oxidized methionine, phosphorylated serine, phosphorylated threonine, and phosphorylated tyrosine. Carbamidomethylated cysteine was set as a static modification. Peptide MH+ mass tolerances were set at 20 ppm. The overall FDR was set at <1% using a reverse database target decoy approach.

For the three TMT experiments, phosphopeptide site localization was determined by MaxQuant and converted to phosphorylation sites using Perseus version 1.6.0.7 (see Tyanova et al., Nat. Methods 13:731-740 (2016)). The site table was expanded, reverse hits and potential contaminants were excluded, and any phosphorylation site with fewer than 6 or more values across the nine TMT channels was excluded from further analysis. Data normalization was performed by scaling each TMT channel to the channel median. Stimulation vs. control ratios were calculated by dividing TCR or CAR stimulated channels by the control channel, log 2 transformed, and renormalized by subtracting the column median. Due to incomplete MS sampling, some phosphorylation sites (features) were only found in one or two replicate experiments.

Differential expression analyses over PO₄ sites were performed using the limma statistical framework and associated R package (see Ritchie et al., Nucleic Acids Res. 43:e47 (2015); Smyth, Stat Appl. Gent. Mol. Biol. 3, Article3 (2004)). For these analyses, only those features were kept that had values in at least two experiments and all TMT channels, leaving 19,608 quantified PO₄ sites. A linear model was fitted to each PO₄ site, and empirical Bayes moderated t-statistics were used to assess differences in expression/abundance. Contrasts comparing stimulation vs control treatments were tested. Intraclass correlations were estimated using the duplicateCorrelation function of the limma package to account for measures originating from the same patients and the same antigens (Smyth et al., Bioinformatics 21:2067-2075 (2005)). An absolute log 2 fold change cutoff (stimulation versus control) of 1 and a false discovery rate (FDR) cutoff of 5% were used to determine differentially expressed PO₄ sites. Analyses of signaling networks and KEGG Pathways were performed using StringDB.

### Analysis of T Cell Phenotype, Function, and In Vivo Experiments

Prism version 8 (GraphPad Software) was used to plot data and calculate statistics. P values meeting an α=0.05 level of significance are indicated in the figures. The precise statistical tests used are indicated in the figure legends.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 62/901,186, filed Sep. 16, 2019, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A fusion protein, comprising:
(a) an extracellular component comprising a binding domain that specifically binds to an target;
(b) a transmembrane domain; and
(c) an intracellular component comprising an SH2 domain or a functional portion or variant thereof.

2. The fusion protein of claim 1, wherein the SH2 domain or functional portion or variant thereof is from Grb2, Grap2, Fyn, Src, Grap, CRLK, INPP5D, ITK, LCK, SLP-76, NKC1, NCK2, PIK3R1, PIK3R2, PLCG1, PLCG2, PTPN6, SH2D1A, SHB, Syk, TEC, VAV1, TXK, ZAP70, BLK, BLNK, BMX, BTK, HSH2D, LYN, PTPN11, SH2B2, SH2D1B, SH2D2A, SH2D3C, SH2D4A, SOCS1, STAT1, STAT2, STAT3, STAT4, STAT5A, STAT5B, STAT6, or YES1.

3. The fusion protein of claim 1 or 2, wherein the SH2 domain or functional portion or variant thereof is from, or is derived from, Grb2, Grap2, Fyn, or Src.

4. The fusion protein of any one of claims 1-3, wherein the SH2 domain or functional portion or variant thereof comprises an amino acid sequence having at least about 75% identity to the amino acid sequence shown in any one of SEQ ID NOs.:7-62.

5. The fusion protein of any one of claims 1-4, wherein the SH2 domain or functional portion or variant thereof comprises or consists of the amino acid sequence set forth in SEQ ID NO.:7 or 8.

6. The fusion protein of any one of claims 1-4, wherein the SH2 domain or functional portion or variant thereof comprises or consists of the amino acid sequence set forth in SEQ ID NO.:9.

7. The fusion protein of any one of claims 1-4, wherein SH2 domain or functional portion or variant thereof comprises or consists of the amino acid sequence set forth in any one of SEQ ID NOs.:12-14.

8. The fusion protein of any one of claims 1-4, wherein the SH2 domain or functional portion or variant thereof comprises or consists of the amino acid sequence set forth in SEQ ID NO:10 or 11.

9. The fusion protein of any one of claims 1-8, wherein the intracellular domain further comprises an effector domain, or a functional portion or variant thereof.

10. The fusion protein of claim 9, wherein the effector domain or functional portion or variant thereof is from CD3ζ, CD25, CD79A, CD79B, CARD 11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof.

11. The fusion protein of 9 or 10, wherein the effector domain or functional portion or variant thereof is disposed between the transmembrane domain and the SH2 domain or functional portion or variant thereof.

12. The fusion protein of claim 9 or 10, wherein the SH2 domain or functional portion or variant thereof is disposed between the effector domain or functional portion or variant thereof and the transmembrane domain.

13. The fusion protein of claim 11 or 12, further comprising a linker that is disposed (a) between the SH2 domain or functional portion or variant thereof and the effector domain or functional portion or variant thereof and/or (b) between the SH2 domain or functional portion or variant thereof and the transmembrane domain and/or (c) between the transmembrane domain and the effector domain or functional portion or variant thereof.

14. The fusion protein of claim 13, wherein the linker comprises or consists of an amino acid sequence having at least about 75% identity to the amino acid sequence set forth in any one of SEQ ID NOs.: 63-71.

15. The fusion protein of any one of claims 1-14, wherein the intracellular component further comprises a costimulatory domain or a functional portion or variant thereof.

16. The fusion protein of claim 15, wherein the costimulatory domain or functional portion or variant thereof is from 4-1BB, CD28, OX40, CD27, CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or any combination thereof.

17. The fusion protein of claim 16, wherein the costimulatory domain or functional portion or variant thereof is from 4-1BB.

18. The fusion protein of any one of claims 15-17, wherein the intracellular component comprises:
(i) the costimulatory domain, or the functional portion or variant thereof,
(ii) an effector domain from CD3ζ, or a functional portion or variant thereof, and
(iii) the SH2 domain or functional portion or variant thereof.

19. The fusion protein of claim 18, wherein the effector domain or functional portion or variant thereof is disposed between the costimulatory domain or functional portion or variant thereof and the SH2 domain or functional portion or variant thereof.

20. The fusion protein of claim 18, wherein (a) the SH2 domain or functional portion or variant thereof is disposed between the costimulatory domain or functional portion or variant thereof and the effector domain or functional portion or variant thereof, or (b) the costimulatory domain or functional portion or variant thereof is disposed between the SH2 domain or the functional portion or variant thereof and the effector domain or functional portion or variant thereof.

21. The fusion protein of any one of claims 18-20, further comprising a linker disposed between (i) and (ii), between (ii) and (iii), and/or between (i) and (iii).

22. The fusion protein of any one of claims 18-21, wherein the SH2 domain or functional portion or variant thereof is from Grb2, and optionally comprises an amino acid sequence having at least 75% identity to, comprising, or consisting of the amino acid sequence set forth in SEQ ID NO.:8 or 9.

23. The fusion protein of any one of claims 17-19, 21, or 22, wherein the intracellular component comprises, in an amino-terminal to carboxy-terminal direction, (i)-(iv):
(i) a costimulatory domain from 4-1BB, or a functional portion or variant thereof;
(ii) an effector domain from CD3ζ, or a functional portion or variant thereof;
(iii) an optional linker; and
(iv) an SH2 domain from Grb2, or a functional portion or variant thereof.

24. The fusion protein of claim 23, wherein the intracellular domain further comprises a junction amino acid, wherein the junction amino acid is optionally disposed between (i) and (ii), between (ii) and (iii), between (iii) and (iv), or any combination thereof.

25. The fusion protein of any one of claims 1-24, wherein the transmembrane domain or functional portion or variant thereof comprises or is:
(i) a CD28 transmembrane domain, or a functional portion or variant thereof;
(ii) a CD27 transmembrane domain, or a functional portion or variant thereof;
(iii) a CD4 transmembrane domain, or a functional portion or variant thereof; or
(iv) a CD8 transmembrane domain, or a functional portion or variant thereof, or any combination thereof.

26. The fusion protein of any one of claims 1-25, wherein the extracellular component further comprises:
(i) a CH1 domain, or a functional variant or portion thereof;
(ii) a CH2 domain, or a functional variant or portion thereof;
(iii) a CH3 domain, or a functional variant or portion thereof;
(iv) a CL domain, or a functional variant or portion thereof;
(v) a CD8 extracellular domain, or a functional variant or portion thereof;
(vi) a CD28 extracellular domain, or a functional variant or portion thereof;
(vii) a CD4 extracellular domain, or a functional variant or portion thereof
(viii) an IgG hinge, or a functional variant or portion thereof;
(ix) a type II C-lectin interdomain (stalk) region, or a functional variant or portion thereof;
(x) a cluster of differentiation (CD) molecule stalk region or a functional variant thereof;
(xi) a linker, optionally a glycine-serine linker comprising from about one to about ten repeats of GlyxSery, wherein X and Y are each independently from one to ten; or
(xii) any combination of (i)-(xi).

27. The fusion protein of any one of claims 1-26, wherein the extracellular domain comprises a linker disposed between the binding domain and the transmembrane domain.

28. The fusion protein of claim 27, wherein the linker comprises a hinge region or a portion thereof.

29. The fusion protein of any one of claims 1-28, wherein the binding domain comprises or consists of a scFv, a Fab, a scFab, a scTCR, a scTv, a DARPin, a ¹⁰FNIII domain, a VHH, a VNAR, a receptor ectodomain, or a ligand.

30. The fusion protein of claim 29, wherein the binding domain comprises a scFv.

31. The fusion protein of any one of claims 1-30, wherein the binding domain is chimeric, human, or humanized.

32. The fusion protein of any one of claims 1-31, wherein target bound by the binding domain comprises: (i) an antigen that is expressed by or is otherwise associated with a cancer; (ii) an autoimmune antigen; or (iii) an antigen that is associated with an infection, such as a viral, bacterial, fungal, or parasitic antigen.

33. The fusion protein of claim 32, wherein the cancer comprises a solid tumor.

34. The fusion protein of any one of claims 1-33, wherein the antigen is selected from a ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, mesothelin, NY-ESO, 5T4, BCMA, FAP, Carbonic anhydrase 9, BRAF, α-fetoprotein, MAGE-A3, MAGE-A4, SSX-2, PRAME, HA-1, β2M, ETA, tyrosinase, KRAS, NRAS, or CEA antigen.

35. The fusion protein of claim 34, wherein the antigen is a ROR1 antigen.

36. The fusion protein of claim 34, wherein the antigen is a CD19 antigen.

37. The fusion protein of any one of claims 1-30, further comprising a protein tag, wherein the protein tag is optionally disposed in the extracellular component of the fusion protein, further optionally between the binding domain and the transmembrane domain, still further optionally between the binding domain and a linker.

38. An isolated polynucleotide encoding the fusion protein of any one of claims 1-37.

39. The isolated polynucleotide of claim 38, further comprising a polynucleotide encoding a transduction marker.

40. The isolated polynucleotide of claim 39, wherein the encoded transduction marker comprises EGFRt, CD19t, CD34t, or NGFRt.

41. The isolated polynucleotide of any one of claims 39 or 40, further comprising a polynucleotide encoding a self-cleaving polypeptide.

42. The isolated polynucleotide of claim 41, wherein the fusion protein-encoding polynucleotide is separated from the transduction marker-encoding polynucleotide by the polynucleotide encoding a self-cleaving polypeptide.

43. The isolated polynucleotide of claim 41 or 42, wherein the encoded self-cleaving polypeptide comprises a P2A, an F2A, a T2A, an E2A, or a variant thereof.

44. The isolated polynucleotide of any one of claims 38-43, wherein the polynucleotide is codon optimized for expression in a host cell.

45. The isolated polynucleotide of any one of claims 38-44, wherein the polynucleotide comprises a nucleotide sequence having at least about 75% identity to the nucleotide sequence set forth in any one of SEQ ID NOs:132-146 or 149.

46. An expression vector, comprising the isolated polynucleotide of any one of claims 38-45 operably linked to an expression control sequence.

47. The vector of claim 46, wherein the vector is capable of delivering the polynucleotide to a host cell.

48. The expression vector of claim 47, wherein the host cell is a hematopoietic progenitor cell or a human immune system cell.

49. The expression vector of claim 48, wherein the human immune system cell comprises a CD4+ T cell, a CD8+ T cell, a CD4− CD8− double negative T cell, a γδ T cell, a natural killer cell, a natural killer T cell, a dendritic cell, or any combination thereof.

50. The expression vector of claim 49, wherein the T cell comprises a naïve T cell, a central memory T cell, a stem cell memory T cell, an effector memory T cell, or any combination thereof.

51. The expression vector of any one of claims 46-50, wherein the vector is a viral vector.

52. The expression vector of claim 51, wherein the viral vector is a lentiviral vector or a γ-retroviral vector.

53. A host cell, comprising the polynucleotide of any one of claims 38-45 and/or transduced with the expression vector of any one of claims 46-52.

54. A host cell, expressing at its cell surface the fusion protein of any one of claims 1-37.

55. The host cell of claim 54, further expressing a transduction marker at its cell surface.

56. The host cell of any one of claims 53-55, wherein the host cell comprises a hematopoietic progenitor cell or a human immune system cell.

57. The host cell of claim 56, wherein the human immune system cell comprises a CD4+ T cell, a CD8+ T cell, a CD4− CD8− double negative T cell, a γδ T cell, a natural killer cell, a natural killer T cell, a dendritic cell, or any combination thereof.

58. The host cell of claim 56 or 57, wherein the host cell comprises a T cell.

59. The host cell of claim 57 or 58, wherein the T cell comprises a naïve T cell, a central memory T cell, a stem cell memory T cell, an effector memory T cell, or any combination thereof.

60. The host cell of any one of claims 53-59, comprising a chromosomal gene knockout or a mutation of: a PD-1 gene; a LAG3 gene; a TIM3 gene; a CTLA4 gene; an HLA component gene; a TCR component gene, or any combination thereof.

61. A composition, comprising the fusion protein of any one of claims 1-37 and a pharmaceutically acceptable carrier, excipient, or diluent.

62. A composition, comprising the host cell of any one of claims 53-60 and a pharmaceutically acceptable carrier, excipient, or diluent.

63. A unit dose, comprising an effective amount of the host cell of any one of claims 53-60, or of the composition of claim 61 or 62.

64. The composition of claim 62 or the unit dose of claim 63, comprising (i) a composition comprising at least about 30% CD4+T host cells, combined with (ii) a composition comprising at least about 30% engineered CD8+T host cells, in about a 1:1 ratio.

65. A method of treating a disease or condition in a subject, the method comprising administering to the subject the host cell of any one of claims 53-60, the composition of claim 61, 62, or 64, or the unit dose of claim 63 or 64, wherein the disease or condition is characterized by the presence of the target that is bound by the binding domain of the fusion protein.

66. A method of eliciting an immune response against the target that is specifically bound by the fusion protein of any one of claims 1-37, the method comprising administering to a subject comprising or expressing the target, the host cell of any one of claims 53-60, the composition of claim 61, 62, or 64, or the unit dose of claim 63 or 64.

67. The method of claim 65, wherein the disease or condition comprises or is a hyperproliferative disease or a proliferative disease.

68. The method of claim 65 or 67, wherein the disease or condition is a cancer.

69. The method of claim 68, wherein the cancer comprises a carcinoma, a sarcoma, a glioma, a lymphoma, a leukemia, a myeloma, or any combination thereof.

70. The method of claim 68 or 69, wherein the cancer comprises a cancer of the head or neck, melanoma, pancreatic cancer, cholangiocarcinoma, hepatocellular cancer, breast cancer including triple-negative breast cancer (TNBC), gastric cancer, non-small-cell lung cancer, prostate cancer, esophageal cancer, mesothelioma, small-cell lung cancer, colorectal cancer, glioblastoma, or any combination thereof.

71. The method of any one of claims 68-70, wherein the cancer comprises Askin's tumor, sarcoma botryoides, chondrosarcoma, Ewing's sarcoma, PNET, malignant hemangioendothelioma, malignant schwannoma, osteosarcoma, alveolar soft part sarcoma, angiosarcoma, cystosarcoma phyllodes, dermatofibrosarcoma protuberans (DFSP), desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, undifferentiated pleomorphic sarcoma, malignant peripheral nerve sheath tumor (MPNST), neurofibrosarcoma, rhabdomyosarcoma, synovial sarcoma, undifferentiated pleomorphic sarcoma, squamous cell carcinoma, basal cell carcinoma, adenocarcinoma, linitis plastic, vipoma, cholangiocarcinoma, hepatocellular carcinoma, adenoid cystic carcinoma, renal cell carcinoma, Grawitz tumor, ependymoma, astrocytoma, oligodendroglioma, brainstem glioma, optice nerve glioma, a mixed glioma, Hodgkin's lymphoma, a B-cell lymphoma, non-Hodgkin's lymphoma (NHL), Burkitt's lymphoma, small lymphocytic lymphoma (SLL), diffuse large B-cell lymphoma, follicular lymphoma, immunoblastic large cell lymphoma, precursor B-lymphoblastic lymphoma, and mantle cell lymphoma, Waldenström's macroglobulinemia, CD37⁺ dendritic cell lymphoma, lymphoplasmacytic lymphoma, splenic marginal zone lymphoma, extra-nodal marginal zone B-cell lymphoma of mucosa-associated (MALT) lymphoid tissue, nodal marginal zone B-cell lymphoma, mediastinal (thymic) large B-cell lymphoma, intravascular large B-cell lymphoma, primary effusion lymphoma, adult T-cell lymphoma, extranodal NK/T-cell lymphoma, nasal type, enteropathy-associated T-cell lymphoma, hepatosplenic T-cell lymphoma, blastic NK cell lymphoma, Sezary syndrome, angioimmunoblastic T cell lymphoma, anaplastic large cell lymphoma, or any combination thereof.

72. The method of any one of claims 68-71, wherein the cancer comprises a solid tumor.

73. The method of claim 72, wherein the solid tumor is a sarcoma or a carcinoma.

74. The method of claim 73, wherein the solid tumor is selected from: chondrosarcoma; fibrosarcoma (fibroblastic sarcoma); Dermatofibrosarcoma protuberans (DFSP); osteosarcoma; rhabdomyosarcoma; Ewing's sarcoma; a gastrointestinal stromal tumor; Leiomyosarcoma; angiosarcoma (vascular sarcoma); Kaposi's sarcoma; liposarcoma; pleomorphic sarcoma; or synovial sarcoma.

75. The method of claim 73 or 74, wherein the solid tumor is selected from a lung carcinoma (e.g., Adenocarcinoma, Squamous Cell Carcinoma (Epidermoid Carcinoma); Squamous cell carcinoma; Adenocarcinoma; Adenosquamous carcinoma; anaplastic carcinoma; Large cell carcinoma; Small cell carcinoma; a breast carcinoma (e.g., Ductal Carcinoma in situ (non-invasive), Lobular carcinoma in situ (non-invasive), Invasive Ductal Carcinoma, Invasive lobular carcinoma, Non-invasive Carcinoma); a liver carcinoma (e.g., Hepatocellular Carcinoma, Cholangiocarcinomas or Bile Duct Cancer); Large-cell undifferentiated carcinoma, Bronchioalveolar carcinoma); an ovarian carcinoma (e.g., Surface epithelial-stromal tumor (Adenocarcinoma) or ovarian epithelial carcinoma (which includes serous tumor, endometrioid tumor and mucinous cystadenocarcinoma), Epidermoid (Squamous cell carcinoma), Embryonal carcinoma and choriocarcinoma (germ cell tumors)); a kidney carcinoma (e.g., Renal adenocarcinoma, hypernephroma, Transitional cell carcinoma (renal pelvis), Squamous cell carcinoma, Bellini duct carcinoma, Clear cell adenocarcinoma, Transitional cell carcinoma, Carcinoid tumor of the renal pelvis); an adrenal carcinoma (e.g., Adrenocortical carcinoma), a carcinoma of the testis (e.g., Germ cell carcinoma (Seminoma, Choriocarcinoma, Embryonal carciroma, Teratocarcinoma), Serous carcinoma); Gastric carcinoma (e.g., Adenocarcinoma); an intestinal carcinoma (e.g., Adenocarcinoma of the duodenum); a colorectal carcinoma; or a skin carcinoma (e.g., Basal cell carcinoma, Squamous cell carcinoma).

76. The method of claim 73 or 75, wherein the solid tumor is an ovarian carcinoma, an ovarian epithelial carcinoma, a cervical adenocarcinoma or small cell carcinoma, a pancreatic carcinoma, a colorectal carcinoma (e.g., an adenocarcinoma or squamous cell carcinoma), a lung carcinoma, a breast ductal carcinoma, or an adenocarcinoma of the prostate.

77. The method of any one of claims 65-76, wherein the host cell is an allogeneic cell, a syngeneic cell, or an autologous cell.

78. The method of any one of claims 65-77, wherein the method comprises administering a plurality of unit doses to the subject.

79. The method of claim 78, wherein the plurality of unit doses are administered at intervals between administrations of about two, three, four, five, six, seven, eight, or more weeks.

80. The method according to any one of claims 65-79, wherein the unit dose comprises about 10⁵ cells/m² to about 10¹¹ cells/m².

81. The method of any one of claims 65-80, wherein the subject is receiving, has received, or will receive one or more of:
(i) chemotherapy;
(ii) radiation therapy;
(iii) an inhibitor of an immune suppression component;
(iv) an agonist of a stimulatory immune checkpoint agent;
(v) RNAi;
(vi) a cytokine;
(vii) a surgery;
(viii) a monoclonal antibody and/or an antibody-drug conjugate; or
(ix) any combination of (i)-(viii), in any order.

82. The fusion protein of any one of claims 1-37, the polynucleotide of any one of claims 38-45, the vector of any one of claims 46-52, the host cell of any one of claims 53-60, the composition of claim 61, 62, or 64, or the unit dose of claim 63 or 64, for use in the treatment of a disease or disorder in a subject, wherein the disease or condition is characterized by the presence of the target that is bound by the binding domain of the fusion protein.

83. The fusion protein of any one of claims 1-37, the polynucleotide of any one of claims 38-45, the vector of any one of claims 46-52, the host cell of any one of claims 53-60, the composition of claim 61, 62, or 64, or the unit dose of claim 63 or 64, for use in the manufacture of a medicament for the treatment of a disease or disorder in a subject, wherein the disease or condition is characterized by or otherwise associated with the presence of the target that is bound by the binding domain of the fusion protein.
